(19) United States
(12) Patent Application Publication
(10) Pub. No.: US 2021/0363509 A1
(43) Pub. Date: Nov. 25, 2021

(54) Genome Editing by Directed Non-Homologous DNA Insertion Using a Retroviral Integrase-Cas9 Fusion Protein

(71) Applicant: UNIVERSITY OF ROCHESTER, Rochester, NY (US)

(72) Inventor: Douglas Matthew Anderson, Pittsford, NY (US)

(21) Appl. No.: 17/287,184

(22) PCT Filed: Oct. 22, 2019
(86) PCT No.: PCT/US2019/057498

§ 371 Date: Apr. 21, 2021

Related U.S. Application Data
(60) Provisional application No. 62/748,703, filed on Oct. 22, 2018.

(51) Int. Cl. C12N 9/22; C12N 9/12; C12N 15/62; C12N 15/11

(52) U.S. Cl. CPC C12N 9/22; C12N 9/12; C12N 2310/20; C12N 15/111; C07K 2319/09; C12N 15/62

(57) ABSTRACT

The present invention provides proteins, nucleic acids, systems and methods for editing genomic material.

### CROSS-REFERENCE TO RELATED APPLICATIONS

The present application claims priority to U.S. Provisional Application Ser. No. 62/748,703, filed on Oct. 22, 2018, which is incorporated by reference herein in its entirety.

### BACKGROUND OF THE INVENTION

CRISPR-Cas9 has significantly advanced our ability to rapidly alter mammalian genomes for basic research and clinical applications. CRISPR-Cas9 uses a guide-RNA to direct Cas9 to specific DNA target sequences, where it induces double-strand DNA cleavage and triggers cellular repair pathways to introduce frame-shift mutations or insert donor sequences through Homology Directed Repair (HDR). Despite these significant advances, the targeted delivery of large DNA sequences for genome editing using CRISPR-Cas9 mediated HDR remains inefficient, requires donor templates containing significant regions of flanking homology and induces the p53 DNA damage pathway (Byrne et al., 2015, NAR 43:e21; Happaniemi et al., 2018, Nat Med 24:927-30; Ihry et al., 2018, Nat Med 24:939-46). Together, these significantly limit the efficiency of CRISPR-Cas9 genome editing. Accordingly, there exists a need for improved integrated genome editing.

In contrast, the lentiviral enzyme Integrase (IN) is both necessary and sufficient to catalyze the insertion of large lentiviral genomes into host cellular DNA, through a process which does not require target sequence homology. IN-mediated insertion of lentiviral DNA occurs with little DNA target sequence specificity, due in part to its C-terminal domain which binds non-specifically to DNA (Lutzke & Plasterk 1998, J Virol 72:4841-48).

Current limitations with gene therapy technologies have prevented the treatment of most human monogenetic diseases. CRISPR-Cas9 gene editing has been a recent focus for the development of therapeutic approaches to correct deleterious mutations mammalian genomes. This remains a significant challenge due to the numerous patient-specific mutations within the human genome that can give rise to diseases and disorders. CRISPR guide-RNAs designed to target exon-intron boundaries can allow for exon-skipping strategies to target groups of these mutations, however, the efficacy of these strategies remain to be tested and are not applicable to all patients.

Transgenic expression of many genes can both prevent and reverse disease outcomes in animal models, however the large size of some genes greatly exceeds the size limit of traditional gene editing approaches, such as CRISPR-Cas9 or traditional viral gene therapy approaches, such as AAV (˜4.9 kb limit), preventing its use for human gene therapy. Approaches using smaller engineered genes delivered by AAV are currently in clinical trials, however it remains to be determined if these strategies offer long term restoration and are only applicable to patients with specific mutations.

In contrast, lentiviral vectors are capable of delivering large gene and allow for permanent correction by integrating into host genomes. However, the current random nature of lentiviral integration has the potential to cause off-target mutations and disease, which has prevented their use for clinical applications (Milone et al., 2018, Leukemia 23:1529-41). Lentiviral sequences are inserted into host genomes by the virus-encoded enzyme Integrase (IN), which utilizes a non-specific DNA binding domain required for genome integration (Andrake et al., 2015, Annu Rev Virol 2:241-64).

Accordingly, there exists a need for improved editing genomic material. The present invention meets this need.

### SUMMARY OF THE INVENTION

In one aspect, the invention provides a fusion protein. In one embodiment, the fusion protein comprises a retroviral integrase (IN), or a fragment thereof having a first amino acid sequence; a CRISPR-associated (Cas) protein having a second amino acid sequence; and a nuclear localization signal (NLS) having a third amino acid sequence.

In one embodiment, the retroviral IN is selected from the group consisting of human immunodeficiency virus (HIV) IN, Rous sarcoma virus (RSV) IN, Mouse mammary tumor virus (MMTV) IN, Moloney murine leukemia virus (MoLV) IN, bovine leukemia virus (BLV) IN, Human T-lymphotropic virus (HTLV) IN, avian sarcoma leukosis virus (ASLV) IN, feline leukemia virus (FLV) IN, xenotropic murine leukemia virus-related virus (XMLV) IN, simian immunodeficiency virus (SIV) IN, feline immunodeficiency virus (FIV) IN, equine infectious anemia virus (EIAV) IN, Prototype foamy virus (PFV) IN, simian foamy virus (SFV) IN, human foamy virus (HFV) IN, walleye dermal sarcoma virus (WDSV) IN, and bovine immunodeficiency virus (BIV) IN.

In one embodiment, the retroviral IN fragment comprises the IN N-terminal domain (NTD), and the IN catalytic core domain (CCD). In one embodiment, the retroviral IN comprises a sequence at least 70% identical to one of SEQ ID NOs:1-40. In one embodiment, the retroviral IN comprises a sequence of one of SEQ ID NOs:1-40.

In one embodiment, the Cas protein is selected from the group consisting of Cas9, Cas13, and Cpf1. In one embodiment, the Cas protein is catalytically deficient (dCas). In one embodiment, the Cas protein comprises a sequence at least 95% identical to one of SEQ ID NOs:41-46. In one embodiment, the Cas protein comprises a sequence of one of SEQ ID NOs:41-46.

In one embodiment, the NLS is a retrotransposon NLS. In one embodiment, the retrotransposon NLS is Ty1 or Ty2 NLS. In one embodiment, the NLS is a Ty1-like NLS. In one embodiment, the NLS comprises a sequence at least 70% identical to one of SEQ ID NOs:47-56, 254-257, and 275-887. In one embodiment, the NLS comprises a sequence of one of SEQ ID NOs:47-56, 254-257, and 275-887.

In one embodiment, the fusion protein comprises a sequence at least 70% identical to one of SEQ ID NOs:57-98. In one embodiment, the fusion protein comprises a sequence of one of SEQ ID NOs:57-98.

In one aspect, the invention provides a nucleic acid encoding a fusion protein of the invention. In one embodiment, the nucleic acid comprises a sequence at least 70% identical to one of SEQ ID NOs:155-196. In one embodiment, the nucleic acid comprises a sequence selected from SEQ ID NOs:155-196.

In one aspect, the invention provides a method of editing genetic material. In one embodiment, the method comprises administering to the genetic material: (a) a fusion protein of the invention or a nucleic acid molecule encoding a fusion protein of the invention, (b) a guide nucleic acid comprising a targeting nucleotide sequence complimentary to a target region in the genetic material, and (c) a donor template nucleic acid comprising a U3 sequence, a U5 sequence and a donor template sequence. In one embodiment, the method of editing genetic material is an in vitro method. In one embodiment, the method of editing genetic material is an in vivo method.

In one aspect, the invention provides a system for editing genetic material. In one embodiment, the system comprises, in one or more vectors, (a) a nucleic acid sequence encoding a fusion protein of the invention, (b) a nucleic acid sequence coding a CRISPR-Cas system guide RNA, and (c) a nucleic acid sequence coding a donor template nucleic acid, wherein the donor template nucleic acid comprises a U3 sequence, a U5 sequence and a donor template sequence. In one embodiment, the fusion protein comprises a retroviral integrase (IN), or a fragment thereof; a CRISPR-associated (Cas) protein, and a nuclear localization signal (NLS). In one embodiment, the nucleic acids are on the same vector. In one embodiment, the nucleic acids are on different vectors.

In one embodiment, the CRISPR-Cas system guide RNA substantially hybridizes to a target DNA sequence in the gene. In one embodiment, the U3 sequence and U5 sequence are specific to the retroviral IN.

In aspect, the invention provides a system for delivering genome editing components. In one embodiment, the system comprises: (a) a packaging plasmid comprising sequence encoding a gag-pol polyprotein comprising integrase fused to a catalytically dead Cas (dCas) protein; (b) transfer plasmid comprising a sequence encoding a donor sequence, a 5′LTR and a 3′LTR; and (c) an envelope plasmid comprising a nucleic acid sequence encoding an envelope protein. In one embodiment, the packaging plasmid further comprises a sequence encoding a guide RNA sequence.

In one embodiment, the system comprises (a) a packaging plasmid comprising sequence encoding a gag-pol polyprotein; (b) transfer plasmid comprising a sequence encoding a donor sequence, a 5′LTR and a 3′LTR; (c) an envelope plasmid comprising a nucleic acid sequence encoding an envelope protein; and (d) a VPR-IN-dCas plasmid comprises a nucleic acid sequence encoding a fusion protein comprising VPR, integrase, and catalytically dead Cas (dCas). In one embodiment, the VPR-IN-dCas plasmid further comprises a sequence encoding a guide RNA sequence.

In one embodiment, the system comprises (a) a packaging plasmid comprising nucleic acid sequence encoding a gag-pol polyprotein; (b) transfer plasmid comprising a nucleic acid sequence encoding an guide RNA, a fusion protein comprising integrase and a catalytically dead Cas, a 5′LTR and a 3′LTR; and (c)a n envelope plasmid comprising a nucleic acid sequence encoding an envelope protein.

### BRIEF DESCRIPTION OF THE DRAWINGS

The following detailed description of embodiments of the invention will be better understood when read in conjunction with the appended drawings. It should be understood, however, that the invention is not limited to the precise arrangements and instrumentalities of the embodiments shown in the drawings.

FIG. 1, comprising FIG. 1A through FIG. 1C, depicts experimental results demonstrating enhanced nuclear localization of retroviral Integrase-dCas9 fusion proteins for editing of mammalian genomic DNA. FIG. 1A depicts a schematic of the IN-dCas9 fusion proteins. FIG. 1B depicts the nuclear localization of IN-dCas9 fusion proteins. FIG. 1C depicts experimental results demonstrating the enzymatic activity of INAC-dCas9 fusion protein to integrate an IRES-mCherry template targeted to the 3′UTRE of EF1-alpha in HEK293 cells.

FIG. 2, depicts a schematic of the nucleic acid editing technology showing that the fusion of viral Integrase(IN) with CRISPR-dCas9 allows for the integration of large DNA sequences in a target specific manner. This approach allows for the safe and permanent delivery of large gene sequences that normally exceed the limit of non-integrating AAV vectors.

FIG. 3 depicts the experimental design and experimental results of the GFP reporter cell line used quantify and characterize the fidelity of individual integration events in mammalian cells.

FIG. 4 depicts a schematic of the CRISPER-Cas9-mediated homology directed repair and the retroviral integrase-mediated random DNA integration.

FIG. 5 depicts a schematic of the Integrase-Cas genome editing.

FIG. 6 depicts schematics of the donor vector, generating blunt-ended templates, and generating 3′-processed templates.

FIG. 7 depicts the experimental design of the co-transfection of the INsrt templates, the IN-dCas9 vectors targeting the amilCP sequence were co-transfected into Cos7 cells.

FIG. 8 depicts the experimental design of the paired guide-RNAs specific the 3′UTR of the human EF1-alpha locus to knock-in the IGR-mCherry-2A-puromycin-pA cassette into the human HEK293 cell line and images of mCherry-positive cells 48 hours after transfection.

FIG. 9 depicts a schematic demonstrating directional editing

FIG. 10 depicts a schematic demonstrating multiplex genome editing for the generation of floxed alleles.

FIG. 11, comprising FIG. 11A through FIG. 11C, depicts experimental results demonstrating the efficiency of Ty1 NLS-like Sequences on Nuclear Localization of INΔC-Cas9 fusion proteins. FIG. 11A depicts the detection of INΔC-dCas9 fusion proteins containing a C-terminal classic SV40, Ty1 or Ty2 NLSs expressed in Cos-7 cells using an anti-FLAG antibody. FIG. 11B depicts Ty1 NLS-like sequences isolated from yeast proteins can provide robust nuclear localization (MAK11) or no apparent localizing activity (INO4 and STH1). FIG. 11C depicts sequences of Ty1, Ty2 and Ty1 NLS-like sequences. Ty1 and Ty2 are highly conserved in both length and residue composition. Scale bars=10 μm.

FIG. 12, comprising FIG. 12A through FIG. 12C, depicts experimental results demonstrating that the Ty1 NLS enhances Cas9 DNA editing in mammalian cells. FIG. 12A depicts a diagram of the px330 CRISPR-Cas9 expression plasmid which encodes an hU6-driven single guide-RNA (sgRNA) and CAG driven Cas9 protein containing an N-terminal 3×FLAG tag, SV40 NLS and C-terminal NPM NLS. The Ty1 NLS was cloned in place of the NPM NLS in px330 (px330-Ty1). FIG. 12B depicts a frame-shift activated luciferase reporter was generated in which an upstream 20 nt target sequence (ts) interrupts the open reading from of a downstream luciferase open reading frame. Frameshifts induced by non-homologous end joining (NHEJ) reframe the downstream reporter and allow for Luciferase expression. FIG. 12C depicts co-expression of the frameshift-responsive luciferase reporter and px330 containing a single guide-RNA specific to the target sequence resulted in a ˜20-fold activation of luciferase activity, relative to a non-targeting sgRNA. Co-expression of px330-Ty1 resulted in a ˜44% enhancement over px330.

FIG. 13, comprising FIG. 13A through FIG. 13E, depicts genome targeting strategies for editing. Integration of DNA donor sequences can be targeted to different genome locations dependent upon the desired application. FIG. 13A depicts delivery of a DNA donor sequence carrying a gene cassette could be targeted to an intergenic ‘safe harbor’ locus to prevent disruption of neighbor or essential gene expression. FIG. 13B depicts delivery of a DNA donor sequence carrying a gene cassette could be targeted to a non-essential ‘safe harbor’ locus to prevent disruption of neighbor or essential gene expression. FIG. 13C depicts integration of a DNA sequence encoding a splice acceptor sequence (SA) could be delivered to an intron region of a gene (for example, the disease gene locus), which would allow for expression of the integrated sequence and prevent expression of the downstream sequence. FIG. 13D depicts integration of a DNA sequence encoding a splice acceptor sequence (SA) could be delivered to an intron region of a gene (for example, the disease gene locus), which would allow for expression of the integrated sequence and prevent expression of the downstream sequence. FIG. 13E depicts integration of a DNA donor sequence containing and Internal Ribosome Entry Sequence (IRES) into the 3′ UTR could allow for expression without disrupting expression from the endogenous locus.

FIG. 14 depicts a diagram of the lentiviral lifecycle. Lentivirus, a subclass of retrovirus, are single-stranded RNA viruses which integrate a permanent double-stranded DNA(dsDNA) copy of their proviral genomes into host cellular DNA. Following viral transduction, lentiviral RNA genomes are copied as blunt-ended dsDNA by viral-encoded reverse transcriptase (RT) and inserted into host genomes by Integrase I(IN). Lentiviral genomes are flanked by short (˜20 base pair) sequence motifs at their U3 and U5 termini which are required for proviral genome integration by IN. IN-mediated insertion of retroviral DNA occurs with little DNA target sequence specificity and can integrate into active gene loci, which can disrupt normal gene function and has the potential to cause disease in humans.

FIG. 15, comprising FIG. 15A through FIG. 15E, depicts genome editing in mammalian cells. Fusion of lentiviral Integrase to dCas9 allows for targeted non-homologous insertion of donor DNA sequences containing short viral termini. FIG. 15A depicts a diagram of a mammalian expression vector encoding a human U6-driven single-guide RNA (sgRNA) and Integrase-dCas9 fusion protein. FIG. 15B depicts a diagram showing a dsDNA Donor template containing an IGR IRES-mCherry-2A-Puromycin (puro) cassette flanked by U3/U5 viral motifs. FIG. 15C depicts a schematic Integrase-Cas9-mediated integration of this donor template into a CMV-eGFP reporter transgene stably expressed in COS-7 cells. FIG. 15D depicts a schematic demonstrating integrase-Cas9-mediated integration of this donor template into a CMV-eGFP reporter transgene stably expressed in COS-7 can result in disruption of eGFP expression while allowing mCherry expression. FIG. 15E depicts experimental results demonstrating loss of eGFP expression and gain of mCherry expression in edited COS-7 cells.

FIG. 16, comprising FIG. 16A through FIG. 16C, depicts traditional lentiviral gene delivery systems. FIG. 16A depicts a diagram of a lentiviral genome, which encodes viral proteins between flanking long terminal repeats (LTRs). FIG. 16B and FIG. 16C depicts schematics demonstrating that lentiviral genomes have been harnessed as a robust gene delivery tool. Lentiviral particles can be used to package, deliver and stably express donor transgene sequences. For lentiviral vector gene expression systems, viral polyproteins are removed from the viral genome and expressed using separate mammalian expression plasmids. Donor DNA sequences of interest can then be cloned in place of viral polyproteins between the flanking LTR sequences. Co-transfection of these vectors in mammalian packaging cells allows for the formation of lentiviral particles capable of delivering and integrating the encoded donor sequence, however do not require the coding information for Integrase and other viral proteins necessary for subsequent viral propagation. Lentiviral particles are a natural vector for the delivery of both viral proteins (ex. integrase and reverse transcriptase) and dsDNA donor sequences, which contain the necessary viral end sequences required for integrase-mediated insertion into mammalian cells. FIG. 16B depicts the generation of lentiviral vectors. FIG. 16C depicts the transduction of the lentiviral particle which deliver and stably express donor transgene sequences.

FIG. 17, comprising FIG. 17A through FIG. 17C, depicts targeted lentiviral integration. Existing lentiviral delivery systems can be modified to incorporate editing components for the purpose of targeted lentiviral donor template integration for genome editing in mammalian cells. FIG. 17A depicts one approach in which dCas9 is directly fused to Integrase (or to Integrase lacking its C-terminal non-specific DNA binding domain) within a lentiviral packaging plasmid (ex. psPax2) encoding the gag-pol polyprotein. FIG. 17B depicts that the modified gag-pol polyprotein is translated with other viral components as a polyprotein, loaded with guide-RNA and packaged into lentiviral particles. For this approach, the IN-dCas9 fusion protein retains the sequences necessary for protease cleavage (PR), and thus is cleaved normally from the gag-pol polyprotein during particle maturation. Transduction of mammalian cells results in the delivery of viral proteins, including the IN-dCas9 fusion protein, sgRNA, and lentiviral donor sequence. FIG. 17C depicts that upon lentiviral transduction, reverse transcription of the ssRNA genome by reverse transcriptase generates a dsDNA sequence containing correct viral end sequences (U3 and U5) which is Integrated into mammalian genomes by the IN-dCas9 fusion protein.

FIG. 18, comprising FIG. 18A through FIG. 18C, depicts targeted lentiviral integration via fusion to viral protein. FIG. 18A depicts expression and packaging of IN-dCas9 as N-terminal and C-terminal fusions with viral proteins (for example, viral protein R, VPR) as one approach to achieving targeted lentiviral gene integration. A viral protease cleavage sequence is included between VPR and the IN-dCas9 fusion protein, so that after maturation, the IN-dCas9 will be freed from VPR. FIG. 18B depicts that co-transfection of packaging cells with lentiviral components generates viral particles containing the VPR-IN-dCas9 protein and sgRNA. The packaging plasmid required for viral particle formation (ex. psPax2) contains a mutation within Integrase to inhibit its catalytic activity in the context of the packaging plasmid, thereby preventing non-Integrase-Cas9 mediated integration. FIG. 18C depicts that upon viral transduction, the IN-dCas9 protein is delivered as protein and mediates the integration of the lentiviral donor sequences. The benefit to delivery of the IN-dCas9 fusion and sgRNA as a riboprotein is that it is only be transiently expressed in the target cell.

FIG. 19, comprising FIG. 19A through FIG. 19C, depicts targeted lentiviral integration via incorporation into transfer plasmid. FIG. 19A depicts that expression of IN-dCas9 fusion protein and/or guide-RNA from within the viral transfer plasmid (or other viral vector, such as AAV) is one approach to achieving targeted lentiviral gene integration. FIG. 19B depicts that in this approach, the transfer plasmid containing the IN-dCas9 fusion protein and sgRNA is co-transfected with packaging and envelope plasmids required to generate lentiviral particles. If using a lentivirus, the packaging plasmid contains a catalytic mutation within Integrase to inhibit non-specific integration. FIG. 19C depicts that upon transduction of a mammalian cell, expression of the IN-dCas9 fusion protein and sgRNA generates components capable of targeting its own viral donor vector for targeted integration (self-integration). This method is used for targeted gene disruption or as a gene drive.

FIG. 20, comprising FIG. 20A through FIG. 20D, depicts co-delivery of a lentiviral donor sequence. FIG. 20A depicts co-transduction with a lentiviral particle encoding a donor DNA sequence could serve as the integrated donor template. FIG. 20B and FIG. 20C depict that prevention of self-integration of its own viral encoding sequence in this approach could be achieved by using Integrase enzymes from different retroviral family members and their corresponding transfer plasmids. FIG. 20B depicts generation of an HIV lentiviral particle encoding an IN(FIV)-dCas9 fusion protein. FIG. 20C depicts generation of an FIV lentiviral particle comprising an FIV transfer plasmid. FIG. 20D depicts that the HIV lentiviral particle encoding an IN(FIV)-dCas9 fusion protein is utilized to integrate an FIV donor template encoded within an FIV lentiviral particle.

FIG. 21 depicts targeted lentiviral integration in primary mammalian cells. This data demonstrates lentiviral packaging, delivery and targeted integration of a lentiviral donor template encoding an IRES-tdTO cassette into the ROSA26^{mG/+} locus in mouse embryonic fibroblasts. After two days, ubiquitous red fluorescent protein expression was detectable in MEFs transduced with lentivirus encoding the IRES-tdTO reporter, but retained GFP fluorescence. Remarkably, seven days post-transduction, tdTO red fluorescent cells were detectable in in culture, which lacked green fluorescence in ROSA26^{mG/+} primary cells.

FIG. 22 depicts targeted lentiviral integration in a mammalian stable cell line. This data demonstrates lentiviral packaging, delivery and targeted integration of a lentiviral donor template encoding an IRES-tdTO cassette into a stably expressed CMV-eGFP in COS-7 cells.

FIG. 23, comprising FIG. 23A through FIG. 23C depicts DNA Binding Domains for Targeted Integration of Lentiviral Particles. Replacement of the non-specific DNA binding domain of Integrase with the programmable DNA binding domain of dCas9 allows for targeted integration of dsDNA donor templates via delivery in lentiviral particles. Alternative DNA binding domains (such as TALENs) may be utilized for targeted integration as fusions to viral Integrase. Using a similar lentiviral production approach, replacement of dCas9 in our previous packaging strategies with TALENs targeting a specific sequence. FIG. 23A depicts TALENs packaged and delivered as a fusion to Integrase in the context of the gag-pol polyprotein. FIG. 23B depicts TALENs packaged and delivered as a fusion to Integrase as a fusion to a viral protein. FIG. 23C depicts TALENs packaged and delivered as a fusion to Integrase encoded within the transfer plasmid.

FIG. 24, comprising FIG. 24A through FIG. 24C, depicts experimental results demonstrating that the Ty1 NLS enhances Cas9 DNA editing in mammalian cells. FIG. 24A depicts a diagram of the px330 CRISPR-Cas9 expression plasmid which encodes an hU6-driven single guide-RNA (sgRNA) and CAG driven Cas9 protein containing an N-terminal 3×FLAG tag, SV40 NLS and C-terminal NPM NLS. The Ty1 NLS was cloned in place of the NPM NLS in px330 (px330-Ty1). FIG. 24B depicts results demonstrating a frame-shift activated luciferase reporter was generated in which an upstream 20 nt target sequence (ts) interrupts the open reading from of a downstream luciferase open reading frame. Frameshifts induced by non-homologous end joining (NHEJ) reframe the downstream reporter and allow for Luciferase expression. FIG. 24C depicts results demonstrating co-expression of the Frameshift-responsive luciferase reporter and px330 containing a single guide-RNA specific to the target sequence resulted in a ˜20 fold activation of luciferase activity, relative to a non-targeting sgRNA. Co-expression of px330-Ty1 resulted in a ˜44% enhancement over px330.

FIG. 25 depicts a schematic demonstrating TALENs can be utilized to direct retroviral integrase-mediated integration of a donor DNA template

FIG. 26 depicts a schematic of the plasmid DNA integration assay.

FIG. 27 depicts experimental data demonstrating that TALEN pair separated by 16 bp resulted in ˜6 fold more Chloramphenicol-resistant colonies, whereas a TALEN pair separated by 28 bp was similar to untargeted integrase

FIG. 29, comprising FIG. 29A through FIG. 29C, depicts experimental results. FIG. 29A depicts expression of amilCP chromoprotein in E coli results in purple E coli (white arrowhead). Integrase-Cas-mediated integration of donor sequences containing viral ends disrupt amilCP expression (orange arrowhead) (growth on kanamycin plates). FIG. 29B depicts integration of Insrt IGR-CAT donor template with either blunt ends (ScaI cleaved) or 3′ Processing mimic (FauI cleaved) ends into pCRII-amilCP reporter in mammalian cells. Interestingly, deletion of the C-terminal non-specific DNA binding domain, as a fusion to dCas9, does not inhibit Integrase-Cas mediated integration. Use of ends that mimic 3′ Processing show ˜2 fold increase in CAT resistant clones. FIG. 29C depicts an assessment of Integrase mutations on Integrase-Cas-mediated integration in plasmid DNA. Dimerization inhibiting mutations (E85G and E85F) do not disrupt Integrase-Cas-mediated integration using double guide-RNA targeted integration of IGR-CAT donor template into amilCP. However, the IN E87G mutation cannot be rescued by paired targeting sgRNAs. Interestingly, a tandem INΔC fusion to dCas9 (tdINΔC-dCas9) shows ˜2 fold enhanced integration.

### DETAILED DESCRIPTION OF THE INVENTION

The present invention relates to fusion proteins, nucleic acids encoding fusion proteins, systems and methods for editing genetic material. In one embodiment, the invention relates to retroviral integrase (IN)-CRISPR-associated (Cas) fusion proteins and nucleic acid molecules encoding retroviral IN-Cas fusion proteins. In one embodiment, the IN-Cas fusion protein further comprises a nuclear localization signal (NLS).

The fusion proteins, nucleic acid molecules, systems and methods of the invention have the ability to deliver donor DNA sequences to targeted genome locations. Further, the invention eliminates the need for homology arms and relies on targeting by guide-RNAs, greatly simplifying editing genetic material.

In one aspect the invention provides an IN-Cas fusion protein. In one embodiment, the fusion protein comprises a retroviral IN, or a fragment thereof having a first amino acid sequence; a Cas protein having a second amino acid sequence; and a NLS having a third amino acid sequence.

In one aspect the invention provides nucleic acid molecule encoding an IN-Cas fusion protein. In one embodiment the nucleic acid molecule comprises a first nucleic acid sequence encoding a retroviral IN, or a fragment thereof; a second nucleic acid sequence encoding a Cas protein; and a third nucleic acid sequence encoding a NLS.

In one embodiment, the retroviral IN can be human immunodeficiency virus (HIV) IN, Rous sarcoma virus (RSV) IN, Mouse mammary tumor virus (MMTV) IN, Moloney murine leukemia virus (MoLV) IN, bovine leukemia virus (BLV) IN, Human T-lymphotropic virus (HTLV) IN, avian sarcoma leukosis virus (ASLV) IN, feline leukemia virus (FLV) IN, xenotropic murine leukemia virus-related virus (XMLV) IN, simian immunodeficiency virus (SIV) IN, feline immunodeficiency virus (FIV) IN, equine infectious anemia virus (EIAV) IN, Prototype foamy virus (PFV) IN, simian foamy virus (SFV) IN, human foamy virus (HFV) IN, walleye dermal sarcoma virus (WDSV) IN, or bovine immunodeficiency virus (BIV) IN. In one embodiment, the Cas protein is Cas9 or Cpf1. In one embodiment, the NLS is a retrotransposon NLS, such as Ty1 NLS. In one embodiment, the retrotransposon NLS increases nuclear localization.

In one aspect, the invention provides a system for editing genetic material. In one embodiment, the system comprises, in one or more vectors, a nucleic acid sequence encoding a fusion protein, wherein the fusion protein comprises a retroviral IN, or a fragment thereof; a Cas protein, and a NLS; a nucleic acid sequence coding a CRISPR-Cas system guide RNA; and a nucleic acid sequence coding a donor template nucleic acid, wherein the donor template nucleic acid comprises a U3 sequence, a U5 sequence and a donor template sequence.

In one aspect, the invention provides a method for editing genetic material. In one embodiment, the method comprising administering a nucleic acid molecule of the invention; a guide nucleic acid comprising a targeting nucleotide sequence complimentary to a target region in the gene; and a donor template nucleic acid comprising a U3 sequence, a U5 sequence and a donor template sequence.

### Definitions

Unless defined otherwise, all technical and scientific terms used herein have the same meaning as commonly understood by one of ordinary skill in the art to which this invention belongs.

Generally, the nomenclature used herein and the laboratory procedures in cell culture, molecular genetics, organic chemistry, and nucleic acid chemistry and hybridization are those well-known and commonly employed in the art.

Standard techniques are used for nucleic acid and peptide synthesis. The techniques and procedures are generally performed according to conventional methods in the art and various general references (e.g., Sambrook and Russell, 2012, Molecular Cloning, A Laboratory Approach, Cold Spring Harbor Press, Cold Spring Harbor, N.Y., and Ausubel et al., 2012, Current Protocols in Molecular Biology, John Wiley & Sons, NY), which are provided throughout this document.

The nomenclature used herein and the laboratory procedures used in analytical chemistry and organic syntheses described below are those well-known and commonly employed in the art. Standard techniques or modifications thereof are used for chemical syntheses and chemical analyses.

The term “a,” “an,” “the” and similar terms used in the context of the present invention (especially in the context of the claims) are to be construed to cover both the singular and plural unless otherwise indicated herein or clearly contradicted by the context.

“About” as used herein when referring to a measurable value such as an amount, a temporal duration, and the like, is meant to encompass variations of ±20%, or ±10%, or ±5%, or ±1%, or ±0.1% from the specified value, as such variations are appropriate to perform the disclosed methods.

“Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a sign or symptom of the disease or disorder, the frequency with which such a sign or symptom is experienced by a patient, or both, is reduced.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The terms “patient,” “subject,” “individual,” and the like are used interchangeably herein, and refer to any animal, or cells thereof whether in vitro or in vivo, amenable to the methods described herein. In certain non-limiting embodiments, the patient, subject or individual is a human.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific.

In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

A “coding region” of a mRNA molecule also consists of the nucleotide residues of the mRNA molecule which are matched with an anti-codon region of a transfer RNA molecule during translation of the mRNA molecule or which encode a stop codon. The coding region may thus include nucleotide residues comprising codons for amino acid residues which are not present in the mature protein encoded by the mRNA molecule (e.g., amino acid residues in a protein export signal sequence).

“Complementary” as used herein to refer to a nucleic acid, refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. In one embodiment, the first region comprises a first portion and the second region comprises a second portion, whereby, when the first and second portions are arranged in an antiparallel fashion, at least about 50%, at least about 75%, at least about 90%, or at least about 95% of the nucleotide residues of the first portion are capable of base pairing with nucleotide residues in the second portion. In one embodiment, all nucleotide residues of the first portion are capable of base pairing with nucleotide residues in the second portion.

The term “DNA” as used herein is defined as deoxyribonucleic acid.

The term “expression” as used herein is defined as the transcription and/or translation of a particular nucleotide sequence driven by its promoter.

The term “expression vector” as used herein refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules, siRNA, ribozymes, and the like. Expression vectors can contain a variety of control sequences, which refer to nucleic acid sequences necessary for the transcription and possibly translation of an operatively linked coding sequence in a particular host organism. In addition to control sequences that govern transcription and translation, vectors and expression vectors may contain nucleic acid sequences that serve other functions as well.

As used herein the term “wild type” is a term of the art understood by skilled persons and means the typical form of an organism, strain, gene or characteristic as it occurs in nature as distinguished from mutant or variant forms.

The term “homology” refers to a degree of complementarity. There may be partial homology or complete homology (i.e., identity). Homology is often measured using sequence analysis software (e.g., Sequence Analysis Software Package of the Genetics Computer Group. University of Wisconsin Biotechnology Center. 1710 University Avenue. Madison, Wis. 53705). Such software matches similar sequences by assigning degrees of homology to various substitutions, deletions, insertions, and other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

“Isolated” means altered or removed from the natural state. For example, a nucleic acid or a peptide naturally present in its normal context in a living animal is not “isolated,” but the same nucleic acid or peptide partially or completely separated from the coexisting materials of its natural context is “isolated.” An isolated nucleic acid or protein can exist in substantially purified form, or can exist in a non-native environment such as, for example, a host cell.

The term “isolated” when used in relation to a nucleic acid, as in “isolated oligonucleotide” or “isolated polynucleotide” refers to a nucleic acid sequence that is identified and separated from at least one contaminant with which it is ordinarily associated in its source. Thus, an isolated nucleic acid is present in a form or setting that is different from that in which it is found in nature. In contrast, non-isolated nucleic acids (e.g., DNA and RNA) are found in the state they exist in nature. For example, a given DNA sequence (e.g., a gene) is found on the host cell chromosome in proximity to neighboring genes; RNA sequences (e.g., a specific mRNA sequence encoding a specific protein), are found in the cell as a mixture with numerous other mRNAs that encode a multitude of proteins. However, isolated nucleic acid includes, by way of example, such nucleic acid in cells ordinarily expressing that nucleic acid where the nucleic acid is in a chromosomal location different from that of natural cells, or is otherwise flanked by a different nucleic acid sequence than that found in nature. The isolated nucleic acid or oligonucleotide may be present in single-stranded or double-stranded form. When an isolated nucleic acid or oligonucleotide is to be utilized to express a protein, the oligonucleotide contains at a minimum, the sense or coding strand (i.e., the oligonucleotide may be single-stranded), but may contain both the sense and anti-sense strands (i.e., the oligonucleotide may be double-stranded).

The term “isolated” when used in relation to a polypeptide, as in “isolated protein” or “isolated polypeptide” refers to a polypeptide that is identified and separated from at least one contaminant with which it is ordinarily associated in its source. Thus, an isolated polypeptide is present in a form or setting that is different from that in which it is found in nature. In contrast, non-isolated polypeptides (e.g., proteins and enzymes) are found in the state they exist in nature.

By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil). The term “nucleic acid” typically refers to large polynucleotides.

Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

By “expression cassette” is meant a nucleic acid molecule comprising a coding sequence operably linked to promoter/regulatory sequences necessary for transcription and, optionally, translation of the coding sequence.

The term “operably linked” as used herein refer to the linkage of nucleic acid sequences in such a manner that a nucleic acid molecule capable of directing the transcription of a given gene and/or the synthesis of a desired protein molecule is produced. The term also refers to the linkage of sequences encoding amino acids in such a manner that a functional (e.g., enzymatically active, capable of binding to a binding partner, capable of inhibiting, etc.) protein or polypeptide is produced.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a n inducible manner.

As used herein, “stringent conditions” for hybridization refer to conditions under which a nucleic acid having complementarity to a target sequence predominantly hybridizes with the target sequence, and substantially does not hybridize to non-target sequences. Stringent conditions are generally sequence-dependent, and vary depending on a number of factors. In general, the longer the sequence, the higher the temperature at which the sequence specifically hybridizes to its target sequence. Non-limiting examples of stringent conditions are described in detail in Tijssen (1993), Laboratory Techniques In Biochemistry And Molecular Biology-Hybridization With Nucleic Acid Probes Part 1, Second Chapter “Overview of principles of hybridization and the strategy of nucleic acid probe assay”, Elsevier, N.Y.

“Hybridization” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. The complex may comprise two strands forming a duplex structure, three or more strands forming a multi stranded complex, a single self-hybridizing strand, or any combination of these. A hybridization reaction may constitute a step in a more extensive process, such as the initiation of PCR, or the cleavage of a polynucleotide by an enzyme. A sequence capable of hybridizing with a given sequence is referred to as the “complement” of the given sequence.

An “inducible” promoter is a nucleotide sequence which, when operably linked with a polynucleotide which encodes or specifies a gene product, causes the gene product to be produced substantially only when an inducer which corresponds to the promoter is present.

A “constitutive” promoter is a nucleotide sequence which, when operably linked with a polynucleotide which encodes or specifies a gene product, causes the gene product to be produced in a cell under most or all physiological conditions of the cell.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “RNA” as used herein is defined as ribonucleic acid.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide may serve a non-coding function (e.g., promoter, origin of replication, ribosome-binding site, etc.) as well.

The term “recombinant polypeptide” as used herein is defined as a polypeptide produced by using recombinant DNA methods.

As used herein, “Transcription Activator-Like Effector Nucleases (TALENs)” are artificial restriction enzymes generated by fusing the TAL effector DNA binding domain to a DNA cleavage domain. These reagents enable efficient, programmable, and specific DNA cleavage and represent powerful tools for editing genetic material in situ. Transcription activator-like effectors (TALEs) can be quickly engineered to bind practically any DNA sequence. The term TALEN, as used herein, is broad and includes a monomeric TALEN that can cleave double stranded DNA without assistance from another TALEN. The term TALEN is also used to refer to one or both members of a pair of TALENs that are engineered to work together to cleave DNA at the same site. TALENs that work together may be referred to as a left-TALEN and a right-TALEN, which references the handedness of DNA. See U.S. Ser. No. 12/965,590; U.S. Ser. No. 13/426,991 (U.S. Pat. No. 8,450,471); U.S. Ser. No. 13/427,040 (U.S. Pat. No. 8,440,431); U.S. Ser. No. 13/427,137 (U.S. Pat. No. 8,440,432); and U.S. Ser. No. 13/738,381, all of which are incorporated by reference herein in their entirety.

“Variant” as the term is used herein, is a nucleic acid sequence or a peptide sequence that differs in sequence from a reference nucleic acid sequence or peptide sequence respectively, but retains essential biological properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide can differ in amino acid sequence by one or more substitutions, additions, deletions in any combination. A variant of a nucleic acid or peptide can be a naturally occurring such as an allelic variant, or can be a variant that is not known to occur naturally. Non-naturally occurring variants of nucleic acids and peptides may be made by mutagenesis techniques or by direct synthesis.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Fusion Proteins

In one aspect, the present invention is based on the development of novel fusions of editing proteins which are effectively delivered to the nucleus. In one aspect, the invention provides fusion proteins comprising an editing protein and a nuclear localization signal (NLS) having a second amino acid sequence.

In one embodiment, the editing protein includes, but is not limited to, a CRISPR-associated (Cas) protein, transcription activator-like effector-based nuclease (TALEN) protein, a zinc finger nuclease (ZFN) protein, and a protein having a DNA binding domain.

Non-limiting examples of Cas proteins include Cas1, Cas1B, Cas2, Cas3, Cas4, Cas5, Cas6, Cas7, Cas8, Cas9, Cas10, Csy1, Csy2, Csy3, Cse1, Cse2, Csc1, Csc2, Csa5, Csn2. Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, SpCas9, StCas9, NmCas9, SaCas9, CjCas9, CjCas9, AsCpf1, LbCpf1, FnCpf1, VRER SpCas9, VQR SpCas9, xCas9 3.7, homologs thereof, orthologs thereof, or modified versions thereof. In some embodiments, the Cas protein has DNA or RNA cleavage activity. In some embodiments, the Cas protein directs cleavage of one or both strands of a nucleic acid molecule at the location of a target sequence, such as within the target sequence and/or within the complement of the target sequence. In some embodiments, the Cas protein directs cleavage of one or both strands within about 1, 2, 3, 4, 5, 6, 7, 8, 9, 10, 15, 20, 25, 50, 100, 200, 500, or more base pairs from the first or last nucleotide of a target sequence. In one embodiment, the Cas protein is Cas9, Cas13, or Cpf1. In one embodiment, Cas protein is Cas9. In one embodiment, Cas protein is catalytically deficient (dCas).

In one embodiment, the Cas protein comprises a sequence at least 70%, at least 71%, at least 72%, at least 73%, at least 74%, at least 75%, at least 76%, at least 77%, at least 78%, at least 79%, 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% identical to one of SEQ ID NOs:41-46. In one embodiment, the Cas protein comprises a sequence of one of SEQ ID NOs:41-46.

In one embodiment, the NLS is a retrotransposon NLS. In one embodiment, the NLS is derived from Ty1, yeast GAL4, SKI3, L29 or histone H2B proteins, polyoma virus large T protein, VP1 or VP2 capsid protein, SV40 VP1 or VP2 capsid protein, Adenovirus El a or DBP protein, influenza virus NS1 protein, hepatitis vims core antigen or the mammalian lamin, c-myc, max, c-myb, p53, c-erbA, jun, Tax, steroid receptor or Mx proteins, Nucleoplasmin (NPM2), Nucleophosmin (NPM1), or simian vims 40 (“SV40”) T-antigen. In one embodiment, the NLS is a Ty1 or Ty1-derived NLS, a Ty2 or Ty2-derived NLS or a MAK11 or MAK11-derived NLS. In one embodiment, the Ty1 NLS comprises an amino acid sequence of SEQ ID NO:51. In one embodiment, the Ty2 NLS comprises an amino acid sequence of SEQ ID NO:254. In one embodiment, the MAK11 NLS comprises an amino acid sequence of SEQ ID NO:256. In one embodiment, the NLS comprises a sequence at least 70%, at least 71%, at least 72%, at least 73%, at least 74%, at least 75%, at least 76%, at least 77%, at least 78%, at least 79%, 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% identical to one of SEQ ID NOs:47-56 and 254-257. In one embodiment, the NLS protein comprises a sequence of one of SEQ ID NOs: 47-56 and 254-257.

In one embodiment, the NLS is a Ty1-like NLS. For example, in one embodiment, the Ty-like NLS comprises KKRX motif. In one embodiment, the Ty1-like NLS comprises KKRX motif at the N-terminal end. In one embodiment, the Ty1-like NLS comprises KKR motif. In one embodiment, the Ty1-like NLS comprises KKR motif at the C-terminal end. In one embodiment, the Ty1-like NLS comprises a KKRX and a KKR motif. In one embodiment, the Ty1-like NLS comprises a KKRX at the N-terminal end and a KKR motif at the C-terminal end. In one embodiment, the Ty1-like NLS comprises at least 20 amino acids. In one embodiment, the Ty1-like NLS comprises between 20 and 40 amino acids. In one embodiment, the Ty1-like NLS comprises a sequence at least 70%, at least 71%, at least 72%, at least 73%, at least 74%, at least 75%, at least 76%, at least 77%, at least 78%, at least 79%, 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% identical to one of SEQ ID NOs:275-887. In one embodiment, the Ty1-like NLS protein comprises a sequence of one of SEQ ID NOs:275-887.

In one embodiment, the fusion protein comprises a sequence at least 70%, at least 71%, at least 72%, at least 73%, at least 74%, at least 75%, at least 76%, at least 77%, at least 78%, at least 79%, 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% identical to one of SEQ ID NOs:249-250. In one embodiment, the fusion protein comprises a sequence of one of SEQ ID NOs:249-250.

In one aspect, the present invention is based on the development of novel fusions of editing proteins and retroviral integrase proteins which are effectively delivered to the nucleus. These fusion proteins combine the DNA integration activity of viral integrase and the programmable DNA targeting capability of catalytically dead Cas. Thus, since this fusion protein does not rely on cellular pathways for DNA insertion, or require cellular energy source, such as ATP, this enzyme can work in many contexts, such as from in vitro, to prokaryotic cells, to dividing or non-dividing eukaryotic cells. Further, because integrase does not require regions of homology for insertion, only small terminal motif sequences specific to each integrase family, these fusion proteins editing can utilize a single DNA donor template for multiplex genome integration, if guided by multiple guide-RNAs.

Thus, in one aspect, the present invention provides fusion proteins comprising a CRISPR-associated (Cas) protein having a first amino acid sequence, a nuclear localization signal (NLS) having a second amino acid sequence, and a retroviral integrase (IN) or a fragment or variant thereof having a third amino acid sequence.

In one embodiment, the retroviral IN is human immunodeficiency virus (HIV) IN, Rous sarcoma virus (RSV) IN, Mouse mammary tumor virus (MMTV) IN, Moloney murine leukemia virus (MoLV) IN, bovine leukemia virus (BLV) IN, Human T-lymphotropic virus (HTLV) IN, avian sarcoma leukosis virus (ASLV) IN, feline leukemia virus (FLV) IN, xenotropic murine leukemia virus-related virus (XMLV) IN, simian immunodeficiency virus (SIV) IN, feline immunodeficiency virus (FIV) IN, equine infectious anemia virus (EIAV) IN, Prototype foamy virus (PFV) IN, simian foamy virus (SFV) IN, human foamy virus (HFV) IN, walleye dermal sarcoma virus (WDSV) IN, or bovine immunodeficiency virus (BIV) IN.

In one embodiment, the integrase is a retrotransposon integrase. In one embodiment, the retrotransposon integrase is Ty1, or Ty2. In one embodiment, the integrase is a bacterial integrase. In one embodiment, the bacterial integrase is insF.

In one embodiment, the retroviral IN is HIV IN. In one embodiment, the HIV IN comprises one or more amino acid substitutions, wherein the substitution improves catalytic activity, improves solubility, or increases interaction with one or more host cellular cofactors. In one embodiment, HIV IN comprises one or more, two or more, three or more, four or more, five or more, six or more, seven or more, eight or more or nine amino acid substitutions selected from the group consisting of E85G, E85F, D116N, F185K, C280S, T97A, Y134R, G140S, and Q148H. In one embodiment, HIV IN comprises amino acid substitutions F185K and C280S. In one embodiment, HIV IN comprises amino acid substitutions T97A and Y134R. In one embodiment, HIV IN comprises amino acid substitutions G140S and Q148H.

In one embodiment, the retroviral IN fragment comprises the IN N-terminal domain (NTD), and the IN catalytic core domain (CCD). In one embodiment, the retroviral IN fragment comprises the IN CCD and the IN C-terminal domain (CTD). In one embodiment, the retroviral IN fragment comprises the IN NTD. In one embodiment, the retroviral IN fragment comprises the IN CCD. In one embodiment, the retroviral IN fragment comprises the IN CTD. The in one embodiment, the fragments of the integrase retain at least one activity of the full length integrase. Retroviral integrase functions and fragments are known in the art and can be found in, for example, Li, et al., 2011, Virology 411:194-205, and Maertens et al., 2010, Nature 468:326-29, which are incorporated by reference herein.

In one embodiment, the retroviral IN comprises a sequence at least 70%, at least 71%, at least 72%, at least 73%, at least 74%, at least 75%, at least 76%, at least 77%, at least 78%, at least 79%, 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% identical to one of SEQ ID NOs:1-40. In one embodiment, the retroviral IN comprises a sequence of one of SEQ ID NOs:1-40.

In some embodiments, the CRISPR-Cas domain comprises a Cas protein. Non-limiting examples of Cas proteins include Cas1, Cas1B, Cas2, Cas3, Cas4, Cas5, Cas6, Cas7, Cas8, Cas9, Cas10, Csy1, Csy2, Csy3, Cse1, Cse2, Csc1, Csc2, Csa5, Csn2. Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, SpCas9, StCas9, NmCas9, SaCas9, CjCas9, CjCas9, AsCpf1, LbCpf1, FnCpf1, VRER SpCas9, VQR SpCas9, xCas9 3.7, homologs thereof, orthologs thereof, or modified versions thereof. In some embodiments, the Cas protein has DNA or RNA cleavage activity. In some embodiments, the Cas protein directs cleavage of one or both strands of a nucleic acid molecule at the location of a target sequence, such as within the target sequence and/or within the complement of the target sequence. In some embodiments, the Cas protein directs cleavage of one or both strands within about 1, 2, 3, 4, 5, 6, 7, 8, 9, 10, 15, 20, 25, 50, 100, 200, 500, or more base pairs from the first or last nucleotide of a target sequence. In one embodiment, the Cas protein is Cas9, Cas13, or Cpf1. In one embodiment, Cas protein is catalytically deficient (dCas).

In one embodiment, the Cas protein comprises a sequence at least 70%, at least 71%, at least 72%, at least 73%, at least 74%, at least 75%, at least 76%, at least 77%, at least 78%, at least 79%, 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% identical to one of SEQ ID NOs:41-46. In one embodiment, the Cas protein comprises a sequence of one of SEQ ID NOs:41-46.

In one embodiment, the NLS is a retrotransposon NLS. In one embodiment, the NLS is derived from Ty1, yeast GAL4, SKI3, L29 or histone H2B proteins, polyoma virus large T protein, VP1 or VP2 capsid protein, SV40 VP1 or VP2 capsid protein, Adenovirus El a or DBP protein, influenza virus NS1 protein, hepatitis vims core antigen or the mammalian lamin, c-myc, max, c-myb, p53, c-erbA, jun, Tax, steroid receptor or Mx proteins, Nucleoplasmin (NPM2), Nucleophosmin (NPM1), or simian vims 40 (“SV40”) T-antigen. In one embodiment, the NLS is a Ty1 or Ty1-derived NLS, a Ty2 or Ty2-derived NLS or a MAK11 or MAK11-derived NLS. In one embodiment, the Ty1 NLS comprises an amino acid sequence of SEQ ID NO:51. In one embodiment, the Ty2 NLS comprises an amino acid sequence of SEQ ID NO:254. In one embodiment, the MAK11 NLS comprises an amino acid sequence of SEQ ID NO:256. In one embodiment, the NLS comprises a sequence at least 70%, at least 71%, at least 72%, at least 73%, at least 74%, at least 75%, at least 76%, at least 77%, at least 78%, at least 79%, 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% identical to one of SEQ ID NOs:47-56 and 254-257. In one embodiment, the NLS protein comprises a sequence of one of SEQ ID NOs: 47-56 and 254-257.

In one embodiment, the NLS is a Ty1-like NLS. For example, in one embodiment, the Ty-like NLS comprises KKRX motif. In one embodiment, the Ty1-like NLS comprises KKRX motif at the N-terminal end. In one embodiment, the Ty1-like NLS comprises KKR motif. In one embodiment, the Ty1-like NLS comprises KKR motif at the C-terminal end. In one embodiment, the Ty1-like NLS comprises a KKRX and a KKR motif. In one embodiment, the Ty1-like NLS comprises a KKRX at the N-terminal end and a KKR motif at the C-terminal end. In one embodiment, the Ty1-like NLS comprises at least 20 amino acids. In one embodiment, the Ty1-like NLS comprises between 20 and 40 amino acids. In one embodiment, the Ty1-like NLS comprises a sequence at least 70%, at least 71%, at least 72%, at least 73%, at least 74%, at least 75%, at least 76%, at least 77%, at least 78%, at least 79%, 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% identical to one of SEQ ID NOs: 275-887. In one embodiment, the Ty1-like NLS protein comprises a sequence of one of SEQ ID NOs: 275-887.

In one embodiment, the fusion protein comprises a sequence at least 70%, at least 71%, at least 72%, at least 73%, at least 74%, at least 75%, at least 76%, at least 77%, at least 78%, at least 79%, 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% identical to one of SEQ ID NOs:249-250. In one embodiment, the fusion protein comprises a sequence of one of SEQ ID NOs:249-250.

In one embodiment, the NLS comprises a combination of two distinct NLS. For example, in one embodiment, the NLS comprises a Ty1-derived NLS and a SV40-derived NLS. In one embodiment, the NLS is a Ty1 or Ty1-derived NLS, a Ty2 or Ty2-derived NLS or a MAK11 or MAK11-derived NLS. In one embodiment, the Ty1 NLS comprises an amino acid sequence of SEQ ID NO:51. In one embodiment, the Ty2 NLS comprises an amino acid sequence of SEQ ID NO:254. In one embodiment, the MAK11 NLS comprises an amino acid sequence of SEQ ID NO:256.

In one embodiment, the NLS comprises two copies of the same NLS. For example, in one embodiment, the NLS comprises a multimer of a first Ty1-derived NLS and a second Ty1-derived NLS.

In one embodiment, the NLS comprises a first sequence at least 70%, at least 71%, at least 72%, at least 73%, at least 74%, at least 75%, at least 76%, at least 77%, at least 78%, at least 79%, 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% to one of SEQ ID NOs:47-56, 254-257, and 275-887, and a second a sequence at least 70%, at least 71%, at least 72%, at least 73%, at least 74%, at least 75%, at least 76%, at least 77%, at least 78%, at least 79%, 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% to one of SEQ ID NOs:47-56, 254-257, and 275-887. In one embodiment, the first sequence and second sequence are the same. In one embodiment, the first sequence and second sequence are different.

In one embodiment, the fusion protein comprises a sequence 70%, at least 71%, at least 72%, at least 73%, at least 74%, at least 75%, at least 76%, at least 77%, at least 78%, at least 79%, 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% to one of SEQ ID NOs:57-98. In one embodiment, the fusion protein comprises a sequence of one of SEQ ID NOs:57-98.

The peptide of the present invention may be made using chemical methods. For example, peptides can be synthesized by solid phase techniques (Roberge J Y et al (1995) Science 269: 202-204), cleaved from the resin, and purified by preparative high-performance liquid chromatography. Automated synthesis may be achieved, for example, using the ABI 431 A Peptide Synthesizer (Perkin Elmer) in accordance with the instructions provided by the manufacturer.

The invention should also be construed to include any form of a peptide having substantial homology to a fusion-protein disclosed herein. In one embodiment, a peptide which is “substantially homologous” is about 50% homologous, about 70% homologous, about 80% homologous, about 90% homologous, about 95% homologous, or about 99% homologous to amino acid sequence of a fusion-protein disclosed herein.

The peptide may alternatively be made by recombinant means or by cleavage from a longer polypeptide. The composition of a peptide may be confirmed by amino acid analysis or sequencing.

The variants of the peptides according to the present invention may be (i) one in which one or more of the amino acid residues are substituted with a conserved or non-conserved amino acid residue and such substituted amino acid residue may or may not be one encoded by the genetic code, (ii) one in which there are one or more modified amino acid residues, e.g., residues that are modified by the attachment of substituent groups, (iii) one in which the peptide is an alternative splice variant of the peptide of the present invention, (iv) fragments of the peptides and/or (v) one in which the peptide is fused with another peptide, such as a leader or secretory sequence or a sequence which is employed for purification (for example, His-tag) or for detection (for example, Sv5 epitope tag). The fragments include peptides generated via proteolytic cleavage (including multi-site proteolysis) of an original sequence. Variants may be post-translationally, or chemically modified. Such variants are deemed to be within the scope of those skilled in the art from the teaching herein.

As known in the art the “similarity” between two peptides is determined by comparing the amino acid sequence and its conserved amino acid substitutes of one polypeptide to a sequence of a second polypeptide. Variants are defined to include peptide sequences different from the original sequence. In one embodiment, variants are different from the original sequence in less than 40% of residues per segment of interest different from the original sequence in less than 25% of residues per segment of interest, different by less than 10% of residues per segment of interest, or different from the original protein sequence in just a few residues per segment of interest and at the same time sufficiently homologous to the original sequence to preserve the functionality of the original sequence and/or the ability to stimulate the differentiation of a stem cell into the osteoblast lineage. The present invention includes amino acid sequences that are at least 60%, 65%, 70%, 72%, 74%, 76%, 78%, 80%, 90%, or 95% similar or identical to the original amino acid sequence. The degree of identity between two peptides is determined using computer algorithms and methods that are widely known for the persons skilled in the art. The identity between two amino acid sequences may be determined by using the BLASTP algorithm [BLAST Manual, Altschul, S., et al., NCBI NLM NIH Bethesda, Md. 20894, Altschul, S., et al., J. Mol. Biol. 215: 403-410 (1990)].

The peptides of the invention can be post-translationally modified. For example, post-translational modifications that fall within the scope of the present invention include signal peptide cleavage, glycosylation, acetylation, isoprenylation, proteolysis, myristoylation, protein folding and proteolytic processing, etc. Some modifications or processing events require introduction of additional biological machinery. For example, processing events, such as signal peptide cleavage and core glycosylation, are examined by adding canine microsomal membranes or Xenopus egg extracts (U.S. Pat. No. 6,103,489) to a standard translation reaction.

The peptides of the invention may include unnatural amino acids formed by post-translational modification or by introducing unnatural amino acids during translation. A variety of approaches are available for introducing unnatural amino acids during protein translation.

A peptide or protein of the invention may be phosphorylated using conventional methods such as the method described in Reedijk et al. (The EMBO Journal 11(4):1365, 1992).

Cyclic derivatives of the peptides of the invention are also part of the present invention. Cyclization may allow the peptide to assume a more favorable conformation for association with other molecules. Cyclization may be achieved using techniques known in the art. For example, disulfide bonds may be formed between two appropriately spaced components having free sulfhydryl groups, or an amide bond may be formed between an amino group of one component and a carboxyl group of another component. Cyclization may also be achieved using an azobenzene-containing amino acid as described by Ulysse, L., et al., J. Am. Chem. Soc. 1995, 117, 8466-8467. The components that form the bonds may be side chains of amino acids, non-amino acid components or a combination of the two. In an embodiment of the invention, cyclic peptides may comprise a beta-turn in the right position. Beta-turns may be introduced into the peptides of the invention by adding the amino acids Pro-Gly at the right position.

It may be desirable to produce a cyclic peptide which is more flexible than the cyclic peptides containing peptide bond linkages as described above. A more flexible peptide may be prepared by introducing cysteines at the right and left position of the peptide and forming a disulphide bridge between the two cysteines. The two cysteines are arranged so as not to deform the beta-sheet and turn. The peptide is more flexible as a result of the length of the disulfide linkage and the smaller number of hydrogen bonds in the beta-sheet portion. The relative flexibility of a cyclic peptide can be determined by molecular dynamics simulations.

The invention also relates to peptides comprising an IN-Cas9 peptide fused to, or integrated into, a target protein, and/or a targeting domain capable of directing the chimeric protein to a desired cellular component or cell type or tissue. The chimeric proteins may also contain additional amino acid sequences or domains. The chimeric proteins are recombinant in the sense that the various components are from different sources, and as such are not found together in nature (i.e., are heterologous).

In one embodiment, the targeting domain can be a membrane spanning domain, a membrane binding domain, or a sequence directing the protein to associate with for example vesicles or with the nucleus. In one embodiment, the targeting domain can target a peptide to a particular cell type or tissue. For example, the targeting domain can be a cell surface ligand or an antibody against cell surface antigens of a target tissue. A targeting domain may target the peptide of the invention to a cellular component.

A peptide of the invention may be synthesized by conventional techniques. For example, the peptides or chimeric proteins may be synthesized by chemical synthesis using solid phase peptide synthesis. These methods employ either solid or solution phase synthesis methods (see for example, J. M. Stewart, and J. D. Young, Solid Phase Peptide Synthesis, 2^{nd} Ed., Pierce Chemical Co., Rockford Ill. (1984) and G. Barany and R. B. Merrifield, The Peptides: Analysis Synthesis, Biology editors E. Gross and J. Meienhofer Vol. 2 Academic Press, New York, 1980, pp. 3-254 for solid phase synthesis techniques; and M Bodansky, Principles of Peptide Synthesis, Springer-Verlag, Berlin 1984, and E. Gross and J. Meienhofer, Eds., The Peptides: Analysis, Synthesis, Biology, suprs, Vol 1, for classical solution synthesis). By way of example, a peptide of the invention may be synthesized using 9-fluorenyl methoxycarbonyl (Fmoc) solid phase chemistry with direct incorporation of phosphothreonine as the N-fluorenylmethoxy-carbonyl-O-benzyl-L-phosphothreonine derivative.

N-terminal or C-terminal fusion proteins comprising a peptide or chimeric protein of the invention conjugated with other molecules may be prepared by fusing, through recombinant techniques, the N-terminal or C-terminal of the peptide or chimeric protein, and the sequence of a selected protein or selectable marker with a desired biological function. The resultant fusion proteins contain the IN-Cas9 peptide fused to the selected protein or marker protein as described herein. Examples of proteins which may be used to prepare fusion proteins include immunoglobulins, glutathione-S-transferase (GST), hemagglutinin (HA), and truncated myc.

Peptides of the invention may be developed using a biological expression system. The use of these systems allows the production of large libraries of random peptide sequences and the screening of these libraries for peptide sequences that bind to particular proteins. Libraries may be produced by cloning synthetic DNA that encodes random peptide sequences into appropriate expression vectors (see Christian et al 1992, J. Mol. Biol. 227:711; Devlin et al, 1990 Science 249:404; Cwirla et al 1990, Proc. Natl. Acad, Sci. USA, 87:6378). Libraries may also be constructed by concurrent synthesis of overlapping peptides (see U.S. Pat. No. 4,708,871).

The peptides and chimeric proteins of the invention may be converted into pharmaceutical salts by reacting with inorganic acids such as hydrochloric acid, sulfuric acid, hydrobromic acid, phosphoric acid, etc., or organic acids such as formic acid, acetic acid, propionic acid, glycolic acid, lactic acid, pyruvic acid, oxalic acid, succinic acid, malic acid, tartaric acid, citric acid, benzoic acid, salicylic acid, benezenesulfonic acid, and toluenesulfonic acids.

### Nucleic Acids

In one embodiment, the present invention a nucleic acid molecule encoding a fusion protein. In one embodiment, the nucleic acid molecule comprises a first nucleic acid sequence encoding an editing protein; and a second nucleic acid sequence encoding a nuclear localization signal (NLS).

In one embodiment, the editing protein includes, but is not limited to, a CRISPR-associated (Cas) protein, transcription activator-like effector-based nuclease (TALEN) protein, a zinc finger nuclease (ZFN) protein, and a protein having a DNA binding domain. In one embodiment, the editing protein is a Cas protein.

Non-limiting examples of Cas proteins include Cas1, Cas1B, Cas2, Cas3, Cas4, Cas5, Cas6, Cas7, Cas8, Cas9, Cas10, Csy1, Csy2, Csy3, Cse1, Cse2, Csc1, Csc2, Csa5, Csn2. Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, SpCas9, StCas9, NmCas9, SaCas9, CjCas9, CjCas9, AsCpf1, LbCpf1, FnCpf1, VRER SpCas9, VQR SpCas9, xCas9 3.7, homologs thereof, orthologs thereof, or modified versions thereof. In some embodiments, the Cas protein has DNA or RNA cleavage activity. In some embodiments, the Cas protein directs cleavage of one or both strands of a nucleic acid molecule at the location of a target sequence, such as within the target sequence and/or within the complement of the target sequence. In some embodiments, the Cas protein directs cleavage of one or both strands within about 1, 2, 3, 4, 5, 6, 7, 8, 9, 10, 15, 20, 25, 50, 100, 200, 500, or more base pairs from the first or last nucleotide of a target sequence. In one embodiment, the Cas protein is Cas9, Cas13, or Cpf1. In one embodiment, Cas protein is Cas9. In one embodiment, Cas protein is catalytically deficient (dCas).

In one embodiment, the first nucleic acid sequence encoding a Cas protein comprises a nucleic acid sequence encoding an amino acid sequence at least 70%, at least 71%, at least 72%, at least 73%, at least 74%, at least 75%, at least 76%, at least 77%, at least 78%, at least 79%, 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% identical to one of SEQ ID NOs:41-46. In one embodiment, the first nucleic acid sequence encoding a Cas protein comprises a nucleic acid sequence encoding one of SEQ ID NOs:41-46.

In one embodiment, the first nucleic acid sequence encoding a Cas protein comprises a nucleic acid sequence at least 70%, at least 71%, at least 72%, at least 73%, at least 74%, at least 75%, at least 76%, at least 77%, at least 78%, at least 79%, 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% identical to one of SEQ ID NOs:139-144. In one embodiment, the first nucleic acid sequence encoding a Cas protein comprises a nucleic acid sequence of one of SEQ ID NOs:139-144.

In one embodiment, the second nucleic acid sequence encodes a nuclear localization signal (NLS). In one embodiment, the NLS is a retrotransposon NLS. In one embodiment, the NLS is derived from yeast GAL4, SKI3, L29 or histone H2B proteins, polyoma virus large T protein, VP1 or VP2 capsid protein, SV40 VP1 or VP2 capsid protein, Adenovirus El a or DBP protein, influenza virus NS1 protein, hepatitis vims core antigen or the mammalian lamin, c-myc, max, c-myb, p53, c-erbA, jun, Tax, steroid receptor or Mx proteins, Nucleoplasmin (NPM2), Nucleophosmin (NPM1), or simian vims 40 (“SV40”) T-antigen. In one embodiment, the NLS is a Ty1 or Ty1-derived NLS, a Ty2 or Ty2-derived NLS or a MAK11 or MAK11-derived NLS. In one embodiment, the Ty1 NLS comprises an amino acid sequence of SEQ ID NO:51. In one embodiment, the Ty2 NLS comprises an amino acid sequence of SEQ ID NO:254. In one embodiment, the MAK11 NLS comprises an amino acid sequence of SEQ ID NO:256.

In one embodiment, the NLS is a Ty1-like NLS. For example, in one embodiment, the Ty-like NLS comprises KKRX motif. In one embodiment, the Ty1-like NLS comprises KKRX motif at the N-terminal end. In one embodiment, the Ty1-like NLS comprises KKR motif. In one embodiment, the Ty1-like NLS comprises KKR motif at the C-terminal end. In one embodiment, the Ty1-like NLS comprises a KKRX and a KKR motif. In one embodiment, the Ty1-like NLS comprises a KKRX at the N-terminal end and a KKR motif at the C-terminal end. In one embodiment, the Ty1-like NLS comprises at least 20 amino acids. In one embodiment, the Ty1-like NLS comprises between 20 and 40 amino acids.

In one embodiment, the retrotransposon NLS increases nuclear localization. In one embodiment, the retrotransposon NLS increases nuclear localization significantly more compared to non-retrotransposon NLS.

In one embodiment, second nucleic acid sequence encoding a NLS comprises a nucleic acid sequence encoding an amino acid sequence at least 70%, at least 71%, at least 72%, at least 73%, at least 74%, at least 75%, at least 76%, at least 77%, at least 78%, at least 79%, 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% identical to one of SEQ ID NOs:47-56, 254-257, and 275-887. In one embodiment, second nucleic acid sequence encoding a NLS comprises a nucleic acid sequence encoding one of SEQ ID NOs:47-56, 254-257, and 275-887.

In one embodiment, second nucleic acid sequence encoding a NLS comprises a nucleic acid sequence at least 70%, at least 71%, at least 72%, at least 73%, at least 74%, at least 75%, at least 76%, at least 77%, at least 78%, at least 79%, 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% identical to one of SEQ ID NOs:145-154. In one embodiment, second nucleic acid sequence encoding a NLS comprises a nucleic acid sequence of one of SEQ ID NOs:145-154.

In one embodiment, the nucleic acid molecule encodes a fusion protein comprising a sequence at least 70%, at least 71%, at least 72%, at least 73%, at least 74%, at least 75%, at least 76%, at least 77%, at least 78%, at least 79%, 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% identical to one of SEQ ID NOs:249-250. In one embodiment, the nucleic acid molecule encodes a fusion protein comprising a sequence of one of SEQ ID NOs:249-250.

In one embodiment, the nucleic acid molecule comprises; a first nucleic acid sequence encoding an editing protein; a second nucleic acid sequence encoding a nuclear localization signal (NLS); and a third nucleic acid sequence encoding a retroviral integrase (IN) or a fragment thereof.

In one embodiment, the retroviral IN is human immunodeficiency virus (HIV) IN, Rous sarcoma virus (RSV) IN, Mouse mammary tumor virus (MMTV) IN, Moloney murine leukemia virus (MoLV) IN, bovine leukemia virus (BLV) IN, Human T-lymphotropic virus (HTLV) IN, avian sarcoma leukosis virus (ASLV) IN, feline leukemia virus (FLV) IN, xenotropic murine leukemia virus-related virus (XMLV) IN, simian immunodeficiency virus (SIV) IN, feline immunodeficiency virus (FIV) IN, equine infectious anemia virus (EIAV) IN, Prototype foamy virus (PFV) IN, simian foamy virus (SFV) IN, human foamy virus (HFV) IN, walleye dermal sarcoma virus (WDSV) IN, or bovine immunodeficiency virus (BIV) IN.

In one embodiment, the retroviral IN is HIV IN. In one embodiment, the HIV IN comprises one or more amino acid substitutions, wherein the substitution improves catalytic activity, improves solubility, or increases interaction with one or more host cellular cofactors. In one embodiment, HIV IN comprises one or more, two or more, three or more, four or more, five or more, six or more, seven or more, eight or more or nine amino acid substitutions selected from the group consisting of E85G, E85F, D116N, F185K, C280S, T97A, Y134R, G140S, and Q148H. In one embodiment, HIV IN comprises amino acid substitutions F185K and C280S. In one embodiment, HIV IN comprises amino acid substitutions T97A and Y134R. In one embodiment, HIV IN comprises amino acid substitutions G140S and Q148H.

In one embodiment, the retroviral IN fragment comprises the IN N-terminal domain (NTD), and the IN catalytic core domain (CCD). In one embodiment, the retroviral IN fragment comprises the IN CCD and the IN C-terminal domain (CTD). In one embodiment, the retroviral IN fragment comprises the IN NTD. In one embodiment, the retroviral IN fragment comprises the IN CCD. In one embodiment, the retroviral IN fragment comprises the IN CTD. The in one embodiment, the fragments of the integrase retain at least one activity of the full length integrase. Retroviral integrase functions and fragments are known in the art and can be found in, for example, Li, et al., 2011, Virology 411:194-205, and Maertens et al., 2010, Nature 468:326-29, which are incorporated by reference herein.

In one embodiment, the third nucleic acid sequence encoding a retroviral IN comprises a nucleic acid sequence encoding an amino acid sequence at least 70%, at least 71%, at least 72%, at least 73%, at least 74%, at least 75%, at least 76%, at least 77%, at least 78%, at least 79%, 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% identical to one of SEQ ID NOs:1-40. In one embodiment, the third nucleic acid sequence encoding a retroviral IN comprises a nucleic acid sequence encoding one of SEQ ID NOs:1-40.

In one embodiment, the third nucleic acid sequence encoding a retroviral IN comprises a nucleic acid sequence at least at least 70%, at least 71%, at least 72%, at least 73%, at least 74%, at least 75%, at least 76%, at least 77%, at least 78%, at least 79%, 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% identical to one of SEQ ID NOs:99-138. In one embodiment, the third nucleic acid sequence encoding a retroviral IN comprises a nucleic acid sequence of one of SEQ ID NOs:99-138.

In one embodiment, the editing protein includes, but is not limited to, a CRISPR-associated (Cas) protein, transcription activator-like effector-based nuclease (TALEN) protein, a zinc finger nuclease (ZFN) protein, and a DNA-binding protein. In one embodiment, the editing protein is a Cas protein. In one embodiment, the Cas protein is Cas9, Cas13, or Cpf1. In one embodiment, the Cas protein is catalytically deficient (dCas).

In one embodiment, the first nucleic acid sequence encodes a Cas protein. In one embodiment, the first nucleic acid sequence encoding a Cas protein comprises a nucleic acid sequence encoding an amino acid sequence at least 70%, at least 71%, at least 72%, at least 73%, at least 74%, at least 75%, at least 76%, at least 77%, at least 78%, at least 79%, 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% identical to one of SEQ ID NOs:41-46. In one embodiment, the first nucleic acid sequence encoding a Cas protein comprises a nucleic acid sequence encoding one of SEQ ID NOs:41-46.

In one embodiment, the first nucleic acid sequence encoding a Cas protein comprises a nucleic acid sequence at least 70%, at least 71%, at least 72%, at least 73%, at least 74%, at least 75%, at least 76%, at least 77%, at least 78%, at least 79%, 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% identical to one of SEQ ID NOs:139-144. In one embodiment, the first nucleic acid sequence encoding a Cas protein comprises a nucleic acid sequence of one of SEQ ID NOs:139-144.

In one embodiment, the second nucleic acid sequence encodes a nuclear localization signal (NLS). In one embodiment, the NLS is a retrotransposon NLS. In one embodiment, the NLS is derived from yeast GAL4, SKI3, L29 or histone H2B proteins, polyoma virus large T protein, VP1 or VP2 capsid protein, SV40 VP1 or VP2 capsid protein, Adenovirus El a or DBP protein, influenza virus NS1 protein, hepatitis vims core antigen or the mammalian lamin, c-myc, max, c-myb, p53, c-erbA, jun, Tax, steroid receptor or Mx proteins, Nucleoplasmin (NPM2), Nucleophosmin (NPM1), or simian vims 40 (“SV40”) T-antigen. In one embodiment, the NLS is a Ty1 or Ty1-derived NLS, a Ty2 or Ty2-derived NLS or a MAK11 or MAK11-derived NLS. In one embodiment, the Ty1 NLS comprises an amino acid sequence of SEQ ID NO:51. In one embodiment, the Ty2 NLS comprises an amino acid sequence of SEQ ID NO:254. In one embodiment, the MAK11 NLS comprises an amino acid sequence of SEQ ID NO:256.

In one embodiment, the retrotransposon NLS increases nuclear localization. In one embodiment, the retrotransposon NLS increases nuclear localization significantly more compared to non-retrotransposon NLS.

In one embodiment, second nucleic acid sequence encoding a NLS comprises a nucleic acid sequence encoding an amino acid sequence at least 70%, at least 71%, at least 72%, at least 73%, at least 74%, at least 75%, at least 76%, at least 77%, at least 78%, at least 79%, 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% identical to one of SEQ ID NOs:47-56, 254-257 and 275-87. In one embodiment, second nucleic acid sequence encoding a NLS comprises a nucleic acid sequence encoding one of SEQ ID NOs: 47-56, 254-257 and 275-887.

In one embodiment, second nucleic acid sequence encoding a NLS comprises a nucleic acid sequence at least 70%, at least 71%, at least 72%, at least 73%, at least 74%, at least 75%, at least 76%, at least 77%, at least 78%, at least 79%, 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% identical to one of SEQ ID NOs:145-154. In one embodiment, second nucleic acid sequence encoding a NLS comprises a nucleic acid sequence of one of SEQ ID NOs:145-154.

In one embodiment, the nucleic acid molecule encodes a fusion protein comprising a sequence at least 70%, at least 71%, at least 72%, at least 73%, at least 74%, at least 75%, at least 76%, at least 77%, at least 78%, at least 79%, 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% identical to one of SEQ ID NOs:57-98. In one embodiment, the nucleic acid molecule encodes a fusion protein comprising a sequence of one of SEQ ID NOs:57-98.

In one embodiment, the nucleic acid molecule comprises a nucleic acid sequence at least 70%, at least 71%, at least 72%, at least 73%, at least 74%, at least 75%, at least 76%, at least 77%, at least 78%, at least 79%, 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% identical to one of SEQ ID NOs:155-196. In one embodiment, the nucleic acid molecule comprises a nucleic acid sequence of one of SEQ ID NOs:155-196.

The isolated nucleic acid sequence encoding a fusion protein can be obtained using any of the many recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

The isolated nucleic acid may comprise any type of nucleic acid, including, but not limited to DNA and RNA. For example, in one embodiment, the composition comprises an isolated DNA molecule, including for example, an isolated cDNA molecule, encoding a fusion protein of the invention. In one embodiment, the composition comprises an isolated RNA molecule encoding a fusion protein of the invention, or a functional fragment thereof.

The nucleic acid molecules of the present invention can be modified to improve stability in serum or in growth medium for cell cultures. Modifications can be added to enhance stability, functionality, and/or specificity and to minimize immunostimulatory properties of the nucleic acid molecule of the invention. For example, in order to enhance the stability, the 3′-residues may be stabilized against degradation, e.g., they may be selected such that they consist of purine nucleotides, particularly adenosine or guanosine nucleotides. Alternatively, substitution of pyrimidine nucleotides by modified analogues, e.g., substitution of uridine by 2′-deoxythymidine is tolerated and does not affect function of the molecule.

In one embodiment of the present invention the nucleic acid molecule may contain at least one modified nucleotide analogue. For example, the ends may be stabilized by incorporating modified nucleotide analogues.

Non-limiting examples of nucleotide analogues include sugar- and/or backbone-modified ribonucleotides (i.e., include modifications to the phosphate-sugar backbone). For example, the phosphodiester linkages of natural RNA may be modified to include at least one of a nitrogen or sulfur heteroatom. In exemplary backbone-modified ribonucleotides the phosphoester group connecting to adjacent ribonucleotides is replaced by a modified group, e.g., of phosphothioate group. In exemplary sugar-modified ribonucleotides, the 2′ OH-group is replaced by a group selected from H, OR, R, halo, SH, SR, NH₂, NHR, NR₂ or ON, wherein R is C₁-C₆ alkyl, alkenyl or alkynyl and halo is F, Cl, Br or I.

Other examples of modifications are nucleobase-modified ribonucleotides, i.e., ribonucleotides, containing at least one non-naturally occurring nucleobase instead of a naturally occurring nucleobase. Bases may be modified to block the activity of adenosine deaminase. Exemplary modified nucleobases include, but are not limited to, uridine and/or cytidine modified at the 5-position, e.g., 5-(2-amino)propyl uridine, 5-bromo uridine; adenosine and/or guanosines modified at the 8 position, e.g., 8-bromo guanosine; deaza nucleotides, e.g., 7-deaza-adenosine; 0- and N-alkylated nucleotides, e.g., N6-methyl adenosine are suitable. It should be noted that the above modifications may be combined.

In some instances, the nucleic acid molecule comprises at least one of the following chemical modifications: 2′-H, 2′-O-methyl, or 2′-OH modification of one or more nucleotides. In certain embodiments, a nucleic acid molecule of the invention can have enhanced resistance to nucleases. For increased nuclease resistance, a nucleic acid molecule, can include, for example, 2′-modified ribose units and/or phosphorothioate linkages. For example, the 2′ hydroxyl group (OH) can be modified or replaced with a number of different “oxy” or “deoxy” substituents. For increased nuclease resistance the nucleic acid molecules of the invention can include 2′-O-methyl, 2′-fluorine, 2′-O-methoxyethyl, 2′-O-aminopropyl, 2′-amino, and/or phosphorothioate linkages. Inclusion of locked nucleic acids (LNA), ethylene nucleic acids (ENA), e.g., 2′-4′-ethylene-bridged nucleic acids, and certain nucleobase modifications such as 2-amino-A, 2-thio (e.g., 2-thio-U), G-clamp modifications, can also increase binding affinity to a target.

In one embodiment, the nucleic acid molecule includes a 2′-modified nucleotide, e.g., a 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O-N-methylacetamido (2′-O-NMA). In one embodiment, the nucleic acid molecule includes at least one 2′-O-methyl-modified nucleotide, and in some embodiments, all of the nucleotides of the nucleic acid molecule include a 2′-O-methyl modification.

In certain embodiments, the nucleic acid molecule of the invention has one or more of the following properties:

Nucleic acid agents discussed herein include otherwise unmodified RNA and DNA as well as RNA and DNA that have been modified, e.g., to improve efficacy, and polymers of nucleoside surrogates. Unmodified RNA refers to a molecule in which the components of the nucleic acid, namely sugars, bases, and phosphate moieties, are the same or essentially the same as that which occur in nature, or as occur naturally in the human body. The art has referred to rare or unusual, but naturally occurring, RNAs as modified RNAs, see, e.g., Limbach et al. (Nucleic Acids Res., 1994, 22:2183-2196). Such rare or unusual RNAs, often termed modified RNAs, are typically the result of a post-transcriptional modification and are within the term unmodified RNA as used herein. Modified RNA, as used herein, refers to a molecule in which one or more of the components of the nucleic acid, namely sugars, bases, and phosphate moieties, are different from that which occur in nature, or different from that which occurs in the human body. While they are referred to as “modified RNAs” they will of course, because of the modification, include molecules that are not, strictly speaking, RNAs. Nucleoside surrogates are molecules in which the ribophosphate backbone is replaced with a non-ribophosphate construct that allows the bases to be presented in the correct spatial relationship such that hybridization is substantially similar to what is seen with a ribophosphate backbone, e.g., non-charged mimics of the ribophosphate backbone.

Modifications of the nucleic acid of the invention may be present at one or more of, a phosphate group, a sugar group, backbone, N-terminus, C-terminus, or nucleobase.

The present invention also includes a vector in which the isolated nucleic acid of the present invention is inserted. The art is replete with suitable vectors that are useful in the present invention.

In brief summary, the expression of natural or synthetic nucleic acids encoding a fusion protein of the invention is typically achieved by operably linking a nucleic acid encoding the fusion protein of the invention or portions thereof to a promoter, and incorporating the construct into an expression vector. The vectors to be used are suitable for replication and, optionally, integration in eukaryotic cells. Typical vectors contain transcription and translation terminators, initiation sequences, and promoters useful for regulation of the expression of the desired nucleic acid sequence.

The vectors of the present invention may also be used for nucleic acid immunization and gene therapy, using standard gene delivery protocols. Methods for gene delivery are known in the art. See, e.g., U.S. Pat. Nos. 5,399,346, 5,580,859, 5,589,466, incorporated by reference herein in their entireties. In another embodiment, the invention provides a gene therapy vector.

The isolated nucleic acid of the invention can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the vector may be provided to a cell in the form of a viral vector. Viral vector technology is well known in the art and is described, for example, in Sambrook et al. (2012, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

### Delivery Systems and Methods

In one aspect, the invention relates to the development of novel lentiviral packaging and delivery systems. The lentiviral particle delivers the viral enzymes as proteins. In this fashion, lentiviral enzymes are short lived, thus limiting the potential for off-target editing due to long term expression though the entire life of the cell. The incorporation of editing components, or traditional CRISPR-Cas editing components as proteins in lentiviral particles is advantageous, given that their required activity is only required for a short period of time. Thus, in one embodiment, the invention provides a lentiviral delivery system and methods of delivering the compositions of the invention, editing genetic material, and nucleic acid delivery using lentiviral delivery systems.

For example, in one aspect, the delivery system comprises (1) an packaging plasmid (2) a transfer plasmid, and (3) an envelope plasmid. In one embodiment, the packaging plasmid comprises a nucleic acid sequence encoding a modified gag-pol polyprotein. In one embodiment, the modified gag-pol polyprotein comprises integrase fused to a editing protein. In one embodiment, the modified gag-pol polyprotein comprises integrase fused to a Cas protein. In one embodiment, the modified gag-pol polyprotein comprises integrase fused to a catalytically dead Cas protein (dCas). In one embodiment, the packaging plasmid further comprises a sequence encoding a sgRNA sequence.

In one embodiment, the transfer plasmid comprises a donor sequence. The donor sequence can be any nucleic acid sequence to be delivered to a genome. In one embodiment, the transfer plasmid comprises a 5′ long terminal repeat (LTR) sequence and a 3′ LTR sequence. In one embodiment, the 3′ LTR is a Self-inactivating (SIN) LTR. Thus, in one embodiment, the 5′ LTR comprises a U3 sequence, an R sequence and a U5 sequence and the 3′ LTR comprises an R sequence and a U5 sequence, but does not comprise a U3 sequence. In one embodiment, the 5′ LTR and the 3′ LTR are specific to the Integrase in the Insctriptr packaging plasmid.

In one embodiment, the envelope plasmid comprises a nucleic acid sequence encoding an envelope protein. In one embodiment, the envelope plasmid comprises a nucleic acid sequence encoding an HIV envelope protein. In one embodiment, the envelope plasmid comprises a nucleic acid sequence encoding a vesicular stomatitis virus g-protein envelope protein. In one embodiment, the envelope protein can be selected based on the desired cell type.

In one embodiment, the packaging plasmid, transfer plasmid, and envelope plasmid are introduced into a cell. In one embodiment, the cell transcribes and translates the nucleic acid sequence encoding the modified gag-pol protein to produce the modified gag-pol protein. In one embodiment, the cell transcribes the nucleic acid sequence encoding the sgRNA. In one embodiment, the sgRNA binds to the Integrase-Cas fusion protein. In one embodiment, the cell transcribes and translates the nucleic acid sequence encoding the envelope protein to produce the envelope protein. In one embodiment, the cell transcribes the donor sequence to provide a Donor Sequence RNA molecule. In one embodiment, the modified gag-pol protein, which is bound to the sgRNA, envelope polyprotein, and donor sequence RNA are packaged into a viral particle. In one embodiment, the viral particles are collected from the cell media. In one embodiment, the viral particles transduce a target cell, wherein the sgRNA binds a target region of the cellular DNA thereby targeting the IN-Cas9 fusion protein, and the Integrase catalyzes the integration of the donor sequence into the cellular DNA.

In one aspect, the delivery system comprises (1) a packaging plasmid (2) a transfer plasmid, (3) an envelope plasmid, and (4) a VPR-IN-dCas plasmid. In one embodiment, the packaging plasmid comprises a nucleic acid sequence encoding a gag-pol polyprotein. In one embodiment, the gag-pol polyprotein comprises catalytically dead integrase. In one embodiment, the gag-pol polyprotein comprises the D116N integrase mutation.

In one embodiment, the transfer plasmid comprises a donor sequence. The donor sequence can be any nucleic acid sequence to be delivered to a genome. In one embodiment, the transfer plasmid comprises a 5′ long terminal repeat (LTR) sequence and a 3′ LTR sequence. In one embodiment, the 3′ LTR is a Self-inactivating (SIN) LTR. Thus, in one embodiment, the 5′ LTR comprises a U3 sequence, an R sequence and a U5 sequence and the 3′ LTR comprises an R sequence and a U5 sequence, but does not comprise a U3 sequence. In one embodiment, the 5′ LTR and the 3′ LTR are specific to the integrase in the VPR-IN-dCas packaging plasmid.

In one embodiment, the envelope plasmid comprises a nucleic acid sequence encoding an envelope protein. In one embodiment, the envelope plasmid comprises a nucleic acid sequence encoding an HIV envelope protein. In one embodiment, the envelope plasmid comprises a nucleic acid sequence encoding a vesicular stomatitis virus g-protein (VSV-g) envelope protein. In one embodiment, the envelope protein can be selected based on the desired cell type.

In one embodiment, the VPR-IN-dCas plasmid comprises a nucleic acid sequence encoding a fusion protein comprising VPR, integrase, and an editing protein. In one embodiment, the VPR-IN-dCas plasmid comprises a nucleic acid sequence encoding a fusion protein comprising VPR, integrase and a Cas protein. In one embodiment, the VPR-IN-dCas plasmid comprises a nucleic acid sequence encoding a fusion protein comprising VPR, integrase and a dCas protein. In one embodiment, the fusion protein comprises a protease clevage site between VPR and integrase. In one embodiment, the VPR-IN-dCas plasmid packaging plasmid further comprises a sequence encoding a sgRNA sequence.

In one embodiment, the packaging plasmid, transfer plasmid, envelope plasmid, and VPR-IN-dCas plasmid are introduced into a cell. In one embodiment, the cell transcribes and translates the nucleic acid sequence encoding the gag-pol protein to produce the gag-pol polyprotein. In one embodiment, the cell transcribes and translates the nucleic acid sequence encoding the envelope protein to produce the envelope protein. In one embodiment, the cell transcribes the donor sequence to provide a Donor Sequence RNA molecule. In one embodiment, the cell transcribes and translates the fusion protein to produce the VPR-integrase-editing protein fusion protein. In one embodiment, the cell transcribes and translates the fusion protein to produce the VPR-integrase-dCas fusion protein. In one embodiment, the cell transcribes the nucleic acid sequence encoding the sgRNA. In one embodiment, the sgRNA binds to the VPR-integrase-dCas fusion protein.

In one embodiment, the gag-pol protein, envelope polyprotein, donor sequence RNA, and VPR-integrase-dCas9 protein, which is bound to the sgRNA, are packaged into a viral particle. In one embodiment, the viral particles are collected from the cell media. In one embodiment, VPR is cleaved from the fusion protein in the viral particle via the protease site to provide a IN-dCas fusion protein. In one embodiment, the viral particles transduce a target cell, wherein the sgRNA binds a target region of the cellular DNA thereby targeting the IN-dCas fusion protein, and the integrase catalyzes the integration of the donor sequence into the cellular DNA.

In one aspect, the delivery system comprises (1) an transfer plasmid, (2) packaging plasmid, and (3) an envelope plasmid. In one embodiment, the packaging plasmid comprises a nucleic acid sequence encoding a gag-pol polyprotein. In one embodiment, the gag-pol polyprotein comprises catalytically dead integrase. In one embodiment, the gag-pol polyprotein comprises the D116N integrase mutation.

In one embodiment, the transfer plasmid comprises a nucleic acid encoding an sgRNA and a nucleic acid sequence encoding a fusion protein comprising integrase and a editing protein. In one embodiment, the transfer plasmid comprises a 5′ long terminal repeat (LTR) sequence and a 3′ LTR sequence. In one embodiment, the 3′ LTR is a Self-inactivating (SIN) LTR. Thus, in one embodiment, the 5′ LTR comprises a U3 sequence, an R sequence and a U5 sequence and the 3′ LTR comprises an R sequence and a U5 sequence, but does not comprise a U3 sequence. In one embodiment, the 5′ LTR and the 3′ LTR are specific to the integrase of the fusion protein. In one embodiment, the fusion protein comprises integrase and a Cas protein. In one embodiment, the fusion protein comprises integrase and a dCas protein. In one embodiment, the 5′LTR and 3′LTR flank the sequence encoding the fusion protein and the sequence encoding the sgRNA.

In one embodiment, the envelope plasmid comprises a nucleic acid sequence encoding an envelope protein. In one embodiment, the envelope plasmid comprises a nucleic acid sequence encoding an HIV envelope protein. In one embodiment, the envelope plasmid comprises a nucleic acid sequence encoding a vesicular stomatitis virus g-protein (VSV-g) envelope protein. In one embodiment, the envelope protein can be selected based on the desired cell type.

In one embodiment, the packaging plasmid, transfer plasmid, and envelope plasmid are introduced into a cell. In one embodiment, the cell transcribes and translates the nucleic acid sequence encoding the gag-pol protein to produce the gag-pol polyprotein. In one embodiment, the cell transcribes and translates the nucleic acid sequence encoding the envelope protein to produce the envelope protein. In one embodiment, the cell transcribes the nucleic acid sequence encoding the sgRNA. In one embodiment, the cell transcribes the nucleic acid sequence encoding the fusion protein.

In one embodiment, the gag-pol protein, envelope polyprotein, donor sequence RNA, and VPR-integrase-dCas9 protein, which is bound to the sgRNA, are packaged into a viral particle. In one embodiment, the viral particles are collected from the cell media. In one embodiment, the viral particles transduce a target cell, wherein the virus reverse translates, and the cell expresses the fusion protein and sgRNA. In one embodiment, the sgRNA binds to the Cas protein of the fusion protein and to another viral DNA transcript, wherein the integrase catalyzes self integration. In one embodiment, the sgRNA binds to the Cas protein of the fusion protein and to a target region of the cellular DNA, thereby disrupting the target gene.

In one aspect, the delivery system comprises (1) an transfer plasmid, (2) a first packaging plasmid, (3) a first envelope plasmid, (4) a second packaging plasmid, (5) a second envelope plasmid, and (6) a transfer plasmid. In one embodiment, the first packaging plasmid comprises a nucleic acid sequence encoding a gag-pol polyprotein. In one embodiment, the second packaging plasmid comprises a nucleic acid sequence encoding a gag-pol polyprotein. In one embodiment, the gag-pol polyprotein comprises catalytically dead integrase. In one embodiment, the gag-pol polyprotein comprises the D116N or D64V integrase mutation.

In one embodiment, the first envelope plasmid comprises a nucleic acid sequence encoding an envelope protein. In one embodiment, the second envelope plasmid comprises a nucleic acid sequence encoding an envelope protein. In one embodiment, the envelope plasmid comprises a nucleic acid sequence encoding an HIV envelope protein. In one embodiment, the envelope plasmid comprises a nucleic acid sequence encoding a vesicular stomatitis virus g-protein (VSV-g) envelope protein. In one embodiment, the envelope protein can be selected based on the desired cell type.

In one embodiment, the transfer plasmid comprises a nucleic acid encoding an sgRNA and a nucleic acid sequence encoding a fusion protein comprising integrase and a editing protein. In one embodiment, the fusion protein comprises integrase and a Cas protein. In one embodiment, the fusion protein comprises integrase and a dCas protein. In one embodiment, the integrase of the fusion protein is from a different species of lentivirus compared to the gag-pol polyprotein of the first and second packaging plasmid. For example, in one embodiment, the transfer plasmid comprises a nucleic acid encoding a fusion protein comprising FIV integrase and Cas, and the first and second packaging plasmids comprise a nucleic acid sequences encoding a HIV gag-pol polyprotein. In one embodiment, use of different lentiviral species prevents self-integration.

In one embodiment, the transfer plasmid comprises a 5′ long terminal repeat (LTR) sequence and a 3′ LTR sequence. In one embodiment, the 3′ LTR is a Self-inactivating (SIN) LTR. Thus, in one embodiment, the 5′ LTR comprises a U3 sequence, an R sequence and a U5 sequence and the 3′ LTR comprises an R sequence and a U5 sequence, but does not comprise a U3 sequence. In one embodiment, the 5′ LTR and the 3′ LTR are specific to the integrase of the gag-pol polyprotein. In one embodiment, the 5′LTR and 3′LTR flank the sequence encoding the fusion protein and the sequence encoding the sgRNA.

In one embodiment, the transfer plasmid comprises a donor sequence. The donor sequence can be any nucleic acid sequence to be delivered to a genome. In one embodiment, the transfer plasmid comprises a 5′ long terminal repeat (LTR) sequence and a 3′ LTR sequence. In one embodiment, the 3′ LTR is a Self-inactivating (SIN) LTR. Thus, in one embodiment, the 5′ LTR comprises a U3 sequence, an R sequence and a U5 sequence and the 3′ LTR comprises an R sequence and a U5 sequence, but does not comprise a U3 sequence. In one embodiment, the 5′ LTR and the 3′ LTR are specific to the integrase in the Inscrtipter transfer plasmid.

In one embodiment, the first packaging plasmid, transfer plasmid, and first envelope plasmid are introduced into a cell. In one embodiment, the cell transcribes and translates the nucleic acid sequence encoding the gag-pol protein to produce the gag-pol polyprotein. In one embodiment, the cell transcribes and translates the nucleic acid sequence encoding the envelope protein to produce the envelope protein. In one embodiment, the cell transcribes the nucleic acid sequence encoding the sgRNA. In one embodiment, the cell transcribes the nucleic acid sequence encoding the fusion protein. In one embodiment, the gag-pol protein, envelope polyprotein, gRNA and fusion protein RNA, are packaged into a first viral particle. In one embodiment, the first viral particles are collected from the cell media.

In one embodiment, the second packaging plasmid, transfer plasmid, and second envelope plasmid are introduced into a cell. In one embodiment, the cell transcribes and translates the nucleic acid sequence encoding the gag-pol polyprotein to produce the gag-pol polyprotein. In one embodiment, the cell transcribes and translates the nucleic acid sequence encoding the envelope protein to produce the envelope protein. In one embodiment, the cell transcribes the donor sequence to provide a Donor Sequence RNA molecule. In one embodiment, the gag-pol polyprotein, envelope polyprotein, and donor sequence RNA are packaged into a second viral particle. In one embodiment, the second viral particles are collected from the cell media.

In one embodiment, the first packaging plasmid, transfer plasmid, first envelope plasmid, the second packaging plasmid, transfer plasmid, and second envelope plasmid are introduced into the same cell. In one embodiment, the first packaging plasmid, transfer plasmid, first envelope plasmid, are introduced into a different cell as the the second packaging plasmid, transfer plasmid, and second envelope plasmid.

In one embodiment, the first viral particles and second viral particles transduce a target cell. In one embodiment, the virus reverse translates, and the cell expresses the fusion protein and sgRNA, wherein the sgRNA binds to the dCas of the fusion protein. In one embodiment, the virus reverse translates the donor sequence RNA into a donor DNA sequence, which binds to the integrase of the fusion protein. In one embodiment, the sgRNA binds a target region of the cellular DNA thereby targeting the IN-dCas fusion protein, and the integrase catalyzes the integration of the donor DNA sequence into the cellular DNA.

Further, a number of additional viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

For example, vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity.

In one embodiment, the composition includes a vector derived from an adeno-associated virus (AAV). The term “AAV vector” means a vector derived from an adeno-associated virus serotype, including without limitation, AAV-1, AAV-2, AAV-3, AAV-4, AAV-5, AAV-6, AAV-7, AAV-8, and AAV-9. AAV vectors have become powerful gene delivery tools for the treatment of various disorders. AAV vectors possess a number of features that render them ideally suited for gene therapy, including a lack of pathogenicity, minimal immunogenicity, and the ability to transduce postmitotic cells in a stable and efficient manner. Expression of a particular gene contained within an AAV vector can be specifically targeted to one or more types of cells by choosing the appropriate combination of AAV serotype, promoter, and delivery method.

AAV vectors can have one or more of the AAV wild-type genes deleted in whole or part, preferably the rep and/or cap genes, but retain functional flanking ITR sequences. Despite the high degree of homology, the different serotypes have tropisms for different tissues. The receptor for AAV1 is unknown; however, AAV1 is known to transduce skeletal and cardiac muscle more efficiently than AAV2. Since most of the studies have been done with pseudotyped vectors in which the vector DNA flanked with AAV2 ITR is packaged into capsids of alternate serotypes, it is clear that the biological differences are related to the capsid rather than to the genomes. Recent evidence indicates that DNA expression cassettes packaged in AAV 1 capsids are at least 1 log 10 more efficient at transducing cardiomyocytes than those packaged in AAV2 capsids. In one embodiment, the viral delivery system is an adeno-associated viral delivery system. The adeno-associated virus can be of serotype 1 (AAV 1), serotype 2 (AAV2), serotype 3 (AAV3), serotype 4 (AAV4), serotype 5 (AAV5), serotype 6 (AAV6), serotype 7 (AAV7), serotype 8 (AAV8), or serotype 9 (AAV9).

Desirable AAV fragments for assembly into vectors include the cap proteins, including the vp1, vp2, vp3 and hypervariable regions, the rep proteins, including rep 78, rep 68, rep 52, and rep 40, and the sequences encoding these proteins. These fragments may be readily utilized in a variety of vector systems and host cells. Such fragments may be used alone, in combination with other AAV serotype sequences or fragments, or in combination with elements from other AAV or non-AAV viral sequences. As used herein, artificial AAV serotypes include, without limitation, AAV with a non-naturally occurring capsid protein. Such an artificial capsid may be generated by any suitable technique, using a selected AAV sequence (e.g., a fragment of a vp1 capsid protein) in combination with heterologous sequences which may be obtained from a different selected AAV serotype, non-contiguous portions of the same AAV serotype, from a non-AAV viral source, or from a non-viral source. An artificial AAV serotype may be, without limitation, a chimeric AAV capsid, a recombinant AAV capsid, or a “humanized” AAV capsid. Thus exemplary AAVs, or artificial AAVs, suitable for expression of one or more proteins, include AAV2/8 (see U.S. Pat. No. 7,282,199), AAV2/5 (available from the National Institutes of Health), AAV2/9 (International Patent Publication No. WO2005/033321), AAV2/6 (U.S. Pat. No. 6,156,303), and AAVrh8 (International Patent Publication No. WO2003/042397), among others.

In certain embodiments, the vector also includes conventional control elements which are operably linked to the transgene in a manner which permits its transcription, translation and/or expression in a cell transfected with the plasmid vector or infected with the virus produced by the invention. As used herein, “operably linked” sequences include both expression control sequences that are contiguous with the gene of interest and expression control sequences that act in trans or at a distance to control the gene of interest. Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters which are native, constitutive, inducible and/or tissue-specific, are known in the art and may be utilized.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

Enhancer sequences found on a vector also regulates expression of the gene contained therein. Typically, enhancers are bound with protein factors to enhance the transcription of a gene. Enhancers may be located upstream or downstream of the gene it regulates. Enhancers may also be tissue-specific to enhance transcription in a specific cell or tissue type. In one embodiment, the vector of the present invention comprises one or more enhancers to boost transcription of the gene present within the vector.

In order to assess the expression of a fusion protein of the invention, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2012, Molecular Cloning: A Laboratory Manual, Cold Spring Harbor Laboratory, New York). An exemplary method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

### Systems

In one aspect, the present invention provides a system for editing genetic material, such as nucleic acid molecule, a genome or, a gene. In one embodiment the system comprises, in one or more vectors, a nucleic acid sequence encoding a fusion protein, wherein the fusion protein comprises a retroviral integrase (IN), or a fragment thereof; a CRISPR-associated (Cas) protein, and a nuclear localization signal (NLS); a nucleic acid sequence coding a CRISPR-Cas system guide RNA; and a nucleic acid sequence coding a donor template nucleic acid, wherein the donor template nucleic acid comprises a U3 sequence, a U5 sequence and a donor template sequence. In one embodiment, the CRISPR-Cas system guide RNA substantially hybridizes to a target DNA sequence in the gene.

In one embodiment, the system comprises, in one or more vectors, a nucleic acid sequence encoding a fusion protein, wherein the fusion protein comprises a retroviral integrase (IN), or a fragment thereof; a CRISPR-associated (Cas) protein, and a nuclear localization signal (NLS); a nucleic acid sequence coding a first CRISPR-Cas system guide RNA; a nucleic acid sequence coding a second CRISPR-Cas system guide RNA; and a nucleic acid sequence coding a donor template nucleic acid, wherein the donor template nucleic acid comprises a U3 sequence, a U5 sequence and a donor template sequence. In one embodiment, the first CRISPR-Cas system guide RNA substantially hybridizes to a first DNA sequence and the second CRISPR-Cas system guide RNA substantially hybridizes to a second DNA sequence. In one embodiment, the first DNA sequence and second DNA sequence flank a target insertion region. In one embodiment, the system catalyzes the insertion of the donor template nucleic acid into the target insertion region.

In one embodiment, the system comprises, in one or more vectors, a nucleic acid sequence encoding a first fusion protein, wherein the first fusion protein comprises a retroviral integrase (IN), or a fragment thereof, a CRISPR-associated (Cas) protein, and a nuclear localization signal (NLS); a nucleic acid sequence coding a first CRISPR-Cas system guide RNA; a nucleic acid sequence encoding a second fusion protein, wherein the second fusion protein comprises a retroviral integrase (IN), or a fragment thereof, a CRISPR-associated (Cas) protein, and a nuclear localization signal (NLS); a nucleic acid sequence coding a first CRISPR-Cas system guide RNA; a nucleic acid sequence coding a second CRISPR-Cas system guide RNA; and a nucleic acid sequence coding a donor template nucleic acid, wherein the donor template nucleic acid comprises a U3 sequence, a U5 sequence and a donor template sequence.

In one embodiment, the first fusion protein and the second fusion protein are the same or are different. For example, in one embodiment, the first fusion protein comprises a HIV IN, or a fragment thereof, a dCas9 protein, and a NLS; and the second fusion protein comprises a BIV IN, or a fragment thereof, a Cpf1 Cas protein, and a NLS.

In one embodiment the U3 is specific to the retroviral IN of the first fusion protein and the U5 is specific to the retroviral IN of the second fusion protein. For example, in one embodiment, the first fusion protein comprises a HIV IN, or a fragment thereof, a dCas9 protein, and a NLS; the second fusion protein comprises a BIV IN, or a fragment thereof, a Cpf1 Cas protein, and a NLS; the U3 sequence is specific to HIV IN and the U5 sequence is specific to BIV IN.

In one embodiment, the first CRISPR-Cas system guide RNA substantially hybridizes to a first DNA sequence and the second CRISPR-Cas system guide RNA substantially hybridizes to a second DNA sequence. In one embodiment, the first DNA sequence and second DNA sequence flank a target insertion region. In one embodiment, the system catalyzes the insertion of the donor template nucleic acid into the target insertion region.

In one embodiment the system comprises a nucleic acid sequence encoding a fusion protein, wherein the fusion protein comprises a retroviral integrase (IN), or a fragment thereof; a CRISPR-associated (Cas) protein, and a nuclear localization signal (NLS); a CRISPR-Cas system guide RNA; a donor template nucleic acid, wherein the donor template nucleic acid comprises a U3 sequence, a U5 sequence and a donor template sequence.

In one embodiment, the nucleic acid sequence encoding a fusion protein, nucleic acid sequence coding a CRISPR-Cas system guide RNA, and the nucleic acid sequence coding a donor template nucleic acid are on the same or different vectors.

In one embodiment, the nucleic acid sequence encoding a fusion protein encodes a fusion protein comprising a sequence at least 70%, at least 71%, at least 72%, at least 73%, at least 74%, at least 75%, at least 76%, at least 77%, at least 78%, at least 79%, 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% identical to one of SEQ ID NOs:57-98. In one embodiment, the nucleic acid sequence encoding a fusion protein encodes a fusion protein comprising a sequence of one of SEQ ID NOs:57-98.

In one embodiment, the nucleic acid sequence encoding a fusion protein comprises a nucleic acid sequence at least 70%, at least 71%, at least 72%, at least 73%, at least 74%, at least 75%, at least 76%, at least 77%, at least 78%, at least 79%, 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% identical to one of SEQ ID NOs:155-196. In one embodiment, the nucleic acid sequence encoding a fusion protein comprises a nucleic acid sequence of one of SEQ ID NOs:155-196.

In one embodiment, the U3 sequence and U5 sequence are specific to the retroviral IN. For example, in one embodiment, the retroviral IN is HIV IN and the U3 sequence comprises a sequence at least 70%, at least 71%, at least 72%, at least 73%, at least 74%, at least 75%, at least 76%, at least 77%, at least 78%, at least 79%, 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% identical to SEQ ID NO:197 and the U5 sequence comprises a sequence at least 70%, at least 71%, at least 72%, at least 73%, at least 74%, at least 75%, at least 76%, at least 77%, at least 78%, at least 79%, 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% identical to SEQ ID NO:198.

In one embodiment, the retroviral IN is RSV IN and the U3 sequence comprises a sequence at least 70%, at least 71%, at least 72%, at least 73%, at least 74%, at least 75%, at least 76%, at least 77%, at least 78%, at least 79%, 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% identical to SEQ ID NO:199 and the U5 sequence comprises a sequence 95% identical to SEQ ID NO:200.

In one embodiment, the retroviral IN is HFV IN and the U3 sequence comprises a sequence at least 70%, at least 71%, at least 72%, at least 73%, at least 74%, at least 75%, at least 76%, at least 77%, at least 78%, at least 79%, 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% identical to SEQ ID NO:201 and the U5 sequence comprises a sequence at least 70%, at least 71%, at least 72%, at least 73%, at least 74%, at least 75%, at least 76%, at least 77%, at least 78%, at least 79%, 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% identical to SEQ ID NO:202.

In one embodiment, the retroviral IN is EIAV IN and the U3 sequence comprises a sequence at least 70%, at least 71%, at least 72%, at least 73%, at least 74%, at least 75%, at least 76%, at least 77%, at least 78%, at least 79%, 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% identical to SEQ ID NO:203 and the U5 sequence comprises a sequence at least 70%, at least 71%, at least 72%, at least 73%, at least 74%, at least 75%, at least 76%, at least 77%, at least 78%, at least 79%, 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% identical to SEQ ID NO:204.

In one embodiment, the retroviral IN is MoLV IN and the U3 sequence comprises a sequence at least 70%, at least 71%, at least 72%, at least 73%, at least 74%, at least 75%, at least 76%, at least 77%, at least 78%, at least 79%, 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% identical to SEQ ID NO:205 and the U5 sequence comprises a sequence at least 70%, at least 71%, at least 72%, at least 73%, at least 74%, at least 75%, at least 76%, at least 77%, at least 78%, at least 79%, 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% identical to SEQ ID NO:206.

In one embodiment, the retroviral IN is MMTV IN and the U3 sequence comprises a sequence at least 70%, at least 71%, at least 72%, at least 73%, at least 74%, at least 75%, at least 76%, at least 77%, at least 78%, at least 79%, 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% identical to SEQ ID NO:207 and the U5 sequence comprises a sequence at least 70%, at least 71%, at least 72%, at least 73%, at least 74%, at least 75%, at least 76%, at least 77%, at least 78%, at least 79%, 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% identical to SEQ ID NO:208.

In one embodiment, the retroviral IN is WDSV IN and the U3 sequence comprises a sequence at least 70%, at least 71%, at least 72%, at least 73%, at least 74%, at least 75%, at least 76%, at least 77%, at least 78%, at least 79%, 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% identical to SEQ ID NO:209 and the U5 sequence comprises a sequence at least 70%, at least 71%, at least 72%, at least 73%, at least 74%, at least 75%, at least 76%, at least 77%, at least 78%, at least 79%, 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% identical to SEQ ID NO:210.

In one embodiment, the retroviral IN is BLV IN and the U3 sequence comprises a sequence at least 70%, at least 71%, at least 72%, at least 73%, at least 74%, at least 75%, at least 76%, at least 77%, at least 78%, at least 79%, 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% identical to SEQ ID NO:211 and the U5 sequence comprises a sequence at least 70%, at least 71%, at least 72%, at least 73%, at least 74%, at least 75%, at least 76%, at least 77%, at least 78%, at least 79%, 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% identical to SEQ ID NO:212.

In one embodiment, the retroviral IN is SIV IN and the U3 sequence comprises a sequence at least 70%, at least 71%, at least 72%, at least 73%, at least 74%, at least 75%, at least 76%, at least 77%, at least 78%, at least 79%, 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% identical to SEQ ID NO:213 and the U5 sequence comprises a sequence at least 70%, at least 71%, at least 72%, at least 73%, at least 74%, at least 75%, at least 76%, at least 77%, at least 78%, at least 79%, 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% identical to SEQ ID NO:214.

In one embodiment, the retroviral IN is FIV IN and the U3 sequence comprises a sequence at least 70%, at least 71%, at least 72%, at least 73%, at least 74%, at least 75%, at least 76%, at least 77%, at least 78%, at least 79%, 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% identical to SEQ ID NO:215 and the U5 sequence comprises a 70%, at least 71%, at least 72%, at least 73%, at least 74%, at least 75%, at least 76%, at least 77%, at least 78%, at least 79%, 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% identical identical to SEQ ID NO:216.

In one embodiment, the retroviral IN is BIV IN and the U3 sequence comprises a sequence at least 70%, at least 71%, at least 72%, at least 73%, at least 74%, at least 75%, at least 76%, at least 77%, at least 78%, at least 79%, 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% identical to SEQ ID NO:217 and the U5 sequence comprises a sequence at least 70%, at least 71%, at least 72%, at least 73%, at least 74%, at least 75%, at least 76%, at least 77%, at least 78%, at least 79%, 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% identical to SEQ ID NO:218.

In one embodiment, the IN is TY1 and the U3 sequence comprises a sequence at least 70%, at least 71%, at least 72%, at least 73%, at least 74%, at least 75%, at least 76%, at least 77%, at least 78%, at least 79%, 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% identical to SEQ ID NO:219 and the U5 sequence comprises a sequence at least 70%, at least 71%, at least 72%, at least 73%, at least 74%, at least 75%, at least 76%, at least 77%, at least 78%, at least 79%, 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% identical to SEQ ID NO:220.

In one embodiment, the IN is InsF IN and the U3 sequence is a IS3 IRL sequence and the U5 sequence is a IS3 IRR sequence. In one embodiment, the IN is InsF IN and the U3 sequence comprises a sequence at least 70%, at least 71%, at least 72%, at least 73%, at least 74%, at least 75%, at least 76%, at least 77%, at least 78%, at least 79%, 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% identical to SEQ ID NO:221 and the U5 sequence comprises a sequence at least 70%, at least 71%, at least 72%, at least 73%, at least 74%, at least 75%, at least 76%, at least 77%, at least 78%, at least 79%, 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% identical to SEQ ID NO:222.

The systems and vectors can be designed for expression of CRISPR transcripts (e.g. nucleic acid transcripts, proteins, or enzymes) in prokaryotic or eukaryotic cells. For example, CRISPR transcripts can be expressed in bacterial cells such as Escherichia coli, insect cells (using baculovirus expression vectors), yeast cells, or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector systems can be transcribed and translated in vitro, for example using T7 promoter regulatory sequences and T7 polymerase.

Vectors may be introduced and propagated in a prokaryote. In some embodiments, a prokaryote is used to amplify copies of a vector to be introduced into a eukaryotic cell or as an intermediate vector in the production of a vector to be introduced into a eukaryotic cell (e.g. amplifying a plasmid as part of a viral vector packaging system). In some embodiments, a prokaryote is used to amplify copies of a vector and express one or more nucleic acids, such as to provide a source of one or more proteins for delivery to a host cell or host organism. Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, such as to the amino terminus of the recombinant protein. Such fusion vectors may serve one or more purposes, such as: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Example fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A. respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

In some embodiments, a vector is a yeast expression vector. Examples of vectors for expression in yeast Saccharomyces cerivisae include pYepSec1 (Baldari, et al., 1987. EMBO J. 6: 229-234), pMFa (Kuijan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

In some embodiments, a vector drives protein expression in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In some embodiments, a vector is capable of driving expression of one or more sequences in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In some embodiments, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Baneiji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the a-fetoprotein promoter (Campes and Tilghman, 1989. Genes Dev. 3: 537-546).

In some embodiments, a regulatory element is operably linked to one or more elements of a CRISPR system so as to drive expression of the one or more elements of the CRISPR system. In general, CRISPRs (Clustered Regularly Interspaced Short Palindromic Repeats), also known as SPIDRs (SPacer Interspersed Direct Repeats), constitute a family of DNA loci that are usually specific to a particular bacterial species. The CRISPR locus comprises a distinct class of interspersed short sequence repeats (SSRs) that were recognized in E. coli (Ishino et al., J. Bacteriol., 169:5429-5433 [1987]; and Nakata et al., J. Bacteriol., 171:3553-3556 [1989]), and associated genes. Similar interspersed SSRs have been identified in Haloferax mediterranei, Streptococcus pyogenes, Anabaena, and Mycobacterium tuberculosis (See, Groenen et al., Mol. Microbiol., 10:1057-1065 [1993]; Hoe et al., Emerg. Infect. Dis., 5:254-263 [1999]; Masepohl et al., Biochim. Biophys. Acta 1307:26-30 [1996]; and Mojica et al., Mol. Microbiol., 17:85-93 [1995]). The CRISPR loci typically differ from other SSRs by the structure of the repeats, which have been termed short regularly spaced repeats (SRSRs) (Janssen et al., OMICS J. Integ. Biol., 6:23-33 [2002]; and Mojica et al., Mol. Microbiol., 36:244-246 [2000]). In general, the repeats are short elements that occur in clusters that are regularly spaced by unique intervening sequences with a substantially constant length (Mojica et al., [2000], supra). Although the repeat sequences are highly conserved between strains, the number of interspersed repeats and the sequences of the spacer regions typically differ from strain to strain (van Embden et al., J. Bacteriol., 182:2393-2401 [2000]). CRISPR loci have been identified in more than 40 prokaryotes (See e.g., Jansen et al., Mol. Microbiol., 43:1565-1575 [2002]; and Mojica et al., [2005]) including, but not limited to Aeropyrum, Pyrobaculum, Sulfolobus, Archaeoglobus, Halocarcula, Methanobacteriumn, Methanococcus, Methanosarcina, Methanopyrus, Pyrococcus, Picrophilus, Thernioplasnia, Corynebacterium, Mycobacterium, Streptomyces, Aquifrx, Porphvromonas, Chlorobium, Thermus, Bacillus, Listeria, Staphylococcus, Clostridium, Thermoanaerobacter, Mycoplasma, Fusobacterium, Azarcus, Chromobacterium, Neisseria, Nitrosomonas, Desulfovibrio, Geobacter, Myrococcus, Campylobacter, Wolinella, Acinetobacter, Erwinia, Escherichia, Legionella, Methylococcus, Pasteurella, Photobacterium, Salmonella, Xanthomonas, Yersinia, Treponema, and Thermotoga.

As used herein, a “target sequence” refers to a sequence to which a guide sequence is designed to have complementarity, where hybridization between a target sequence and a guide sequence promotes the formation of a CRISPR complex. Full complementarity is not necessarily required, provided there is sufficient complementarity to cause hybridization and promote formation of a CRISPR complex. A target sequence may comprise any polynucleotide, such as DNA or RNA polynucleotides. In some embodiments, a target sequence is located in the nucleus or cytoplasm of a cell. In some embodiments, the target sequence may be within an organelle of a eukaryotic cell, for example, mitochondrion or chloroplast. A sequence or template that may be used for recombination into the targeted locus comprising the target sequences is referred to as an “editing template” or “editing polynucleotide” or “editing sequence”. In aspects of the invention, an exogenous template polynucleotide may be referred to as an editing template. In an aspect of the invention the recombination is homologous recombination.

A guide sequence may be selected to target any target sequence. In some embodiments, the target sequence is a sequence within a genome of a cell. Exemplary target sequences include those that are unique in the target genome. For example, for the S. pyogenes Cas9, a unique target sequence in a genome may include a Cas9 target site of the form MMMMMMMMNNNNNNNNNNNNXGG where NNNNNNNNNNNNXGG (N is A, G, T, or C; and X can be anything) has a single occurrence in the genome. A unique target sequence in a genome may include an S. pyogenes Cas9 target site of the form MMMMMMMMMNNNNNNNNNNNXGG where NNNNNNNNNNNXGG (N is A, G, T, or C; and X can be anything) has a single occurrence in the genome. For the S. thermophilus CRISPR1 Cas9, a unique target sequence in a genome may include a Cas9 target site of the form MMMMMMMMNNNNNNNNNNNNXXAGAAW (SEQ ID NO: 1) where NNNNNNNNNNNNXXAGAAW (SEQ ID NO: 2) (N is A, G, T, or C; X can be anything; and W is A or T) has a single occurrence in the genome. A unique target sequence in a genome may include an S. thermophilus CRISPR1 Cas9 target site of the form MMMMMMMMMNNNNNNNNNNNXXAGAAW (SEQ ID NO: 3) where NNNNNNNNNNNXXAGAAW (SEQ ID NO: 4) (N is A, G, T, or C; X can be anything; and W is A or T) has a single occurrence in the genome. For the S. pyogenes Cas9, a unique target sequence in a genome may include a Cas9 target site of the form MMMMMMMMNNNNNNNNNNNNXGGXG where NNNNNNNNNNNNXGGXG (N is A, G, T, or C; and X can be anything) has a single occurrence in the genome. A unique target sequence in a genome may include an S. pyogenes Cas9 target site of the form MMMMMMMMMNNNNNNNNNNNXGGXG where NNNNNNNNNNNXGGXG (N is A, G, T, or C; and X can be anything) has a single occurrence in the genome. In each of these sequences “M” may be A, G, T, or C, and need not be considered in identifying a sequence as unique.

In some embodiments, a guide sequence is selected to reduce the degree of secondary structure within the guide sequence. Secondary structure may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g. A. R. Gruber et al., 2008, Cell 106(1): 23-24; and P A Carr and GM Church, 2009, Nature Biotechnology 27(12): 1151-62).

In general, a tracr mate sequence includes any sequence that has sufficient complementarity with a tracr sequence to promote one or more of: (1) excision of a guide sequence flanked by tracr mate sequences in a cell containing the corresponding tracr sequence; and (2) formation of a CRISPR complex at a target sequence, wherein the CRISPR complex comprises the tracr mate sequence hybridized to the tracr sequence. In general, degree of complementarity is with reference to the optimal alignment of the tracr mate sequence and tracr sequence, along the length of the shorter of the two sequences. Optimal alignment may be determined by any suitable alignment algorithm, and may further account for secondary structures, such as self-complementarity within either the tracr sequence or tracr mate sequence. In some embodiments, the degree of complementarity between the tracr sequence and tracr mate sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and tracr mate sequence are contained within a single transcript, such that hybridization between the two produces a transcript having a secondary structure, such as a hairpin. In one embodiment, loop forming sequences for use in hairpin structures are four nucleotides in length. In one embodiment, loop forming sequences for use in hairpin structures have the sequence GAAA. However, longer or shorter loop sequences may be used, as may alternative sequences. The sequences may include a nucleotide triplet (for example, AAA), and an additional nucleotide (for example C or G). Examples of loop forming sequences include CAAA and AAAG. In an embodiment of the invention, the transcript or transcribed polynucleotide sequence has at least two or more hairpins. In some embodiments, the transcript has two, three, four or five hairpins. In a further embodiment of the invention, the transcript has at most five hairpins. In some embodiments, the single transcript further includes a transcription termination sequence; in some embodiments this is a polyT sequence, for example six T nucleotides.

### Methods of Editing and Delivery Nucleic Acids

In one embodiment, the present invention provides methods of editing genetic material, such as nucleic acid molecule, a genome or, a gene. For example, in one embodiment, editing is integration. In one embodiment, editing is CIRSPR-mediated editing.

In one embodiment, the method comprises administering to the genetic material: a nucleic acid molecule encoding a fusion protein; a guide nucleic acid comprising a targeting nucleotide sequence complimentary to a target region in the genetic material; and a donor template nucleic acid comprising a U3 sequence, a U5 sequence and a donor template sequence. In one embodiment, the method comprises administering to the genetic material: a fusion protein; a guide nucleic acid comprising a targeting nucleotide sequence complimentary to a target region in the genetic material; and a donor template nucleic acid comprising a U3 sequence, a U5 sequence and a donor template sequence. In one embodiment, the method is and in vitro method or an in vivo method.

In one embodiment, the present invention provides methods of delivering a nucleic acid sequence to genetic material. In one embodiment, the method comprises administering to the gene: a nucleic acid molecule encoding a fusion protein; a guide nucleic acid comprising a targeting nucleotide sequence complimentary to a target region in the gene; and a donor template nucleic acid comprising a U3 sequence, a U5 sequence and a donor template sequence. In one embodiment, the method comprises administering to the genetic material: a fusion protein; a guide nucleic acid comprising a targeting nucleotide sequence complimentary to a target region in the genetic material; and a donor template nucleic acid comprising a U3 sequence, a U5 sequence and a donor template sequence. In one embodiment, the method is and in vitro method or an in vivo method.

In one embodiment, the method comprises administering to a cell a nucleic acid molecule encoding a fusion protein; a guide nucleic acid comprising a targeting nucleotide sequence complimentary to a target region in the gene; and a donor template nucleic acid comprising a U3 sequence, a U5 sequence and a donor template sequence. In one embodiment, the method comprises administering to a cell a fusion protein; a guide nucleic acid comprising a targeting nucleotide sequence complimentary to a target region in the gene; and a donor template nucleic acid comprising a U3 sequence, a U5 sequence and a donor template sequence.

In one embodiment, the method of editing genetic material is a method of editing a gene. In one embodiment, the gene is located in the genome of the cell. In one embodiment, the method of editing genetic material is a method of editing a nucleic acid.

In one embodiment, the invention provides methods of inserting a donor template sequence into a target sequence. In one embodiment, the method inserts a donor template sequence into a target sequence in a cell. In one embodiment, the method comprises administering to the cell a nucleic acid molecule encoding a fusion protein; a guide nucleic acid comprising a targeting nucleotide sequence complimentary to a region in the target sequence; and a donor template nucleic acid comprising a U3 sequence, a U5 sequence and the donor template sequence. In one embodiment, the method comprises administering to the cell a fusion protein; a guide nucleic acid comprising a targeting nucleotide sequence complimentary to a region in the target sequence; and a donor template nucleic acid comprising a U3 sequence, a U5 sequence and the donor template sequence.

Targeted delivery of large DNA sequences for genome editing using CRISPR-Cas9 mediated HDR remains inefficient. However, the present invention provides methods for inserting a large donor template sequence into a target sequence in a cell. In one embodiment the method inserts donor template sequence at least 1 kb or more, at least 2 kb or more, at least 3 kb or more, at least 4 kb or more, at least 5 kb or more, at least 6 kb or more, at least 7 kb or more, at least 8 kb or more, at least 9 kb or more, at least 10 kb or more, at least 11 kb or more, at least 12 kb or more, at least 13 kb or more, at least 14 kb or more, at least 15 kb or more, at least 16 kb or more, at least 17 kb or more, or at least 18 kb or more. In one embodiment, the method comprises administering to the cell a fusion protein or a nucleic acid molecule encoding a fusion protein; a guide nucleic acid comprising a targeting nucleotide sequence complimentary to a region in the target sequence; and a donor template nucleic acid comprising a U3 sequence, a U5 sequence and the donor template sequence.

In one embodiment, the target sequence is located within a gene. In one embodiment, the donor template sequence disrupts the sequence of a gene thereby inhibiting or reducing the expression of the gene. In one embodiment, target sequence has a mutation and the donor template sequence inserts a corrected sequence into the target sequence, thereby correcting the gene mutation. In one embodiment, the donor template sequence is a gene sequence and inserting the donor template sequence into a target sequence in a cell allows for expression of the gene.

In one embodiment, the donor template sequence is inserted into a safe harbor site. Thus, in one embodiment, the guide nucleic acid comprising a nucleotide sequence complimentary to a safe harbor region in the gene. Safe harbor regions allow for expression of a therapeutic gene without affecting neighbor gene expression. Safe harbor regions may include intergenic regions apart from neighbor genes ex. H11, or within ‘non-essential’ genes, ex. CCR5, hROSA26 or AAVS1. Exemplary safe harbor regions and guide nucleic acid sequences complementary to these sequences can be found, for example in Pellenz et al., New Human Chromosomal Sites with “Safe Harbor” Potential for Targeted Transgene Insertion, 2019, Hum Gene Ther 30(7):814-28, which is herein incorporated by reference.

In one embodiment, the donor template sequence is inserted into a 3′ untranslated region (UTR) allowing the expression of the donor template sequence to be controlled by the the promoters of other genes.

In one embodiment, the nucleic acid molecule comprises a first nucleic acid sequence encoding a retroviral integrase (IN), or a fragment thereof; a second nucleic acid sequence encoding a CRISPR-associated (Cas) protein; and a third nucleic acid sequence encoding a nuclear localization signal (NLS).

In one embodiment, the retroviral IN is human immunodeficiency virus (HIV) IN, Rous sarcoma virus (RSV) IN, Mouse mammary tumor virus (MMTV) IN, Moloney murine leukemia virus (MoLV) IN, bovine leukemia virus (BLV) IN, Human T-lymphotropic virus (HTLV) IN, avian sarcoma leukosis virus (ASLV) IN, feline leukemia virus (FLV) IN, xenotropic murine leukemia virus-related virus (XMLV) IN, simian immunodeficiency virus (SIV) IN, feline immunodeficiency virus (FIV) IN, equine infectious anemia virus (EIAV) IN, Prototype foamy virus (PFV) IN, simian foamy virus (SFV) IN, human foamy virus (HFV) IN, walleye dermal sarcoma virus (WDSV) IN, or bovine immunodeficiency virus (BIV) IN.

In one embodiment, the retroviral IN is HIV IN. In one embodiment, the HIV IN comprises one or more amino acid substitutions, wherein the substitution improves catalytic activity, improves solubility, or increases interaction with one or more host cellular cofactors. In one embodiment, HIV IN comprises one or more amino acid substitutions selected from the group consisting of E85G, E85F, D116N, F185K, C280S, T97A, Y134R, G140S, and Q148H. In one embodiment, HIV IN comprises amino acid substitutions F185K and C280S. In one embodiment, HIV IN comprises amino acid substitutions T97A and Y134R. In one embodiment, HIV IN comprises amino acid substitutions G140S and Q148H.

In one embodiment, the retroviral IN fragment comprises the IN N-terminal domain (NTD), and the IN catalytic core domain (CCD). In one embodiment, the retroviral IN fragment comprises the IN CCD and the IN C-terminal domain (CTD). In one embodiment, the retroviral IN fragment comprises the IN NTD. In one embodiment, the retroviral IN fragment comprises the IN CCD. In one embodiment, the retroviral IN fragment comprises the IN CTD.

In one embodiment, the first nucleic acid sequence encoding a retroviral IN comprises a nucleic acid sequence encoding a sequence at least 95% identical to one of SEQ ID NOs:1-40. In one embodiment, the first nucleic acid sequence encoding a retroviral IN comprises a nucleic acid sequence encoding a sequence at least 96% identical to one of SEQ ID NOs:1-40. In one embodiment, the first nucleic acid sequence encoding a retroviral IN comprises a nucleic acid sequence encoding a sequence at least 97% identical to one of SEQ ID NOs:1-40. In one embodiment, the first nucleic acid sequence encoding a retroviral IN comprises a nucleic acid sequence encoding a sequence at least 98% identical to one of SEQ ID NOs:1-40. In one embodiment, the first nucleic acid sequence encoding a retroviral IN comprises a nucleic acid sequence encoding a sequence at least 99% identical to one of SEQ ID NOs:1-40. In one embodiment, the first nucleic acid sequence encoding a retroviral IN comprises a nucleic acid sequence encoding one of SEQ ID NOs:1-40.

In one embodiment, the first nucleic acid sequence encoding a retroviral IN comprises a nucleic acid sequence at least at least 95% identical to one of SEQ ID NOs:99-138. In one embodiment, the first nucleic acid sequence encoding a retroviral IN comprises a nucleic acid sequence at least at least 96% identical to one of SEQ ID NOs:99-138. In one embodiment, the first nucleic acid sequence encoding a retroviral IN comprises a nucleic acid sequence at least at least 97% identical to one of SEQ ID NOs:99-138. In one embodiment, the first nucleic acid sequence encoding a retroviral IN comprises a nucleic acid sequence at least at least 98% identical to one of SEQ ID NOs:99-138. In one embodiment, the first nucleic acid sequence encoding a retroviral IN comprises a nucleic acid sequence at least at least 99% identical to one of SEQ ID NOs:99-138. In one embodiment, the first nucleic acid sequence encoding a retroviral IN comprises a nucleic acid sequence of one of SEQ ID NOs:99-138.

In one embodiment, the Cas protein is Cas9, Cas13, or Cpf1. In one embodiment, the Cas protein is catalytically deficient (dCas).

In one embodiment, the second nucleic acid sequence encoding a Cas protein comprises a nucleic acid sequence encoding a sequence at least 95% identical to one of SEQ ID NOs:41-46. In one embodiment, the second nucleic acid sequence encoding a Cas protein comprises a nucleic acid sequence encoding a sequence at least 96% identical to one of SEQ ID NOs:41-46. In one embodiment, the second nucleic acid sequence encoding a Cas protein comprises a nucleic acid sequence encoding a sequence at least 97% identical to one of SEQ ID NOs:41-46. In one embodiment, the second nucleic acid sequence encoding a Cas protein comprises a nucleic acid sequence encoding a sequence at least 98% identical to one of SEQ ID NOs:41-46. In one embodiment, the second nucleic acid sequence encoding a Cas protein comprises a nucleic acid sequence encoding a sequence at least 99% identical to one of SEQ ID NOs:41-46. In one embodiment, the second nucleic acid sequence encoding a Cas protein comprises a nucleic acid sequence encoding one of SEQ ID NOs:41-46.

In one embodiment, the second nucleic acid sequence encoding a Cas protein comprises a nucleic acid sequence at least at least 95% identical to one of SEQ ID NOs:139-144. In one embodiment, the second nucleic acid sequence encoding a Cas protein comprises a nucleic acid sequence at least at least 96% identical to one of SEQ ID NOs:139-144. In one embodiment, the second nucleic acid sequence encoding a Cas protein comprises a nucleic acid sequence at least at least 97% identical to one of SEQ ID NOs:139-144. In one embodiment, the second nucleic acid sequence encoding a Cas protein comprises a nucleic acid sequence at least at least 98% identical to one of SEQ ID NOs:139-144. In one embodiment, the second nucleic acid sequence encoding a Cas protein comprises a nucleic acid sequence at least at least 99% identical to one of SEQ ID NOs:139-144. In one embodiment, the second nucleic acid sequence encoding a Cas protein comprises a nucleic acid sequence of one of SEQ ID NOs:139-144.

In one embodiment, the NLS is a retrotransposon NLS. In one embodiment, the NLS is derived from yeast GAL4, SKI3, L29 or histone H2B proteins, polyoma virus large T protein, VP1 or VP2 capsid protein, SV40 VP1 or VP2 capsid protein, Adenovirus El a or DBP protein, influenza virus NS1 protein, hepatitis vims core antigen or the mammalian lamin, c-myc, max, c-myb, p53, c-erbA, jun, Tax, steroid receptor or Mx proteins, or simian vims 40 (“SV40”) T-antigen. In one embodiment, the NLS is a Ty1 or Ty1-derived NLS, a Ty2 or Ty2-derived NLS or a MAK11 or MAK11-derived NLS. In one embodiment, the Ty1 NLS comprises an amino acid sequence of SEQ ID NO:51. In one embodiment, the Ty2 NLS comprises an amino acid sequence of SEQ ID NO:254. In one embodiment, the MAK11 NLS comprises an amino acid sequence of SEQ ID NO:256.

In one embodiment, third nucleic acid sequence encoding a NLS comprises a nucleic acid sequence encoding a sequence at least 95% identical to one of SEQ ID NOs:47-56. In one embodiment, third nucleic acid sequence encoding a NLS comprises a nucleic acid sequence encoding a sequence at least 96% identical to one of SEQ ID NOs:47-56. In one embodiment, the third nucleic acid sequence encoding a NLS comprises a nucleic acid sequence encoding a sequence at least 97% identical to one of SEQ ID NOs:47-56. In one embodiment, third nucleic acid sequence encoding a NLS comprises a nucleic acid sequence encoding a sequence at least 98% identical to one of SEQ ID NOs:47-56. In one embodiment, third nucleic acid sequence encoding a NLS comprises a nucleic acid sequence encoding a sequence at least 99% identical to one of SEQ ID NOs:47-56. In one embodiment, third nucleic acid sequence encoding a NLS comprises a nucleic acid sequence encoding one of SEQ ID NOs:47-56.

In one embodiment, third nucleic acid sequence encoding a NLS comprises a nucleic acid sequence at least at least 95% identical to one of SEQ ID NOs:145-154. In one embodiment, third nucleic acid sequence encoding a NLS comprises a nucleic acid sequence at least at least 96% identical to one of SEQ ID NOs:145-154. In one embodiment, third nucleic acid sequence encoding a NLS comprises a nucleic acid sequence at least at least 97% identical to one of SEQ ID NOs:145-154. In one embodiment, third nucleic acid sequence encoding a NLS comprises a nucleic acid sequence at least at least 98% identical to one of SEQ ID NOs:145-154. In one embodiment, third nucleic acid sequence encoding a NLS comprises a nucleic acid sequence at least at least 99% identical to one of SEQ ID NOs:145-154. In one embodiment, third nucleic acid sequence encoding a NLS comprises a nucleic acid sequence of one of SEQ ID NOs:145-154.

In one embodiment, the nucleic acid molecule encodes a fusion protein comprising a sequence at least 95% identical to one of SEQ ID NOs:57-98. In one embodiment, the nucleic acid molecule encodes a fusion protein comprising a sequence at least 96% identical to one of SEQ ID NOs:57-98. In one embodiment, the nucleic acid molecule encodes a fusion protein comprising a sequence at least 97% identical to one of SEQ ID NOs:57-98. In one embodiment, the nucleic acid molecule encodes a fusion protein comprising a sequence at least 98% identical to one of SEQ ID NOs:57-98. In one embodiment, the nucleic acid molecule encodes a fusion protein comprising a sequence at least 99% identical to one of SEQ ID NOs:57-98. In one embodiment, the nucleic acid molecule encodes a fusion protein comprising a sequence of one of SEQ ID NOs:57-98.

In one embodiment, the nucleic acid molecule comprises a nucleic acid sequence at least 95% identical to one of SEQ ID NOs:155-196. In one embodiment, the nucleic acid molecule comprises a nucleic acid sequence at least 96% identical to one of SEQ ID NOs:155-196. In one embodiment, the nucleic acid molecule comprises a nucleic acid sequence at least 97% identical to one of SEQ ID NOs:155-196. In one embodiment, the nucleic acid molecule comprises a nucleic acid sequence at least 98% identical to one of SEQ ID NOs:155-196. In one embodiment, the nucleic acid molecule comprises a nucleic acid sequence at least 99% identical to one of SEQ ID NOs:155-196. In one embodiment, the nucleic acid molecule comprises a nucleic acid sequence of one of SEQ ID NOs:155-196.

In one embodiment, the U3 sequence and U5 sequence are specific to the retroviral IN

In some embodiments, the gene is any target gene of interest. For example in one embodiment, the gene is any gene associated an increase in the risk of having or developing a disease. In some embodiments, the method comprises introducing the nucleic acid molecule encoding a fusion protein; the guide nucleic acid comprising a targeting nucleotide sequence complimentary to a target region in the gene; and the donor template nucleic acid comprising a U3 sequence, a U5 sequence and a donor template sequence. In one embodiment, the IN-Cas9 fusion protein binds to a target polynucleotide to effect cleavage of the target polynucleotide within the gene. In one embodiment, the IN-Cas9 fusion protein is complexed with the guide nucleic acid that is hybridized to the target sequence within the target polynucleotide. In one embodiment, the IN-Cas9 fusion protein is complexed with the nucleic acid sequence coding a donor template nucleic acid. In one embodiment, the IN-Cas9 fusion protein is complexed with the nucleic acid sequence coding a guide nucleic acid. In one embodiment, the IN-Cas9 fusion protein is complexed with the nucleic acid sequence coding a guide nucleic acid and the nucleic acid sequence coding a donor template nucleic acid. In one embodiment, the IN-Cas9 fusion protein is complexed with the guide nucleic acid that is hybridized to the target sequence within the target polynucleotide and the donor template nucleic acid. In one embodiment, the IN-Cas9 fusion protein is complexed with the donor template nucleic acid. In one embodiment, the IN-Cas9 fusion protein is complexed with the guide nucleic acid. In one embodiment, the IN-Cas9 fusion protein is complexed with the guide nucleic acid and the donor template nucleic acid.

In some embodiments, the IN-Cas9 catalyzes the integration of the donor template into to the gene. In one embodiment, the integration introduces one or more mutations into the gene. In some embodiments, said mutation results in one or more amino acid changes in a protein expression from a gene comprising the target sequence.

In one embodiment, the IN-mediated integration of DNA sequences can occur in either direction in a target DNA sequence. In one embodiment, different combinations of Cas and IN retroviral class proteins are used to promote direction editing. For example, in one embodiment, a fusion of IN from a retroviral class is bound to a first catalytically dead Cas allowing for binding to a specific target sequence utilizing the Cas-specific guide-RNA. In one embodiment, the donor sequence comprises both HIV and BIV LTR sequences. Thus, in one embodiment, the sequence is integrated in a single orientation with the target DNA.

In one embodiment, flanking LoxP (Floxed) sequences are incorporated around a gene of interest. Including floxed sequences allows for CRE-mediated recombination and conditional mutagenesis. Current methods to generate Floxed alleles using CRISPR-Cas9 are inefficient. The most widely utilized approach is to use two guide-RNAs to induce DNA cleavage at flanking target sequences and Homology Direct Repair to insert ssDNA templates containing LoxP sequences. However, when using double sgRNAs to induce cleavage, the most favorable reaction is the deletion of intervening sequence, resulting in global gene deletion. Thus, in one embodiment, the use of Integrase-Cas-mediated gene insertion increases the efficiency of tandem insertion of DNA sequences. In one embodiment, the integration of a sequence containing inverted LoxP sequences allows for recombination of flanking LoxP sequences because IN-mediated integration may occur in either the direction.

### Methods of Treatment and Use

The present invention provides methods of treating, reducing the symptoms of, and/or reducing the risk of developing a disease or disorder and/or genetic modification to produce a desired phenotypic outcome. For example, in one embodiment, methods of the invention of treat, reduce the symptoms of, and/or reduce the risk of developing a disease or disorder in a mammal. In one embodiment, the methods of the invention of treat, reduce the symptoms of, and/or reduce the risk of developing a disease or disorder in a plant. In one embodiment, the methods of the invention of treat, reduce the symptoms of, and/or reduce the risk of developing a disease or disorder in a yeast organism.

In one embodiment, the disease or disorder is caused by one or more mutations in a genomic locus. Thus, in one embodiment, the disease or disorder is may be treated, reduced, or the risk can be reduced via introducing a nucleic acid sequence that corresponds to the wild type sequence of the region having the one or more mutations and/or introducing an element that prevents or reduces the expression of the genomic sequence having the one or more mutations. Thus, in one embodiment, the method comprises manipulation of a target sequence within a coding, non-coding or regulatory element of the genomic locus in a target sequence.

For example, in one embodiment, the disease is a monogenic disease. In one embodiment, the disease includes, but is not limited to, Duchenne muscular dystrophy (mutations occurring in Dystrophin), Limb-Girdle Muscular Dystrophy type 2B (LGMD2B) and Miyoshi myopathy (mutations occurring in Dysferlin), Cystic Fibrosis (mutations occurring in CFTR), Wilson's disease (mutations occurring in ATP7B) and Stargardt Macular Degeneration (mutations occurring in ABCA4).

The present invention also provides methods of modulating the expression of a gene or genetic material. For example, in one embodiment, the methods of the invention provide deliver a genetic material to confer a phenotype in a cell or organism. For example, in one embodiment, the method provides resistance to pathogens. In one embodiment, the method provides for modulation of metabolic pathways. In one embodiment, the method provides for the production and use of a material in an organism. For example, in one embodiment, the method generates a material, such as a biologic, a pharmaceutical, and a biofuel, in an organism such as a eukaryote, yeast, bacteria, or plant.

In one embodiment, the method comprises administering a fusion protein or a nucleic acid molecule encoding a fusion protein; a guide nucleic acid comprising a targeting nucleotide sequence complimentary to a target region in the gene; and a donor template nucleic acid comprising a U3 sequence, a U5 sequence. In one embodiment, the method further comprises administering a donor template sequence.

In one embodiment, the target sequence is located within a gene. In one embodiment, the donor template sequence disrupts the sequence of a gene thereby inhibiting or reducing the expression of the gene. In one embodiment, target sequence has a mutation and the donor template sequence inserts a corrected sequence into the target sequence, thereby correcting the gene mutation. In one embodiment, the donor template sequence is a gene sequence and inserting the donor template sequence into a target sequence in a cell allows for expression of the gene.

In one embodiment, the fusion protein comprises a CRISPR-associated (Cas) protein and a nuclear localization signal (NLS). In one embodiment, the fusion protein comprises a Cas protein, a NLS and a retroviral integrase (IN), or a fragment thereof.

In one embodiment, the retroviral IN is human immunodeficiency virus (HIV) IN, Rous sarcoma virus (RSV) IN, Mouse mammary tumor virus (MMTV) IN, Moloney murine leukemia virus (MoLV) IN, bovine leukemia virus (BLV) IN, Human T-lymphotropic virus (HTLV) IN, avian sarcoma leukosis virus (ASLV) IN, feline leukemia virus (FLV) IN, xenotropic murine leukemia virus-related virus (XMLV) IN, simian immunodeficiency virus (SIV) IN, feline immunodeficiency virus (FIV) IN, equine infectious anemia virus (EIAV) IN, Prototype foamy virus (PFV) IN, simian foamy virus (SFV) IN, human foamy virus (HFV) IN, walleye dermal sarcoma virus (WDSV) IN, or bovine immunodeficiency virus (BIV) IN.

In one embodiment, the retroviral IN is HIV IN. In one embodiment, the HIV IN comprises one or more amino acid substitutions, wherein the substitution improves catalytic activity, improves solubility, or increases interaction with one or more host cellular cofactors. In one embodiment, HIV IN comprises one or more amino acid substitutions selected from the group consisting of E85G, E85F, D116N, F185K, C280S, T97A, Y134R, G140S, and Q148H. In one embodiment, HIV IN comprises amino acid substitutions F185K and C280S. In one embodiment, HIV IN comprises amino acid substitutions T97A and Y134R. In one embodiment, HIV IN comprises amino acid substitutions G140S and Q148H.

In one embodiment, the retroviral IN fragment comprises the IN N-terminal domain (NTD), and the IN catalytic core domain (CCD). In one embodiment, the retroviral IN fragment comprises the IN CCD and the IN C-terminal domain (CTD). In one embodiment, the retroviral IN fragment comprises the IN NTD. In one embodiment, the retroviral IN fragment comprises the IN CCD. In one embodiment, the retroviral IN fragment comprises the IN CTD.

In one embodiment, the retroviral IN comprises a sequence at least 70%, at least 71%, at least 72%, at least 73%, at least 74%, at least 75%, at least 76%, at least 77%, at least 78%, at least 79%, 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% identical to one of SEQ ID NOs:1-40. In one embodiment, the retroviral IN comprises a sequence of one of SEQ ID NOs:1-40.

In one embodiment, the nucleic acid encoding the retroviral IN comprises a nucleic acid sequence at least at least 70%, at least 71%, at least 72%, at least 73%, at least 74%, at least 75%, at least 76%, at least 77%, at least 78%, at least 79%, 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% identical SEQ ID NOs:99-138. In one embodiment, the nucleic acid encoding the encoding a retroviral IN comprises a nucleic acid sequence of one of SEQ ID NOs:99-138.

In one embodiment, the Cas protein is Cas9, Cas13, or Cpf1. In one embodiment, the Cas protein is catalytically deficient (dCas).

In one embodiment, the Cas protein comprises sequence sequence at least 70%, at least 71%, at least 72%, at least 73%, at least 74%, at least 75%, at least 76%, at least 77%, at least 78%, at least 79%, 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% identical to one of SEQ ID NOs:41-46. In one embodiment, the Cas protein comprises a sequence of one of SEQ ID NOs:41-46.

In one embodiment, the nucleic acid sequence encoding a Cas protein comprises a nucleic acid sequence at least 70%, at least 71%, at least 72%, at least 73%, at least 74%, at least 75%, at least 76%, at least 77%, at least 78%, at least 79%, 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% identical to one of SEQ ID NOs:139-144. In one embodiment, the nucleic acid sequence encoding a Cas protein comprises a nucleic acid sequence of one of SEQ ID NOs:139-144.

In one embodiment, the NLS is a retrotransposon NLS. In one embodiment, the NLS is derived from yeast GAL4, SKI3, L29 or histone H2B proteins, polyoma virus large T protein, VP1 or VP2 capsid protein, SV40 VP1 or VP2 capsid protein, Adenovirus El a or DBP protein, influenza virus NS1 protein, hepatitis vims core antigen or the mammalian lamin, c-myc, max, c-myb, p53, c-erbA, jun, Tax, steroid receptor or Mx proteins, or simian vims 40 (“SV40”) T-antigen. In one embodiment, the NLS is a Ty1 or Ty1-derived NLS, a Ty2 or Ty2-derived NLS or a MAK11 or MAK11-derived NLS. In one embodiment, the Ty1 NLS comprises an amino acid sequence of SEQ ID NO:51. In one embodiment, the Ty2 NLS comprises an amino acid sequence of SEQ ID NO:254. In one embodiment, the MAK11 NLS comprises an amino acid sequence of SEQ ID NO:256.

In one embodiment, NLS comprises a nucleic acid sequence encoding a sequence at least 70%, at least 71%, at least 72%, at least 73%, at least 74%, at least 75%, at least 76%, at least 77%, at least 78%, at least 79%, 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% identical to one of SEQ ID NOs:47-56, 254-256 and 275-887. In one embodiment, NLS comprises a nucleic acid sequence encoding one of SEQ ID NOs: 47-56, 254-256 and 275-887.

In one embodiment, the nucleic acid sequence encoding a NLS comprises a nucleic acid sequence at least 70%, at least 71%, at least 72%, at least 73%, at least 74%, at least 75%, at least 76%, at least 77%, at least 78%, at least 79%, 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% identical to one of SEQ ID NOs:145-154. In one embodiment, nucleic acid sequence encoding a NLS comprises a nucleic acid sequence of one of SEQ ID NOs:145-154.

In one embodiment, the fusion protein comprises a sequence at least 70%, at least 71%, at least 72%, at least 73%, at least 74%, at least 75%, at least 76%, at least 77%, at least 78%, at least 79%, 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% identical to one of SEQ ID NOs:57-98. In one embodiment, the fusion protein comprises a sequence of one of SEQ ID NOs:57-98.

In one embodiment, the U3 sequence and U5 sequence are specific to the retroviral IN

In some embodiments, the gene is any target gene of interest. For example, in one embodiment, the gene is any gene associated an increase in the risk of having or developing a disease. In some embodiments, the method comprises introducing the nucleic acid molecule encoding a fusion protein; the guide nucleic acid comprising a targeting nucleotide sequence complimentary to a target region in the gene; and the donor template nucleic acid comprising a U3 sequence, a U5 sequence and a donor template sequence. In one embodiment, the IN-Cas9 fusion protein binds to a target polynucleotide to effect cleavage of the target polynucleotide within the gene. In one embodiment, the IN-Cas9 fusion protein is complexed with the guide nucleic acid that is hybridized to the target sequence within the target polynucleotide. In one embodiment, the IN-Cas9 fusion protein is complexed with the nucleic acid sequence coding a donor template nucleic acid. In one embodiment, the IN-Cas9 fusion protein is complexed with the nucleic acid sequence coding a guide nucleic acid. In one embodiment, the IN-Cas9 fusion protein is complexed with the nucleic acid sequence coding a guide nucleic acid and the nucleic acid sequence coding a donor template nucleic acid. In one embodiment, the IN-Cas9 fusion protein is complexed with the guide nucleic acid that is hybridized to the target sequence within the target polynucleotide and the donor template nucleic acid. In one embodiment, the IN-Cas9 fusion protein is complexed with the donor template nucleic acid. In one embodiment, the IN-Cas9 fusion protein is complexed with the guide nucleic acid. In one embodiment, the IN-Cas9 fusion protein is complexed with the guide nucleic acid and the donor template nucleic acid.

In some embodiments, the IN-Cas9 catalyzes the integration of the donor template into to the gene. In one embodiment, the integration introduces one or more mutations into the gene. In some embodiments, said mutation results in one or more amino acid changes in a protein expression from a gene comprising the target sequence.

### EXPERIMENTAL EXAMPLES

The invention is further described in detail by reference to the following experimental examples. These examples are provided for purposes of illustration only and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out certain embodiments of the present invention, and are not to be construed as limiting in any way the remainder of the disclosure.

### Example 1: Enhanced Nuclear Localization of Retroviral Integrase-dCas9 Fusion Proteins for Editing of Mammalian Genomic DNA

Efficient CRISPR-Cas9 editing of mammalian genomic DNA requires the nuclear localization of Cas9, a large, bacterial RNA-guided endonuclease that normally functions in prokaryotic cells lacking nuclear membranes. Efficient nuclear localization of Cas9 in mammalian cells has been shown to require the addition of at least two mammalian nuclear localization signals, one located at the N-terminus and one at the C-terminus (Cong et al., 2013, Science 339:819-23).

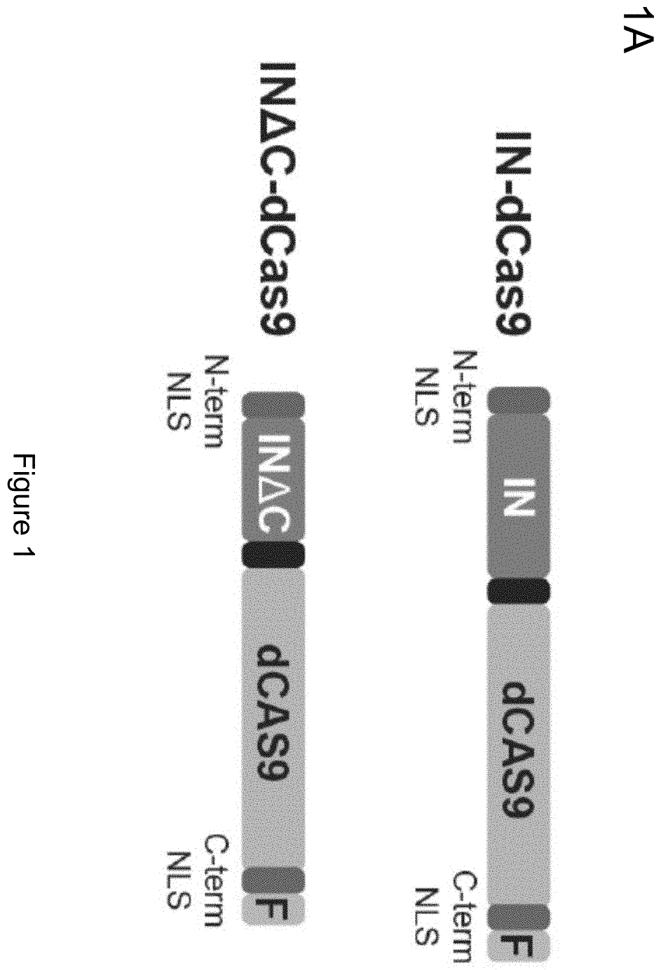

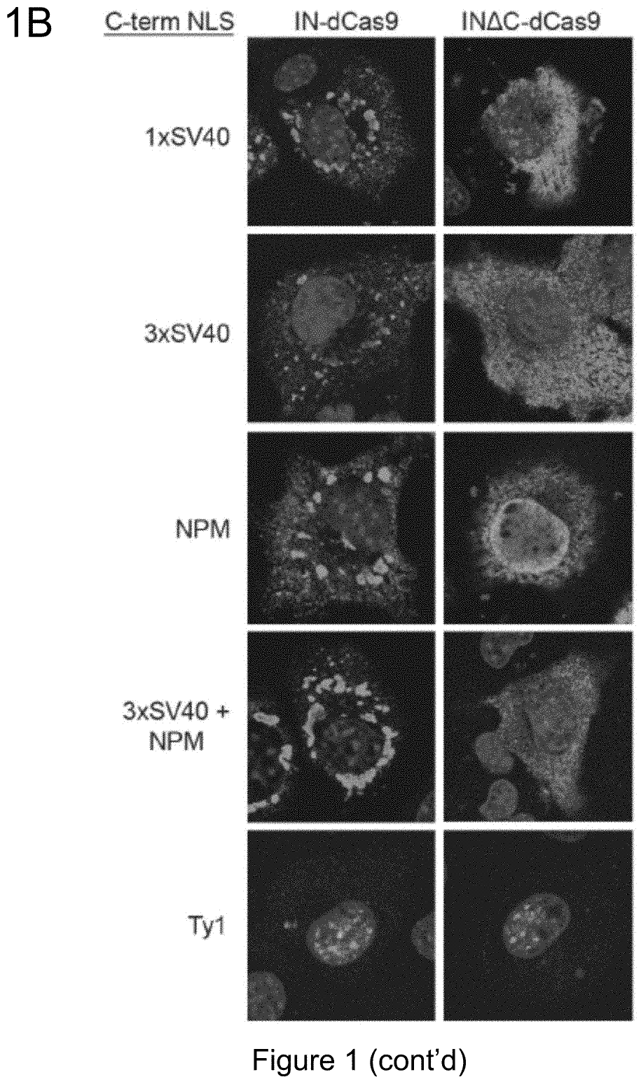

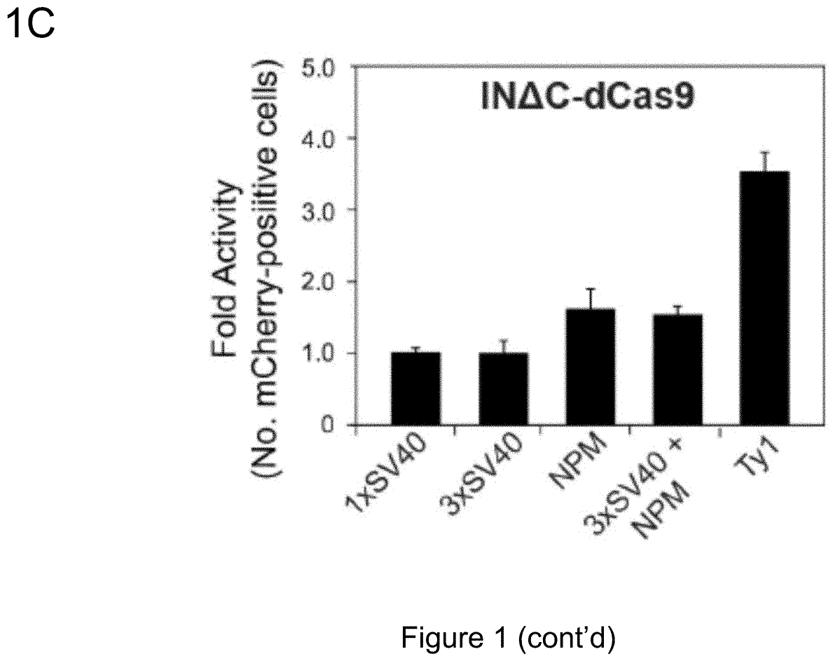

To promote nuclear localization of the retroviral Integrase-dCas9 fusion proteins for editing, an N-terminal SV40 NLS was included on Integrase, in addition to a C-terminal SV40 NLS on dCas9 (FIG. 1A). Surprisingly, when expressed in mammalian cells, only a small fraction of the IN-dCas9 fusion proteins were nuclear localized, as detected using a FLAG antibody recognizing the C-terminal 3×FLAG epitope on dCas9 (FIG. 1B). Interestingly while the full-length IN-dCas9 fusion protein gave rise to cytoplasmic aggregates, deletion of the C-terminal domain of Integrase (INΔC) resulted in greater solubility and increased nuclear localization (FIG. 1B).

The fusion of retroviral Integrase to dCas9 appears to dramatically decrease its ability to localize to the nucleus. To further enhance the nuclear localization of IntegrasedCas9 fusion proteins, a number of different mammalian nuclear localization sequences were tested for their ability to direct IN-dCas9 nuclear import (FIG. 1B). Multimerizing 3 copies of the SV40 NLS (3×SV40) had no apparent effect on the degree of nuclear localization of IN-dCas9 or INΔC-dCas9. However, the addition of the bipartite NLS from Nucleoplasmin (NPM) provided increased nuclear localization of the INΔC-dCas9 fusion protein, but not that of the full-length IN fusion protein. The combination of the 3×SV40 and NPM NLS appeared similar to NPM alone.

Interestingly, yeast LTR-retrotransposons (for example Ty1) are the evolutionary ancestors of retroviruses and replicate their genomes through reverse transcription of an RNA intermediate in the cytoplasm (Curcio et al., 2015, Microbiol Spectr 3:MDNA3-0053-2014). LTR-retrotransposons contain an integrase enzyme, which is required for the insertion of the retrotransposon genome. As opposed to higher eukaryotes which undergo open mitosis during cell division, yeast undergo closed mitosis, whereby their nuclear envelope remains intact. Thus, for Ty1 biogenesis, nuclear import of the integrase/retrotransposon genome complex requires active nuclear import. Thus, in contrast to mammalian Integrase enzymes, the Ty1 integrase contains a large C-terminal bipartite NLS which is required for retrotransposition (Moore et al., 1998, Mol Cell Biol 18:1105-14). Interestingly, the results presented herein demonstrate that fusion of the Ty1 NLS to the C-terminus of both IN-dCas9 fusion proteins provided robust nuclear localization in mammalian cells (FIG. 1B).

The increased nuclear localization of INΔC-dCas9 fusion protein significantly enhanced editing in dividing mammalian cells in culture. The addition of the Ty1 NLS enhanced the activity of INΔC-dCas9 fusion protein to integrate an IRES-mCherry template targeted to the 3′UTRE of EF1-alpha in HEK293 cells (FIG. 1C). Utilizing the robust Ty1 NLS may further allow for editing in non-dividing cells, which always maintain a nuclear envelope (for example, in vivo therapeutic applications).

### Example 2: An Integrated Gene Editing Approach for the Correction of Muscular Dystrophy

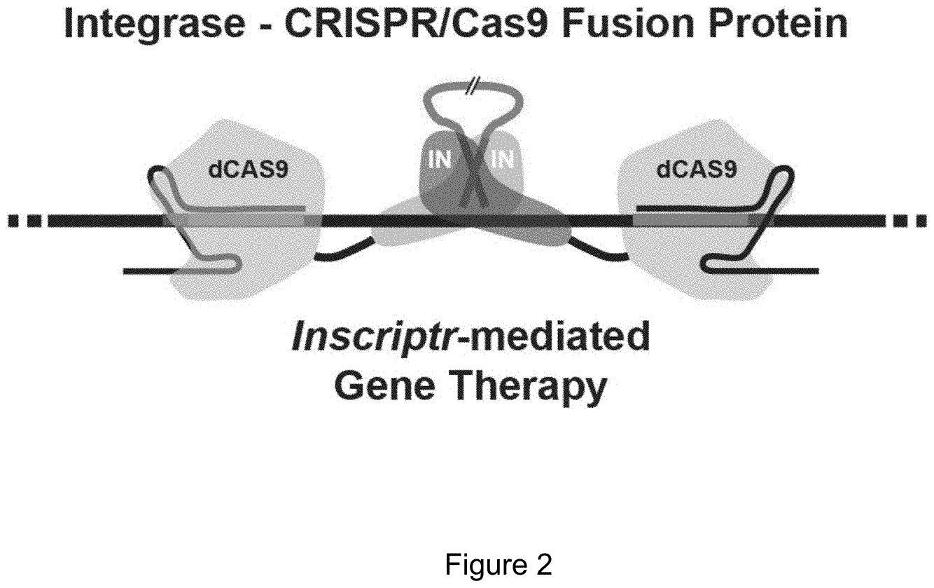

As demonstrated elsewhere herein, fusion of lentiviral Integrase to CRISPR-Cas9 allows for the sequence-specific integration of large DNA sequences into genomic DNA. This approach can be utilized for the delivery of therapeutically beneficial genes to non-pathogenic genomic locations (safe harbors) for the permanent correction of human genetic diseases (FIG. 2). This technology allows for the sequence-specific integration of large DNA donor sequences containing short viral end motifs.

The major advantage of the gene therapy approach of the invention is the ability to deliver donor DNA sequences to targeted genome locations. Further, this approach eliminates the need for homology arms and relies on targeting by guide-RNAs, greatly simplifying genome editing. Thus, once a specific reporter donor sequence is generated, it can be guided to any location (or multiple locations) for diverse applications.

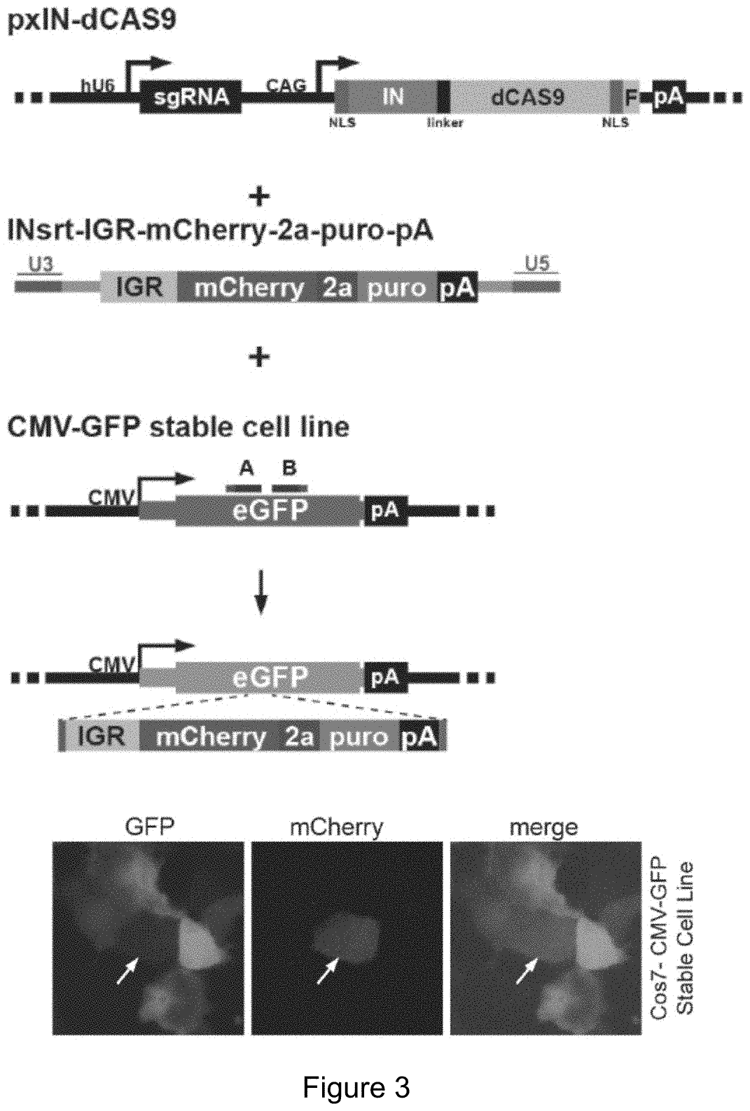

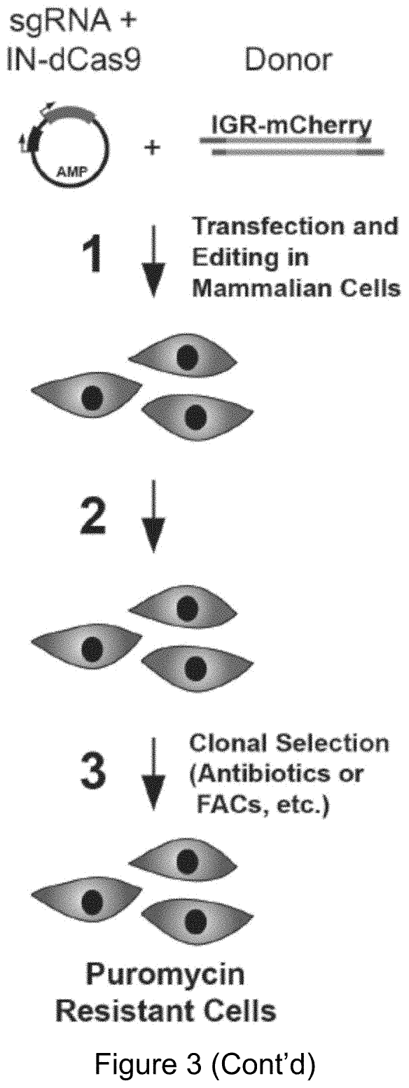

Fusion of lentiviral Integrase to dCas9 is sufficient to insert donor DNA sequences containing short viral termini to target sequences using CRISPR guide-RNAs in mammalian cells (FIG. 3). To monitor Integrase-Cas-mediated integration in mammalian cells, donor vector containing the IGR IRES sequence followed by an mCherry-2a-puromycin gene and an SV40 polyadenylation sequence were generated (FIG. 3). Next, sgRNAs targeting a stable human CMV-eGFP stable cell line in COS-7 cells were designed. The hCMV-eGFP stable transgene provided a heterologous target sequence which can be used to determine editing at a robustly expressed but non-essential expression locus. Donor mCherry-2a-puro templates were purified and co-transfected with sgRNAs and IN-dCas9 into the GFP stable cells and cultured for 48 hours. After 48 hours, mCherry-positive cells were visible in culture and replaced the GFP positive signal (FIG. 3).

Efficacy and Fidelity of Integrase-Cas-Mediated Integration of Human Dystrophin into Mammalian Genomes.

Integrase-Cas-mediated gene delivery directs the sequence-specific integration of large DNA sequences into mammalian genomic DNA. Integrase-Cas is used to deliver the human Dystrophin gene under the control of the Human α-Skeletal Actin (HSA) promoter to safe harbor locations using CRISPR guide-RNAs specific to human AAVS1 and mouse ROSA26 genomic DNA in cultured cells. Correct targeting of Dystrophin is assessed using PCR-based genotyping.

Integrase-Cas-Mediated Dystrophin Gene Therapy Restores Muscle Function in a Mouse Model of Duchenne Muscular Dystrophy.

The efficacy of Inscritpr-mediated delivery of human Dystrophin is determined in the MDX mouse line, the most commonly used mouse model for muscular dystrophy. Following systemic delivery, the levels of dystrophin expression are quantified and measured in limb skeletal muscle, heart and diaphragm using an anti-dystrophin antibody over a time-course of 2, 4 and 6 months. Mitigation of DMD disease pathogenesis is assessed by quantifying the levels of serum Creatine Kinase (CK) (a marker of skeletal muscle damage and diagnostic marker for DMD patients), grip strength and histological analyses of limb skeletal muscle, heart and diaphragm.

Histological Analyses of Gene Expression.

At 8 weeks of age, left hindlimb quadriceps muscle, heart, and diaphragm are harvested, weighed and fixed in 4% formaldehyde in PBS and processed using routine methods for paraffin histology. The percentage of myofibers expressing the HSA-dystrophin/GFP fusion protein is performed using an anti-GFP antibody in both DMD^{Mdx/y} and WT mice. The right hindlimb muscles are flash frozen in liquid nitrogen for subsequent PCR-based genotyping, gene expression by RT-PCR and protein expression analyses by western blot.

Integrase-Cas-mediated delivery mitigates disease pathogenesis in a mouse model of Duschenne muscular dystrophy.

Haematoxylin and eosin (H&E), von Kossa and Masson's trichrome staining of transverse histological sections is used to identify myofibers containing centralized nuclei, mineralization and endomysial fibrosis, respectively. Quantitative comparisons and statistical analyses are used to compare the ratio of myofibers with centralized nuclei or compare the area of mineralization or fibrosis that is stained in quadriceps limb muscle. At least three different sectional planes are compared for each muscle, from 3 different mice of each genotype. Integrase-Cas treated Dmd^{mdx/y} which mice show a less severe phenotype, have decreased ratio of myfibers with centralized nuclei and less total area of fibrosis and mineralization.

Serum Creatine Kinase (CK) Measurements.

Serum CK is a correlated marker of skeletal muscle damage and diagnostic marker for DMD patients. CK measurements are performed at 2, 4, 6, and 8 weeks on the above cohort of animals using non-lethal procedures. Briefly, blood is harvested from the periorbital vascular plexus directly into microhematocrit tubes, allowed to clot at room temperature for 30 minutes and then centrifuged at 1,700×g for 10 minutes. Treated mice showing a less severe phenotype than Dmd^{mdx/y} KO, have significantly decreased serum CK levels,

### Example 3: Genome Editing—Directed Non-Homologous DNA Integration

The data presented herein demonstrates optimized Integrase-Cas to enable efficient editing of mammalian genomes.

Optimized Editing

To optimize IN-mediated integration, it is determined whether amino acid mutations that enhance Integrase catalytic activity, solubility, or interaction with host cellular cofactors enhance editing. Further, the efficiency and fidelity of IN proteins isolated from the seven unique classes of retrovirus are evaluated.

To quantify and characterize IN-dCas9 mediated integration in mammalian cells, a plasmid-based reporter system is used that utilizes the blue chromoprotein from the coral Acropora millepora (amilCP), which produces dark blue colonies when expressed in Escherichia coli. Disruption of the amilCP open reading frame abolishes blue protein expression, which can be used as a direct readout for targeting fidelity. Further, a donor template encoding the chloramphenicol antibiotic resistance gene, flanked by the U3 and U5 retroviral end sequences from HIV was generated. Integration of this donor template confers resistance to chloramphenicol, which can be utilized to monitor Integrase-Cas-mediated DNA integration. In this reporter assay, expression plasmids containing the IN-dCas9 fusion protein, sgRNAs targeting amilCP and donor template are co-transfected into mammalian COS-7 cells with the bacterial amilCP reporter. After 48 hours, total plasmid DNA is recovered using column purification and transformed into E. coli. IN-dCas9 is sufficient to integrate the chloramphenicol encoding template DNA into the amilCP reporter plasmid, thereby disrupting amilCP expression and conferring resistance to chloramphenicol. This rapid assay, which allows for quantification and clonal sequence analysis of individual integration events, is used for optimizing editing.

Enhancing Integrase Activity: While most mutations within IN abolish its activity, decades of past research have identified a few mutations which enhance IN integration by increasing IN catalytic activity (D116N), dimerization (E85F), solubility (F185K/C280S) and interaction with host cellular proteins (K71R). IN-dCas9 fusion proteins containing activating IN mutations are used to determine if this enhances activity using the plasmid-based reporter assay.

Modification of Integrase activity by host cellular proteins: While IN is the only protein necessary and sufficient to integrate proviral DNA in vitro, interactions with host cellular proteins can greatly alter IN-mediated DNA integration18. Notably, LEDGF/p75, VBP1, and SNF5 are a well-characterized HIV IN interacting proteins which can promote IN-mediated integration. These factors are expressed using the plasmid reporter assay to determine if they enhance donor template integration.

Compare and contrast Integrases from different retroviral classes: While all IN enzymes from retroviral classes contain the conserved core catalytic D,D(35)E residues, they differ greatly in genome size, complexity, U3 and U5 terminal sequences and DNA joining efficiencies. To determine the editing efficiencies of different retroviral INs, model examples from each retroviral class are cloned as a fusion to dCas9, including Alpha (RSV), Beta (MMTV), Gamma (MoLV), Delta (BLV), Epsilon (WDSV) and Spumavirus (HFV). Donor plasmids are generated containing their respective U3 and U5 terminal motifs. Protein expression is verified by western blot and nuclear localization is verified using immunocytochemistry using a FLAG antibody to detect the 3×FLAG epitope located on the C-terminus of dCas9.

Efficiency of Editing of Mammalian Genomic DNA

The efficacy and fidelity of editing of mammalian genomic DNA is determined using a stable CMV-driven GFP reporter cell-line and generate a donor template containing an RFP and puromycin selection cassette. Integration events are quantified and clonally characterized to determine the efficacy and fidelity of the method as a novel genome editing technology.

Generation of a cell-based reporter assay: To quantify integration events at this locus, a donor template is used containing an IRES-RFP-2A-puromycin cassette and guide-RNAs targeting the GFP coding sequence. Upon insertion of the donor cassette into the CMV-GFP locus, RFP expression replaces GFP expression and provides resistance to the antibiotic puromycin. The efficiency and fidelity of Inscripr editing is quantified using FACS sorting to determine the percentage of cells that are RFP+/GFP− (targeted integration) after transfection and 48 hours of culture. Puromycin is used to select for clonal integration events, which is characterized using PCR primers to amplify the sequences between the GFP locus and the donor cassette.

Editing at multiple endogenous loci: Integrase-Cas is used to knock-in the RFP-2Apuromycin cassette using sgRNAs specific to the CMV-GFP locus and to the 3′UTR of the human EF1-alpha locus in the HEK293 human cell line. Targeting the 3′UTR allows for expression of the IRES-dependent vector, while not disrupting normal gene expression. After clonal selection using puromycin, PCR-genotyping is used to determine the percentage of clones that have integrated the donor template at both loci.

### Example 4: Generation and Characterization of Incriptr

### Generation of a Functional IN-dCas9 Fusion Protein.

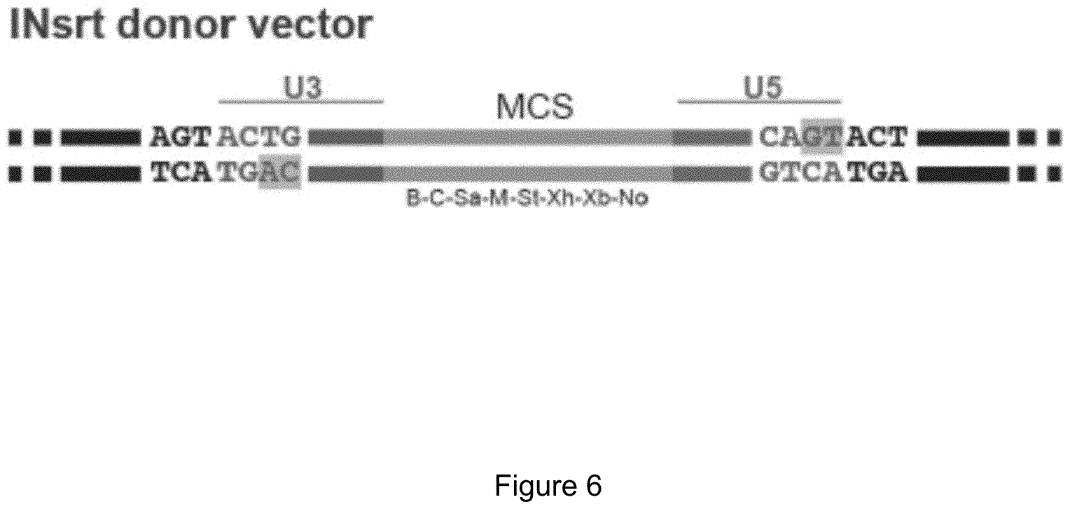

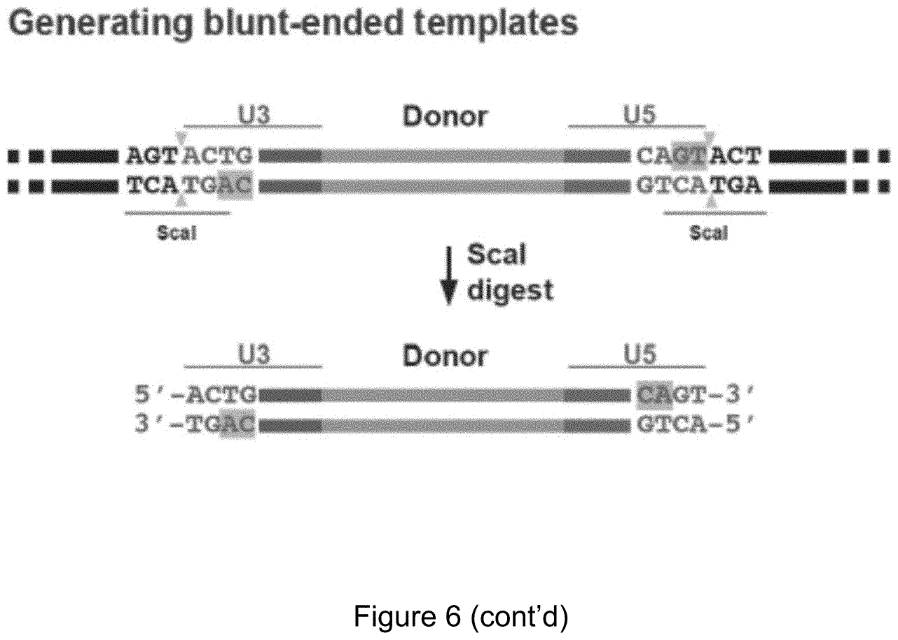

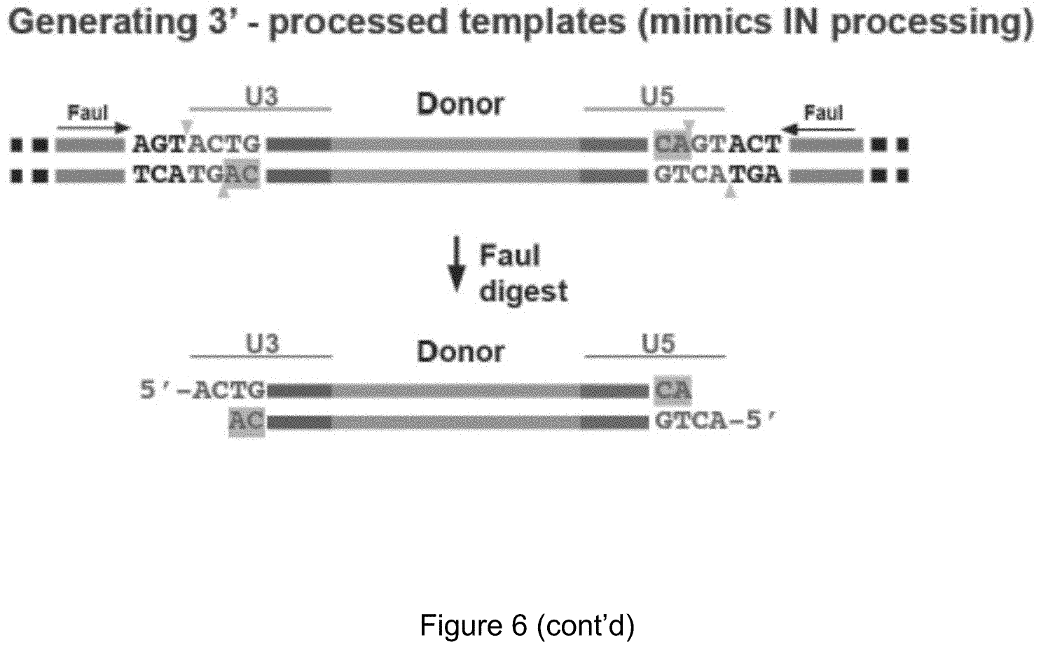

To generate a functional IN-dCas9 fusion protein for use in mammalian cells, full-length retroviral IN was cloned from HIV-1 (amino acids 1148-1435 of the gag-pol polyprotein), separated by a flexible 15 amino acid linker [(GGGGS)3)] to the N-terminus of human codon-optimized dCas9 (FIG. 6). An SV40 nuclear localization signal (NLS) was included at the N-terminus of IN, which together with the C-terminal SV40 NLS on dCas9, provided nuclear localization of the IN-dCas9 fusion protein. To generate an IN-dCas9 fusion lacking the C-terminal non-specific DNA binding domain, an additional construct was generated containing only the N-terminal and catalytic core domains of IN (a.a. 1148-1369) as an N-terminal fusion to dCas9 (FIG. 6).

### Generation of a Reporter for Monitoring Editing of Plasmid DNA.

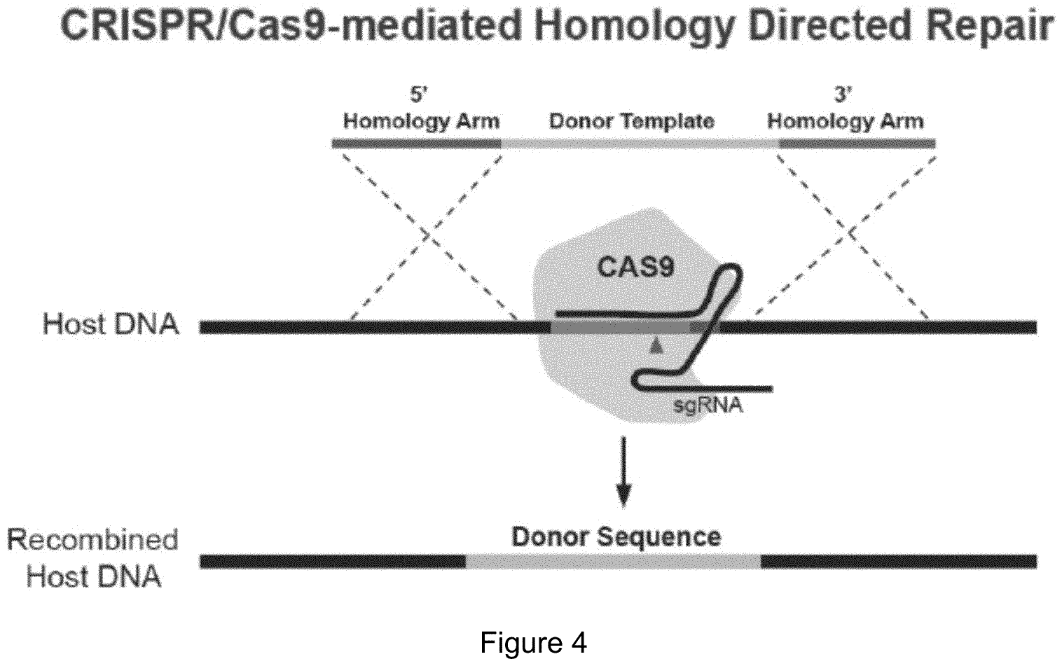

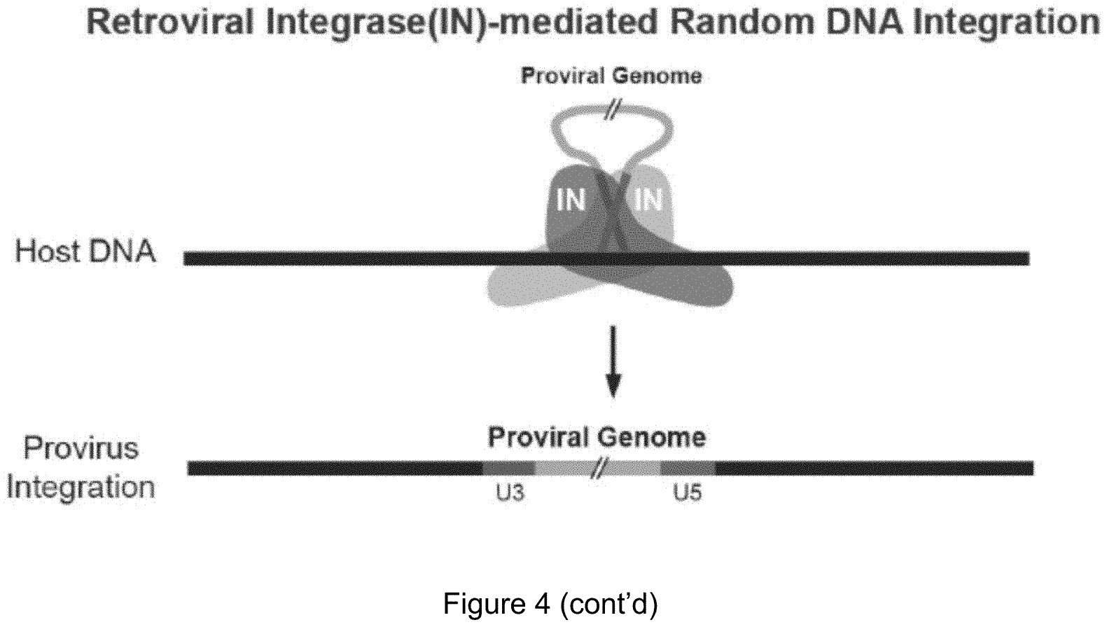

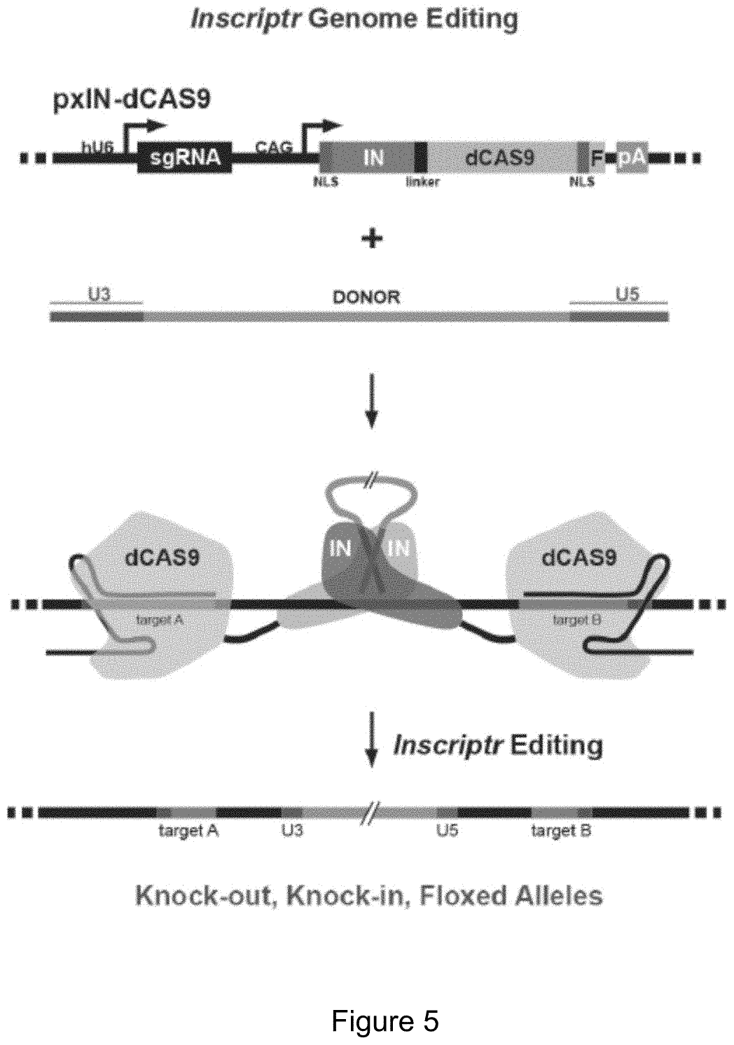

To quantify and characterize IN-dCas9 mediated integration in mammalian cells, a plasmid-based reporter assay was designed that utilizes the blue chromoprotein from the coral Acropora millepora (amilCP), which produces dark blue colonies when expressed in Escherichia coli (FIG. 6). Disruption of the amilCP open reading frame abolishes blue protein expression, which can be used as a direct readout for targeting fidelity and as a target DNA for Integrase-Cas-mediated integration. Single guide-RNA (sgRNA) target sequences were designed with a ‘PAM-out’ orientation separated by 16 bp spacer sequence, to promote efficient dimerization of the N-terminal dCas9 fusion protein at target DNA (FIG. 4).

### Generation of a Viral-End Donor Sequences for Integrase-Cas-Mediated Integration.

To construct a targeting vector that could be used to generate donor sequences for Integrase-Cas-mediated integration, the 30 base pairs encompassing the U3 and U5 HIV termini were subcloned into pCRII (FIG. 6). To facilitate subcloning of donor sequences, a multiple cloning site containing 9 unique restriction enzymes was included between U3 and U5. Since U3 and U5 share the same 3 nucleotides at their termini (ACT and AGT respectively) additional half-site sequences were included to generate ScaI restrictions sites at each end that could be used to generate bluntend donor sequences from the plasmid backbone (FIG. 6). Additionally, flanking Type IIS restriction enzyme sites were included for FauI, which cuts and leaves a two 5′ nucleotide overhang, mimicking the 3′ pre-processed viral end with exposed CA dinucleotide (FIG. 6). To aid in the gel purification and separation of FauI-digested templates from plasmid backbone, multisite directed mutagenesis was used to remove the six FauI sites present in the pCR II plasmid backbone.

### Protocol: Preparing INsrt Donor Templates for Transfection

1) Set up restriction digest of INsrt plasmid DNA

2) Restriction digest reaction

3) Gel purify the donor template from backbone DNA

4) Eluted Donor DNA for transfection.

Integrase-Cas-Mediated Integration of Donor Sequences into Plasmid DNA in Mammalian Cells.

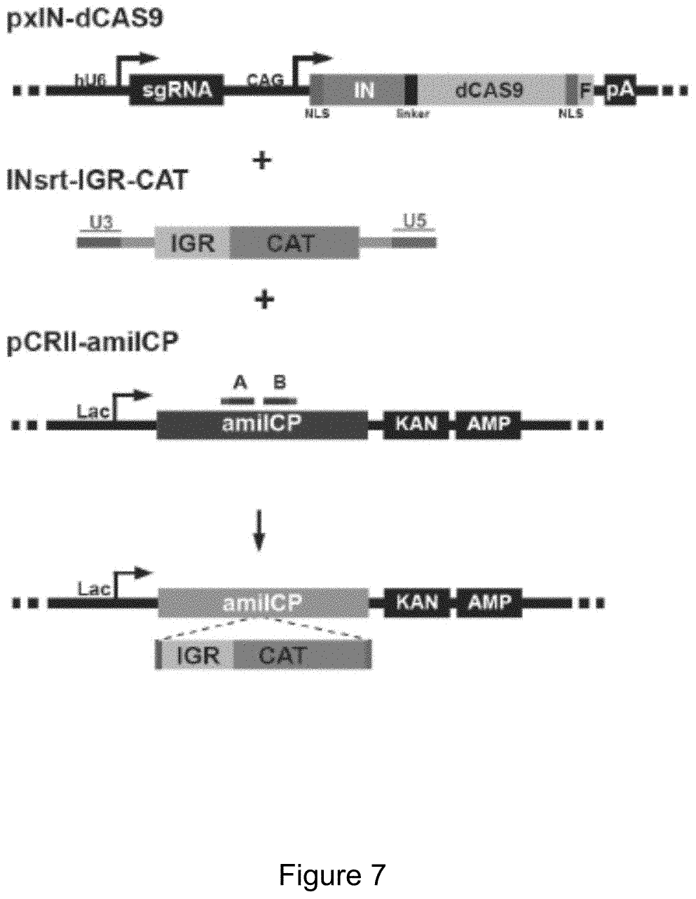

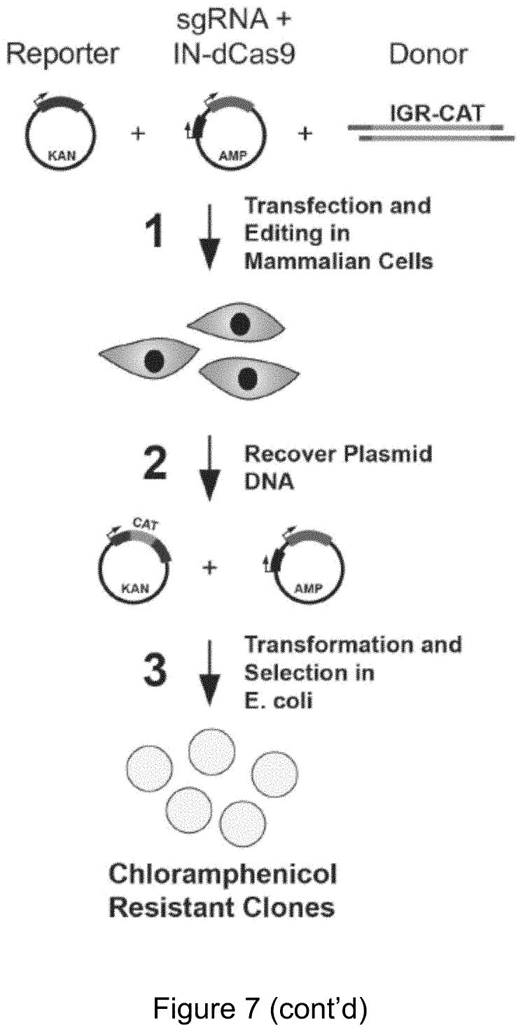

To allow for positive selection of concerted IN-dCas9-mediated integration, a INsrt donor vector was designed carrying the chloramphenicol resistance gene (CAT), which is not present in the reporter of expression plasmids (FIG. 7). The IGR IRES from the Plautia stali intestine virus (PSIV) was included in front of the CAT gene, which can initiate translation in both prokaryote and eukaryote cells, to aid in translation at multiple sites of integration. Templates containing the chloramphenicol resistance gene and viral termini were digested using either ScaI (Blunt ends) or FauI (processed ends) and gel purified from plasmid backbone DNA. Co-transfection of the INsrt templates, the IN-dCas9 vectors targeting the amilCP sequence were co-transfected into Cos7 cells (FIG. 7). After 48 hours, total plasmid DNA was recovered using column purification and transformed into E. coli. Chloramphenicol resistance clones were observed for both full length IN and INDC-dCas9 fusion proteins. Sequencing of the plasmids revealed the IG3-CAT plasmid sequence had integrated into the amilCP reporter. Interestingly, the use of FauI digested donor sequences, which mimic pre-3′processing of viral DNA ends, resulted in twice as many chloramphenicol resistance clones compared to ScaI digested blunt-end templates. Integrase-Cas-mediated integration contained hallmarks of HIV IN lentiviral integration, including a 5 base pair repeat of host DNA flanking the integration site. Interestingly, the integration site did not occur between the two sgRNA target sites but occurred on either side of the amilCP target sequence.

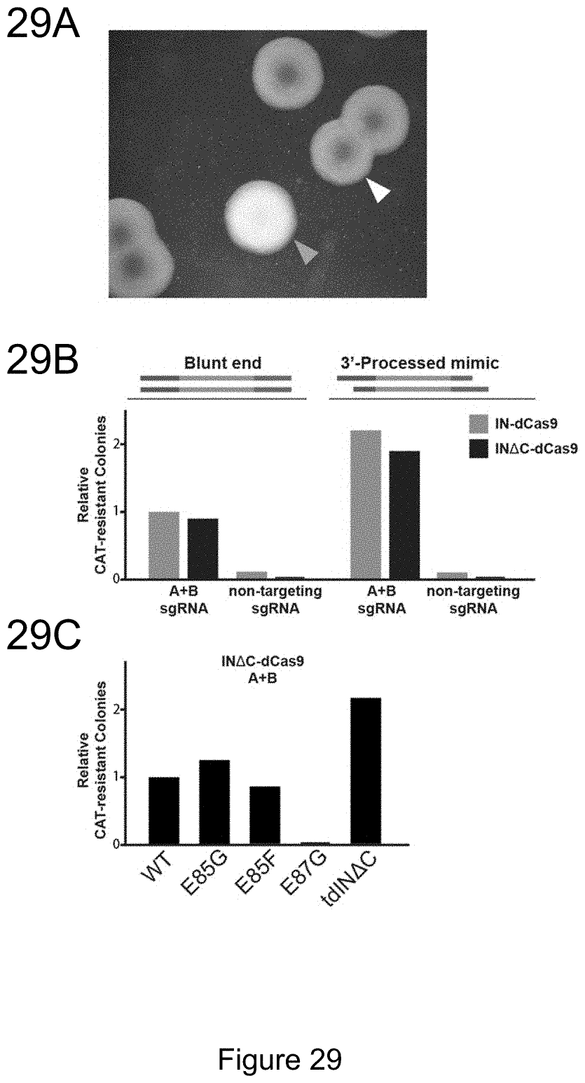

Integration of Insrt IGR-CAT donor template with either blunt ends (ScaI cleaved) or 3′ Processing mimic (FauI cleaved) ends into pCRII-amilCP reporter in mammalian cells. Interestingly, deletion of the C-terminal non-specific DNA binding domain, as a fusion to dCas9, does not inhibit Integrase-Cas mediated integration. Use of ends that mimic 3′ Processing show ˜2 fold increase in CAT resistant clones. (FIG. 29B) Dimerization inhibiting mutations (E85G and E85F) do not disrupt Integrase-Cas-mediated integration using double guide-RNA targeted integration of IGR-CAT donor template into amilCP. However, the IN E87G mutation cannot be rescued by paired targeting sgRNAs. Interestingly, a tandem INΔC fusion to dCas9 (tdINΔC-dCas9) shows ˜2 fold enhanced integration (FIG. 29C).

Protocol: Integrase-Cas-Mediated Integration of Donor Sequences into Plasmid DNA in Mammalian Cells
1) Co-transfect the multicistronic sgRNA and IN-dCas9 plasmid, bacterial amilCP reporter plasmid and INsrt donor template into mammalian (ex. Cos7) cells.
   a. Set up transfection reaction immediately before plating cells.
   b. Harvest and plate and transfect cells
2) Recover plasmid DNA from transfected cells:
3) Transform recovered plasmid DNA into chemically competent E. coli.
   Generation of a CMV-GFP Stable Mammalian Cell Line for Integrase-Cas-Mediated Integration into Genomic DNA.

A stable GFP reporter cell line was generated that can be used to quantify and characterize the fidelity of individual integration events in mammalian cells (FIG. 3). A plasmid encoding GFP under the control of the human CMV promoter (pcDNA3.1-GFP) was linearized and transfected into Cos7 cells and stable clones were selected using G418 and serial dilution. This artificial locus allows for robust gene expression which can be targeted for disruption without compromising the normal cell viability, which otherwise could occur when targeting an essential host gene.

Integrase-Cas-Mediated Integration of Donor Sequences into Mammalian Genomic DNA.

To quantify integration events at the CMV-GFP locus, a donor template was constructed containing an IGR-mCherry-2A-puromycin-pA cassette and paired guide-RNAs targeting the GFP coding sequence (FIG. 3). Integration of the donor cassette into the CMV-GFP locus will drive mCherry expression and disrupt GFP expression and provide resistance to the antibiotic puromycin. After transfection and 48 hours of culture, mCherry-positive cells were observed, some of which still contained weak but detectable levels of GFP expression (FIG. 3).

### Integrase-Cas-Mediated Integration of Donor Sequences at an Endogenous Locus.

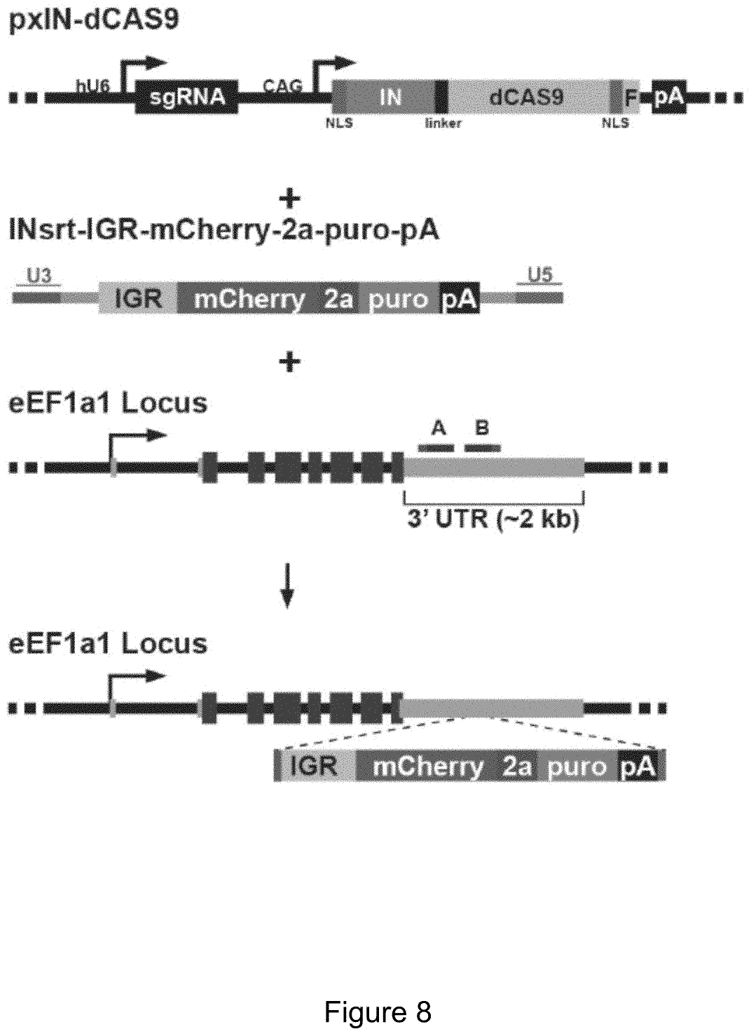

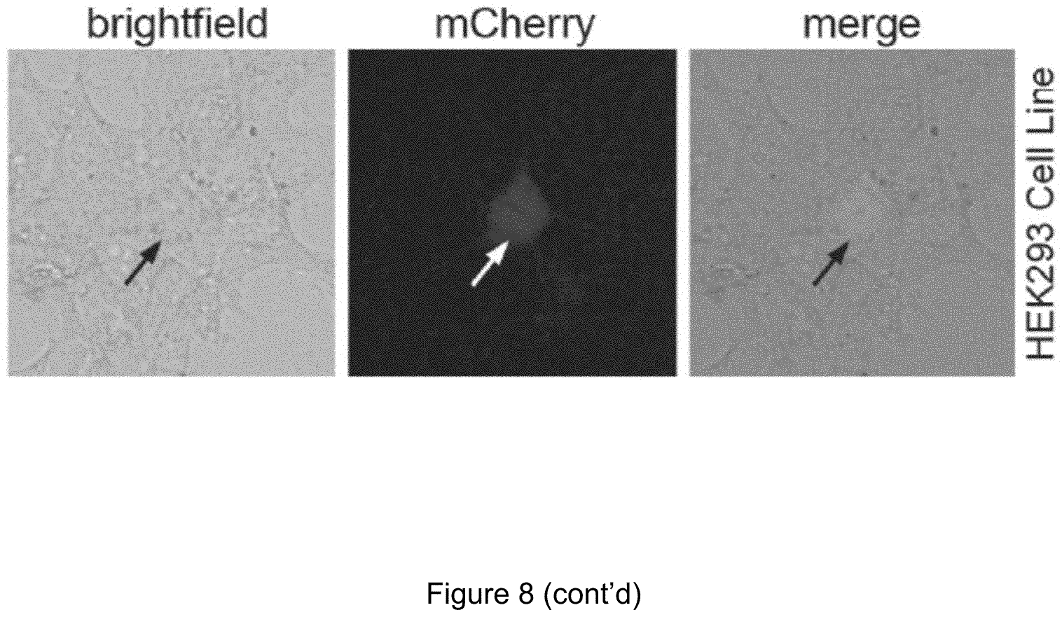

A targeting strategy was designed and guide-RNAs specific the 3′UTR of the human EF1-alpha locus were selected to knock-in the IGR-mCherry-2A-puromycin-pA cassette into the human HEK293 cell line (FIG. 8). The 3′UTR was targeted to allow for expression of the IGR-mCherry cassette, while not disrupting the open reading frame of the EF1-alpha expression. After transfection and 48 hours of culture, mCherry-positive cells were observed in culture (FIG. 8).

Protocol: Integrase-Cas-Mediated Integration of Donor Sequences into Mammalian Genomic DNA
1) Co-transfect plasmids encoding sgRNAs, IN-dCas9 and INsrt donor template 1:1:1 into mammalian cells (COST, HEK293, etc) using Fugene6 or Lipofectamine2000.
   a. Harvest, plate, and transfect cells.
2) Antibiotic Selection for integrated sequences
   a. Wash cells with and plate in 10 mls of media containing antibiotic selection
   b. Culture cells, then generate clones.

### Directional Editing.

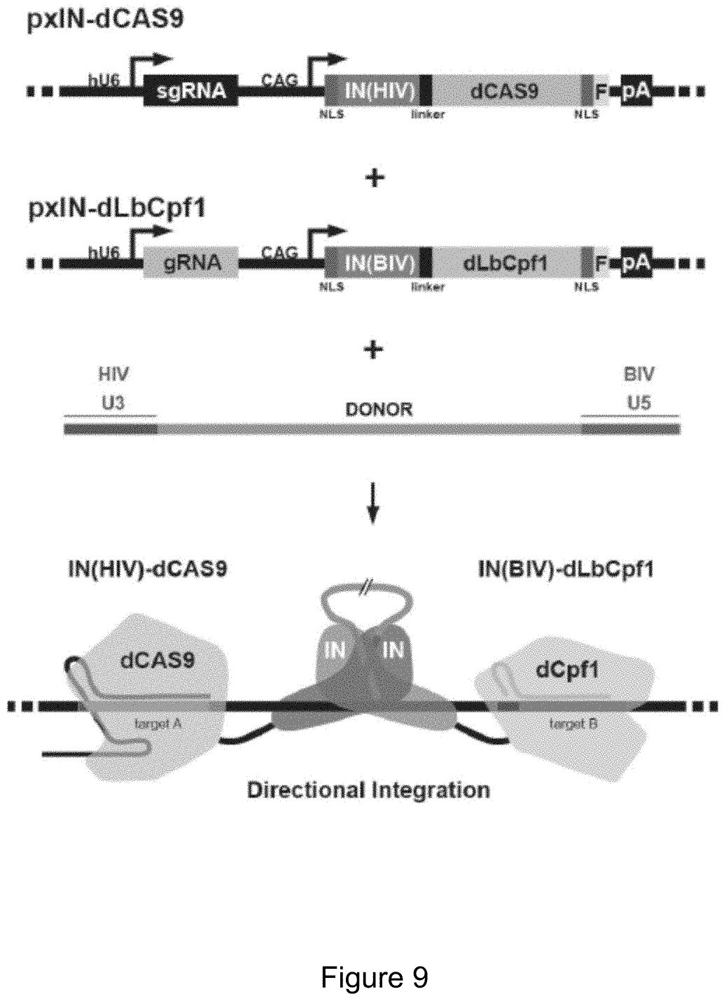

IN-mediated integration of DNA sequences can occur in either direction in a target DNA sequence. Utilizing different combinations of Cas and IN retroviral class proteins provides the ability to promote direction editing. For example, a fusion of IN from BIV (Bovine Immunodeficiency virus, or other HIV related virus) fused to catalytically dead LbCpf1 (LbCpf1) allows for binding to a specific target sequence utilizing a Cpf1-specific guide-RNA. Utilizing a donor sequence containing both HIV and BIV terminal sequences lock binding to a single orientation with the target DNA. (FIG. 9).

### Multiplex Genome Editing for the Generation of Floxed Alleles.

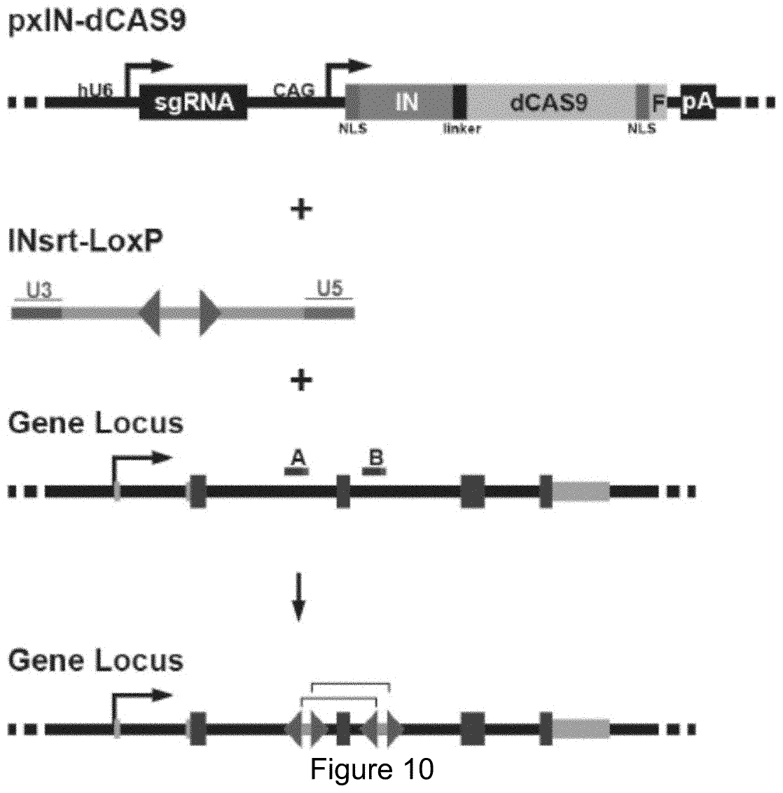

The incorporation of flanking LoxP (Floxed) sequences around a gene of interest allows for CRE-mediated recombination and conditional mutagenesis. Current methods to generate Floxed alleles using CRISPR-Cas9 are inefficient. The most widely utilized approach is to use two guide-RNAs to induce DNA cleavage at flanking target sequences and Homology Direct Repair to insert ssDNA templates containing LoxP sequences. However, when using double sgRNAs to induce cleavage, the most favorable reaction is the deletion of intervening sequence, resulting in global gene deletion. The use of Integrase-Cas-mediated gene insertion provides an alternative and more efficient approach for tandem insertion of DNA sequences if IN-mediated strand transfer with host DNA does not allow for efficient deletion of intervening sequences. Since IN-mediated integration may occur in either the direction, Integration of a sequence containing inverted LoxP sequences allows for recombination of flanking LoxP sequences (FIG. 10).

### Example 5: Identification and Activity of Ty1 NLS-Like Sequences

The integrase enzyme from the yeast Ty1 retrotransposon contains a non-classical bipartite nuclear localization signal, comprised of tandem KKR motifs separated by a larger linker sequence. Previous studies in yeast have demonstrated the necessity of these basic motifs for nuclear localization and Ty1 transposition (Kenna et al., 1998, Mol Cell Biol 18, 1115-1124; Moore et al., 1998, Mol Cell Biol 18, 1105-1114). Ty1 transposition is absolutely dependent on the presence of the Ty1 NLS, and interestingly, a classic NLS is insufficient to recapitulate Ty1 NLS activity required for transposition. Interestingly, additional yeast proteins share this tandem KKR motif, which may serve to function as an NLS given that many of these proteins are nuclear localized (Kenna et al., 1998, Mol Cell Biol 18, 1115-1124).

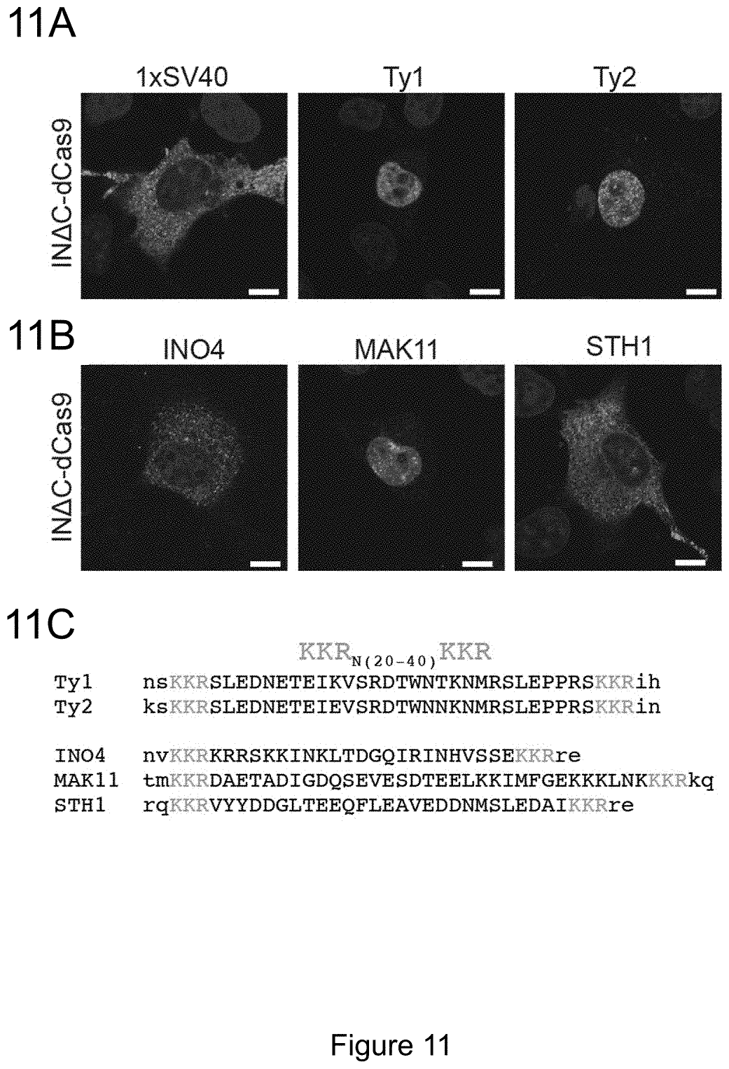

As demonstrated in Example 1, the yeast Ty1 NLS provides robust nuclear localization of Cas proteins and Cas-fusion proteins in mammalian cells. To determine if this activity is a unique feature of the Ty1 NLS, it was tested whether the closely related NLS from Ty2 Integrase and other yeast Ty1 NLS-like motifs were sufficient to localize an Integrase-dCas9 fusion protein (INΔC-Cas9) to the nucleus in mammalian cells. Interestingly, the Ty2 NLS, which is highly conserved to the Ty1 NLS, was equally as efficient for nuclear localization as the Ty1 NLS (FIG. 11). Fusion of three different Ty1 NLS-like sequences identified in yeast (Kenna et al., 1998), which diverge from Ty1/Ty2 NLS sequences, showed either robust NLS activity (MAK11) or no apparent NLS activity (INO4 and STH1). The MAK11 sequence is derived from a yeast nuclear protein, which also occurs at the C-terminus of the protein were further screen, suggesting this sequence indeed functions as NLS. All proteins in the SWISS-PROT Protein Sequence Databank using the motif KKR_{N20-40}KKR, which identified a large number of potential Ty1 NLS-like sequences across diverse species (SEQ ID NOs:275-887). These data demonstrate that other Ty1 NLS-like sequences may have robust NLS activities and maybe useful for localization of proteins (including Cas and Cas-fusion proteins) in dividing and non-dividing eukaryotic cells.

### Example 6: Enhanced CRISPR-Cas9 DNA Editing with the Ty1 NLS

CRISPR-Cas DNA cleavage systems are derived from bacteria and Cas proteins are both large and lack intrinsic mammalian nuclear localization signals (NLSs), preventing their efficient nuclear localization in mammalian cells. Previously it has been shown that the addition of two classical nuclear localization signals (an N-terminal SV40 and C-terminal nucleoplasmin (NPM) bi-partite NLS) were required for efficient nuclear localization and editing of DNA by CRISPR-Cas9 in mammalian cells (Cong et al., 2013, Science 339, 819-823). Due to the robust nature of the non-classical yeast retrotransposon Ty1 NLS for localizing Cas fusion proteins in mammalian cells (Example 1), it was tested whether the Ty1 NLS could also function to enhance the editing efficiency of traditional CRISPR-Cas9 in mammalian cells.

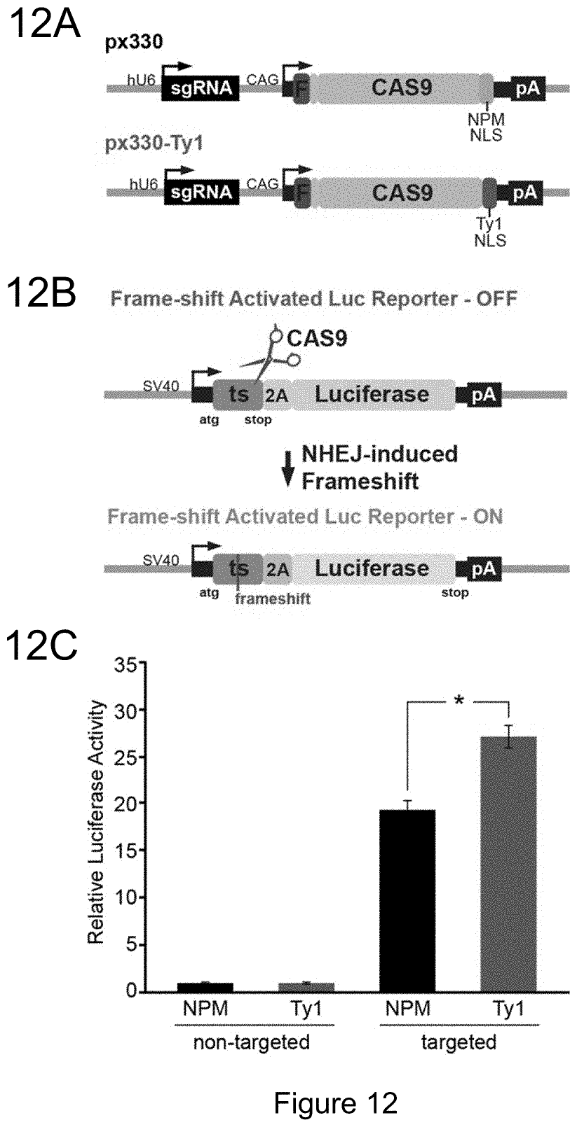

To determine if Ty1 enhances CRISPR-Cas9 editing, an existing CRISPR-Cas9 expression plasmid (px330) was modified by replacing the C-terminal NPM NLS with the non-classical Ty1 NLS (px330-Ty1) (FIG. 12A). Next, a frameshift-responsive luciferase reporter was generated, which encodes an out-of-frame luciferase coding sequence downstream of a target sequence (ts) (FIG. 12B). For this reporter assay, cleavage near the target sequence and imperfect repair by the cellular non-homologous end joining (NHEJ) pathway can induce nucleotide insertions or deletions which have the potential to re-frame the luciferase coding sequence and result in luciferase expression.

Co-expression of the Luciferase reporter with a vector encoding Cas9 containing the NPM NLS and a single guide-RNA specific to a 20 nucleotide target sequence resulted in a ˜20-fold increase in luciferase activity over background, relative to a non-targeting guide-RNA (FIG. 12C). Notably, expression of Cas9 containing the Ty1 NLS resulted in a significant (˜44%) enhancement in reporter activity in COS-7 cells, compared to Cas9 containing the NPM NLS (FIG. 12C).

### Example 7: Genome Targeting Strategies for Editing

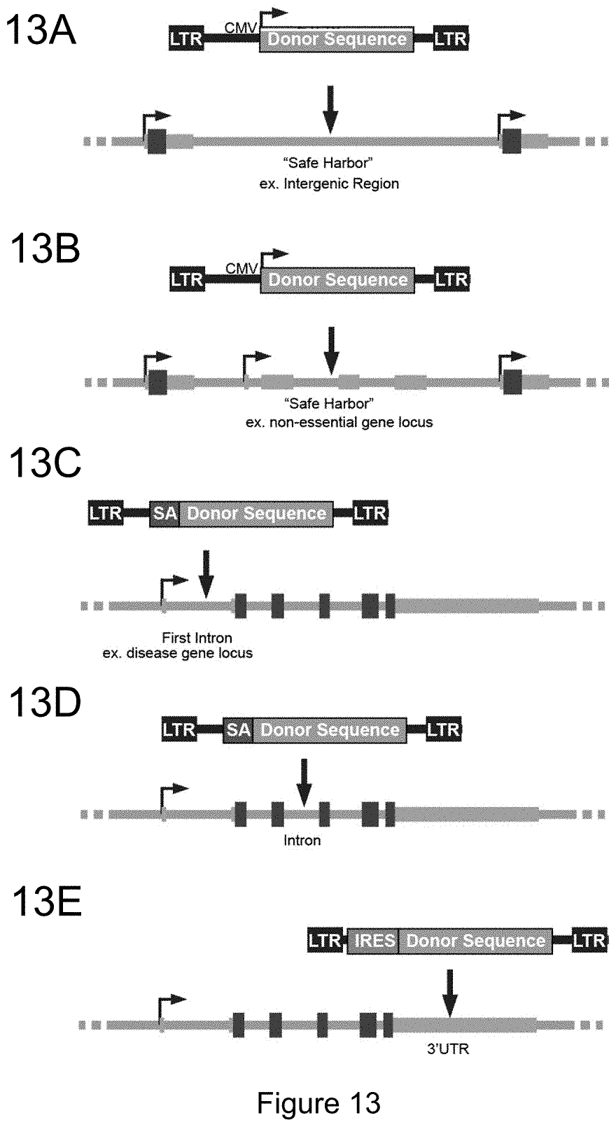

Targeted integration of DNA donor sequences using an Integrase-DNA-binding fusion protein can be targeted to different locations within the genome depending upon the desired outcomes. For example, therapeutic DNA Donor sequences consisting of a gene expression cassette (ex, promoter, gene sequence and transcriptional terminator) may be targeted to ‘safe harbor’ locations (for review and list of safe harbor sites in the human genome, see Pellenz et al., 2019, Hum Gene Ther 30, 814-828), which would allow for expression of a therapeutic gene without affecting neighbor gene expression. These may include intergenic regions apart from neighbor genes ex. H11, or within ‘non-essential’ genes, ex. CCR5, hROSA26 or AAVS1 (FIGS. 13A and 13b).

To restore expression of a disease causing gene mutation, targeted integration of a therapeutic gene sequence into the endogenous disease gene locus may be advantageous, since this locus is already defective and the spatial and temporal expression of this locus is under endogenous regulatory control. In one iteration, a DNA donor sequence encoding a therapeutic gene containing a splice acceptor could be integrated into the first intron of the endogenous gene locus, such that splicing would 1) allow for expression of the introduced gene sequence and 2) prevent downstream expression of the mutated sequence (due to termination from an integrated poly(A) sequence or LTR sequence (FIG. 13C). Smaller DNA donor sequences could be delivered or expressed if this is targeted to a downstream intron (FIG. 13D).

Targeted insertion of a DNA donor sequence containing an IRES sequence into a 3′ untranslated region (3′UTR) of a gene may be beneficial in that this approach would allow for expression in the same spatial and temporal expression as the targeted locus and would be less likely to disrupt the targeted gene locus (FIG. 13E).

### Example 8: Targeted Lentiviral Integration into Mammalian Genomes Using CRISPR-CAS

The data presented herein demonstrates three different approaches for the delivery and targeted integration of lentiviral donor sequences into mammalian genomes.

### Lentivirus Life Cycle

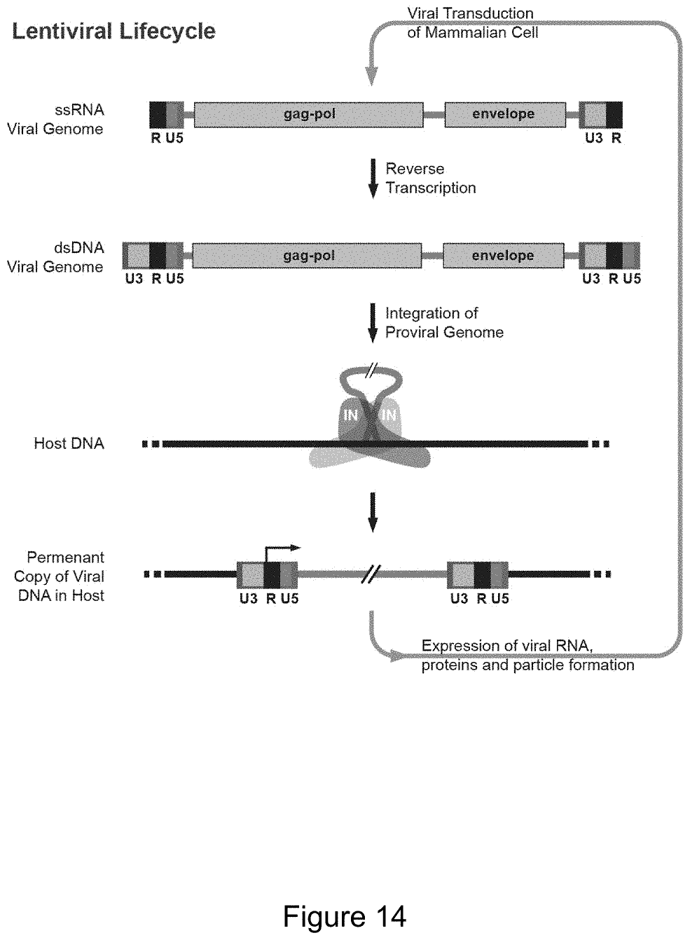

Lentiviruses are single-stranded RNA viruses which integrate a permanent double-stranded DNA(dsDNA) copy of their proviral genomes into host cellular DNA (FIG. 14). Lentiviral genomes are flanked by long terminal repeat (LTR) sequences which control viral gene transcription and contain short (˜20 base pair) sequence motifs at their U3 and U5 termini required for proviral genome integration. Subsequent to viral infection, lentiviral RNA genomes are copied as blunt-ended dsDNA by viral-encoded reverse transcriptase (RT) and inserted into host genomes by Integrase (IN). IN consists of three functional domains which are essential for IN activity, including a C-terminal domain that binds non-specifically to DNA (CTD). IN-mediated insertion of retroviral DNA occurs with little DNA target sequence specificity and can integrate into active gene loci, which can disrupt normal gene function and has the potential to cause disease in humans. This limits the utility of lentiviral vectors for gene therapy, despite the benefits of a large sequence carrying capacity.

### Genome Editing

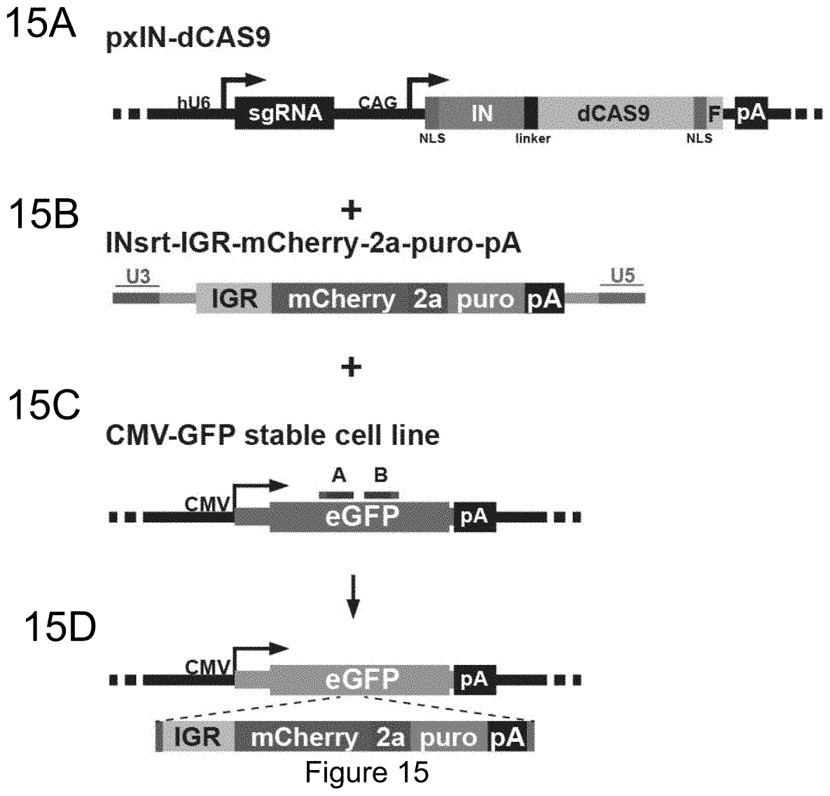

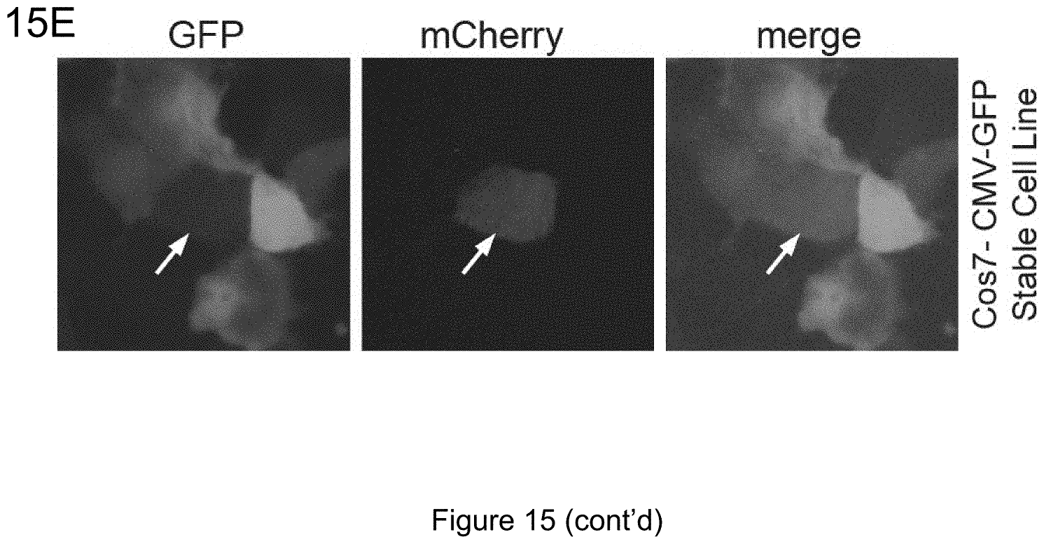

CRISPR-Cas9 allows for programmable DNA targeting by utilizing short single guide-RNAs to recognize and bind DNA. Catalytically inactive Cas9 (dCas9) retains the ability to target DNA and has been recently repurposed as a programmable DNA binding platform for diverse applications for genome interrogation and regulation. As demonstrated in example 1, fusion of lentiviral Integrase to dCas9 is sufficient to insert donor DNA sequences containing short viral termini to target sequences using CRISPR guide-RNAs in mammalian cells (FIG. 15). To monitor Integrase-Cas-mediated integration in mammalian cells, donor vector were generated containing the IGR IRES sequence followed by an mCherry-2a-puromycin gene and an SV40 polyadenylation sequence (FIG. 15B). sgRNAs targeting a stable human CMV-eGFP stable cell line in COS-7 cells were designed (FIGS. 15C and 15D). The hCMV-eGFP stable transgene provided a heterologous target sequence which can be used to determine editing at a robustly expressed but non-essential expression locus. Donor mCherry-2a-puro templates were purified and co-transfected with sgRNAs and IN-dCas9 into the GFP stable cells and cultured for 48 hours. After 48 hours, mCherry-positive cells were visible in culture and replaced the GFP positive signal (FIG. 15E). Incorporating editing components (Integrase-CRISPR-Cas9 fusions) into lentiviral particles allows for targeted and readily programmable lentiviral genome integration into host DNA, thereby eliminating a major limitation of lentiviral gene therapy (i.e. non-specific lentiviral integration). This approach is useful for both basic research and therapeutic applications.

### Lentiviral Gene Delivery Systems

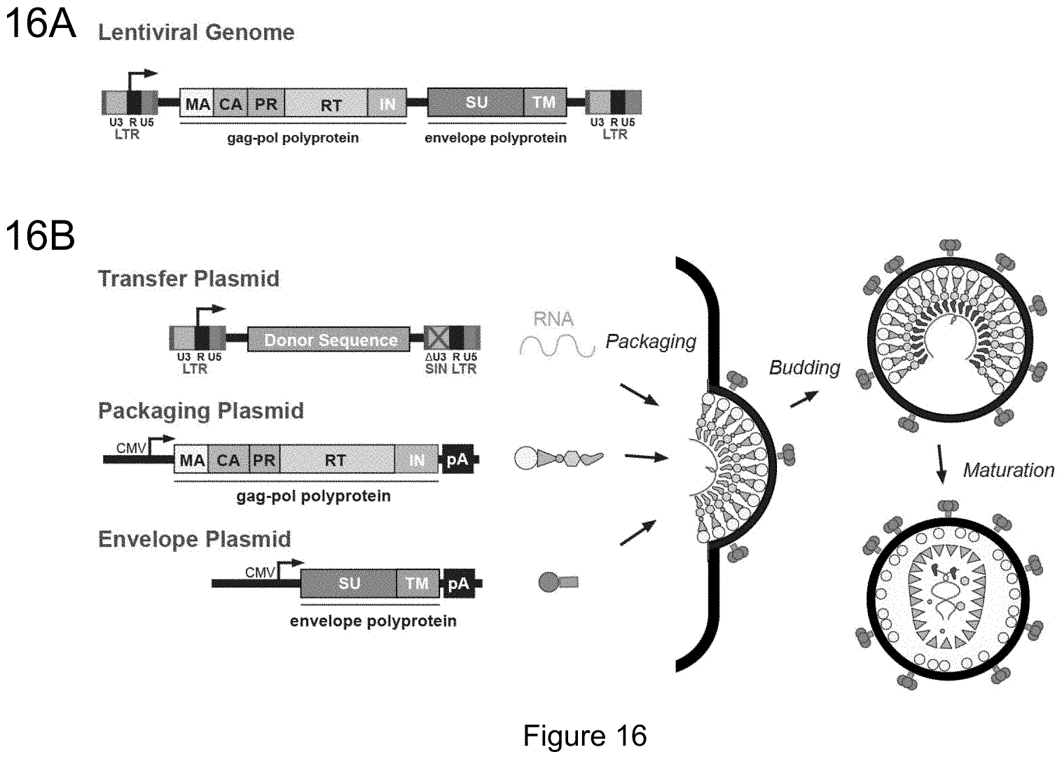

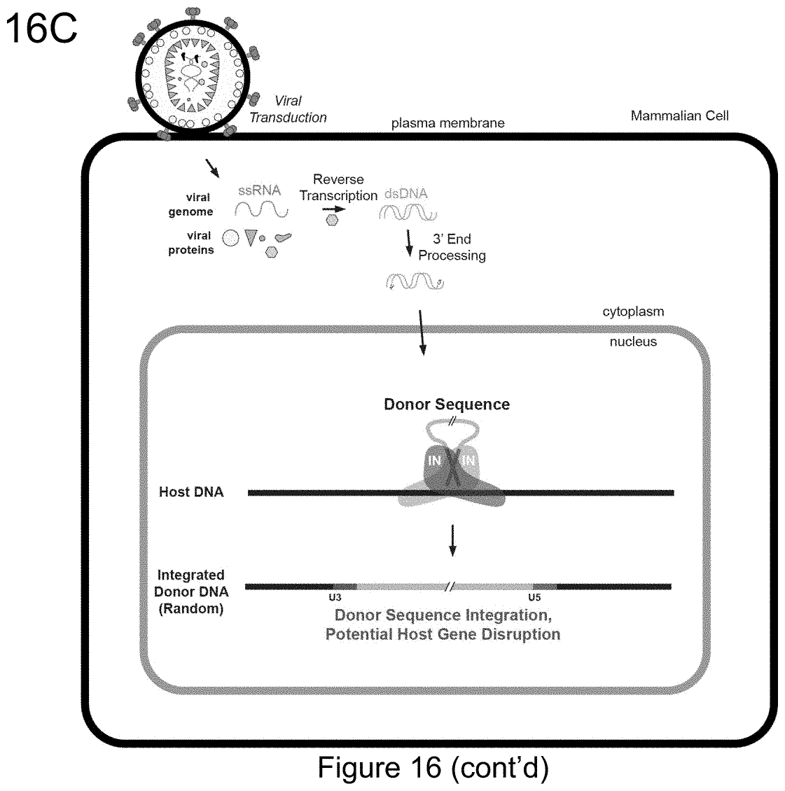

Lentiviral vectors have been adapted as robust gene delivery tools for research applications (FIG. 16). Lentiviral structural and enzymes proteins are transcribed and translated as large polyproteins (gag-pol and envelope) (FIG. 16A). Upon incorporation into budding viral particles, the polyproteins are processed by viral protease into individual proteins. For lentiviral vector gene expression systems, theses polyproteins are removed from the viral genome and expressed using separate mammalian expression plasmids (FIG. 16B). Donor DNA sequences of interest can then be cloned in place of viral polyproteins between the flanking LTR sequences. Co-transfection of these vectors in mammalian cells allows for the formation of lentiviral particles capable of delivering and integrating the encoded donor sequence, however do not require the coding information for Integrase and other viral proteins necessary for subsequent viral propagation (FIG. 16B). Lentiviral particles are a natural vector for the delivery of both viral proteins (ex. integrase and reverse transcriptase) and dsDNA donor sequences, which contain the necessary viral end sequences required for integrase-mediated insertion into mammalian cells (FIG. 16C).

Packaging the Integrase-dCas9 Fusion Protein into Lentiviral Particles.

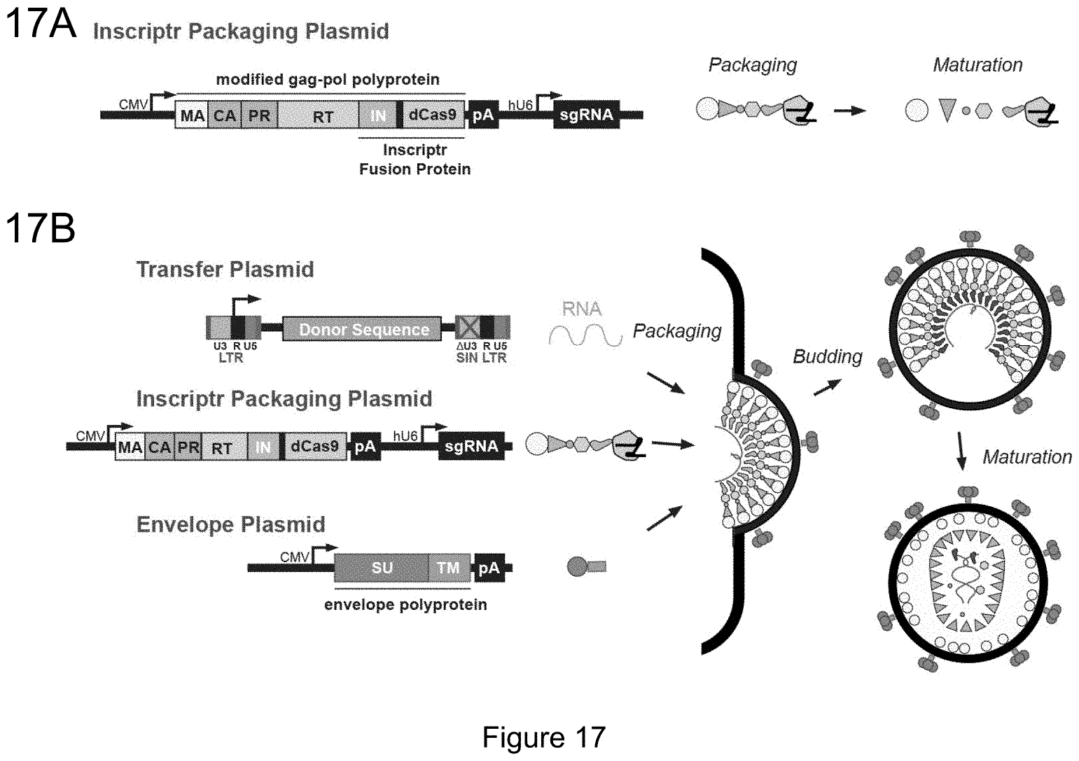

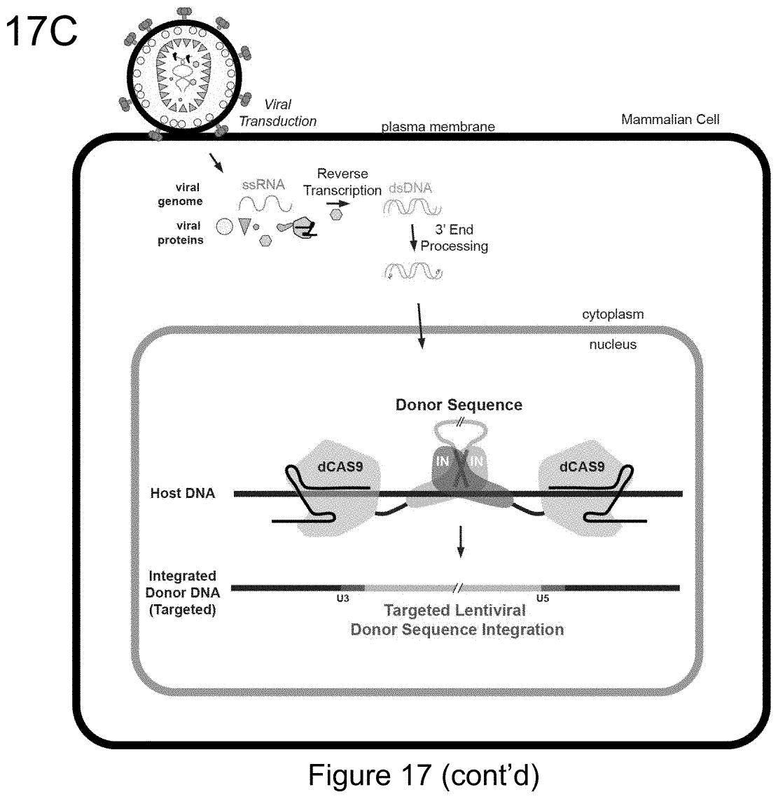

Existing lentiviral delivery systems can be modified to incorporate editing components for the purpose of targeted lentiviral donor template integration for genome editing in mammalian cells (FIGS. 17-20). Described herein are three different approaches for the delivery and targeted integration of lentiviral donor sequences into mammalian genomes.

The first approach is to incorporate dCas9 directly as a fusion to Integrase (or to Integrase lacking its C-terminal non-specific DNA binding domain, INΔC) within a lentiviral packaging plasmid (ex. psPax2) encoding the gag-pol polyprotein (FIG. 17A). In this approach, the modified gag-pol polyprotein is translated with other viral components as a polyprotein, loaded with guide-RNA and packaged into lentiviral particles (FIG. 4B). The Integrase-dCas9 fusion protein retains the sequences necessary for protease cleavage (PR), and thus is cleaved normally from the gag-pol polyprotein during particle maturation. Transduction of mammalian cells results in the delivery of viral proteins, including the IN-dCas9 fusion protein, sgRNA, and lentiviral donor sequence. Reverse transcription of the ssRNA genome by reverse transcriptase generates a dsDNA sequence containing correct viral end sequences (U3 and U5) which is then Integrated into mammalian genomes by the IN-dCas9 fusion protein.

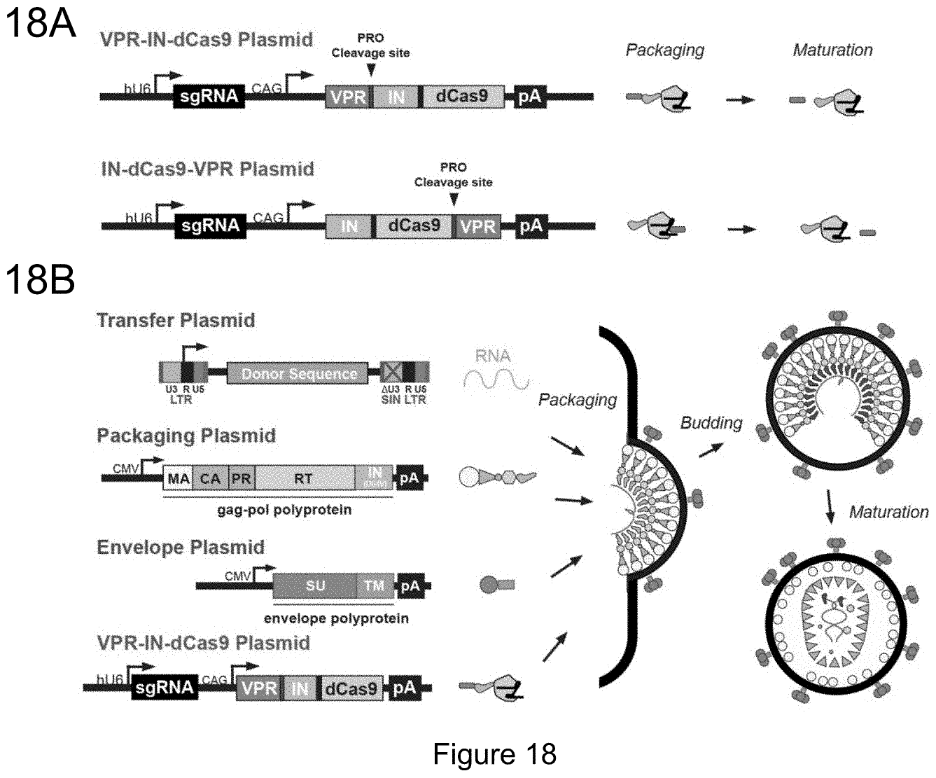

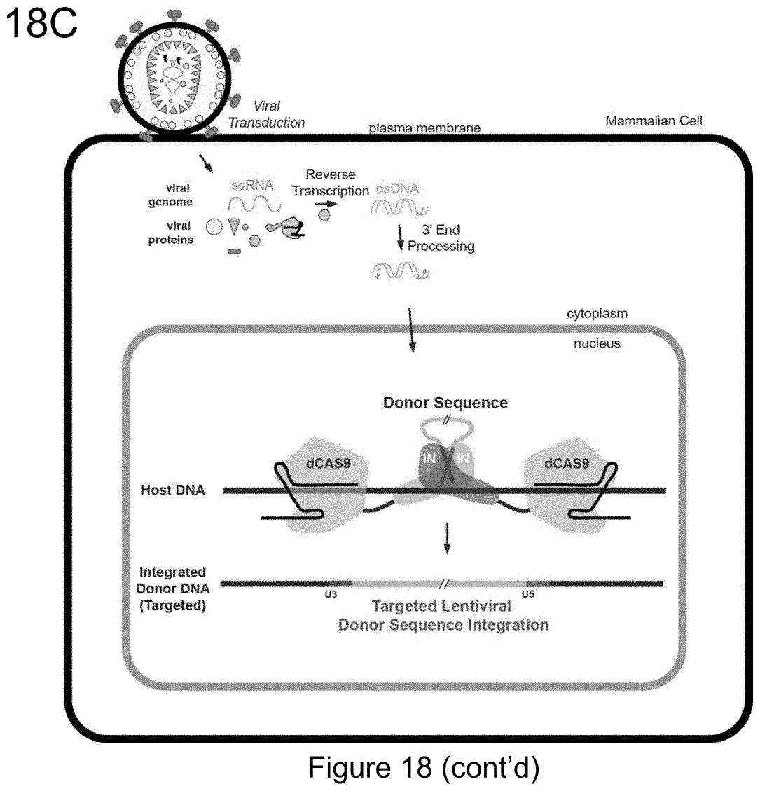

A second approach is to generate N-terminal and C-terminal fusions of Integrase-dCas9 with the HIV viral protein R (VPR) (FIG. 18A). VPR is efficiently packaged as an accessory protein into lentiviral particles and has been used to package heterologous proteins (e.x. GFP) into lentiviral particles. A viral protease cleavage sequence is included between VPR and the IN-dCas9 fusion protein, so that after maturation, the IN-dCas9 is freed from VPR (FIG. 18A). Co-transfection of packaging cells with lentiviral components generates viral particles containing the VPR-IN-dCas9 protein and sgRNA. The packaging plasmid required for viral particle formation (ex. psPax2) contains a mutation within Integrase to inhibit its catalytic activity, thereby preventing non-mediated integration (FIG. 18B). Upon viral transduction, the Integrase-dCas9 protein is delivered and mediate the integration of the lentiviral donor sequences (FIG. 18C). The benefit to delivery of the IN-dCas9 fusion and sgRNA as a riboprotein is that it is only transiently expressed in the target cell.

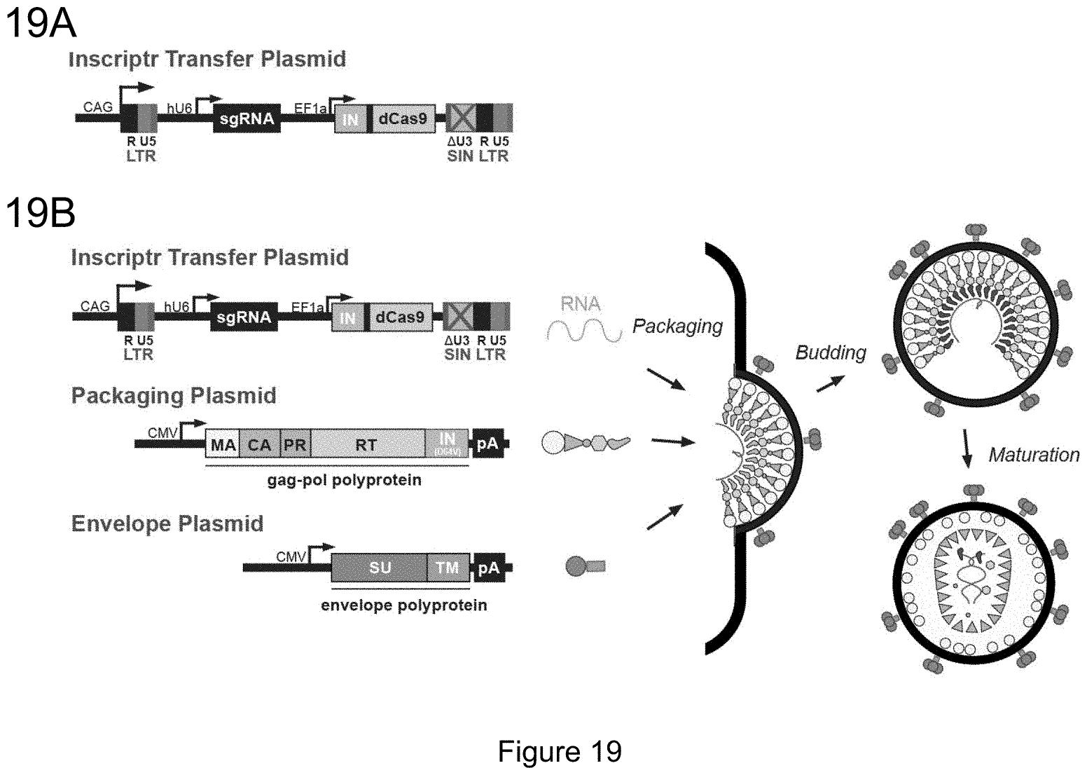

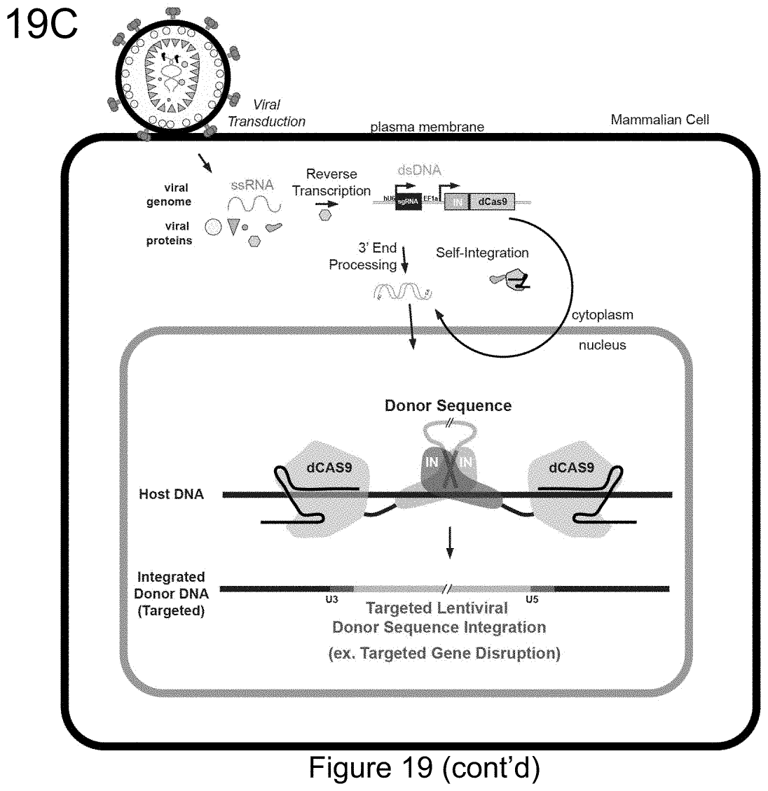

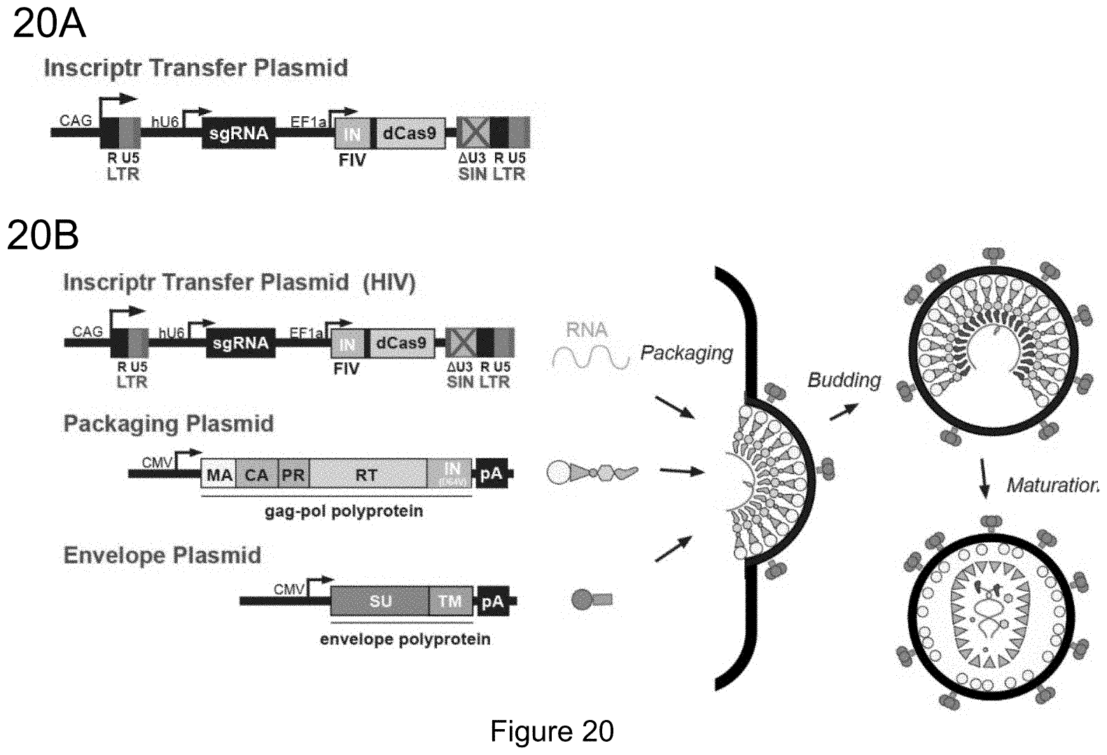

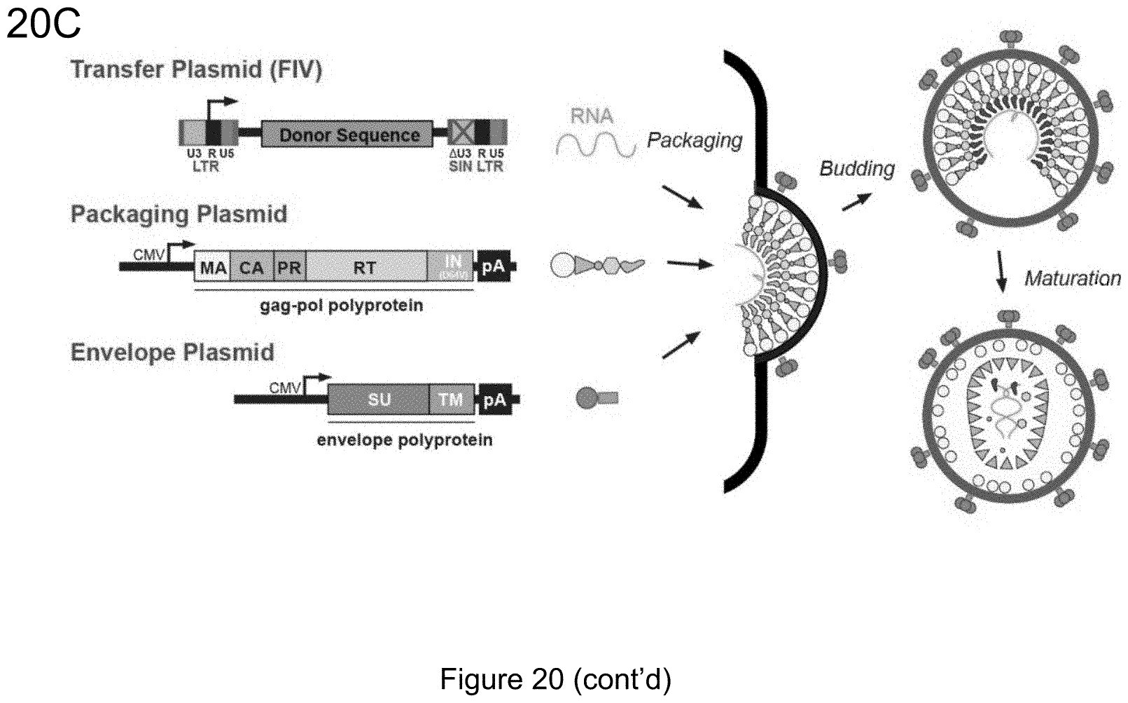

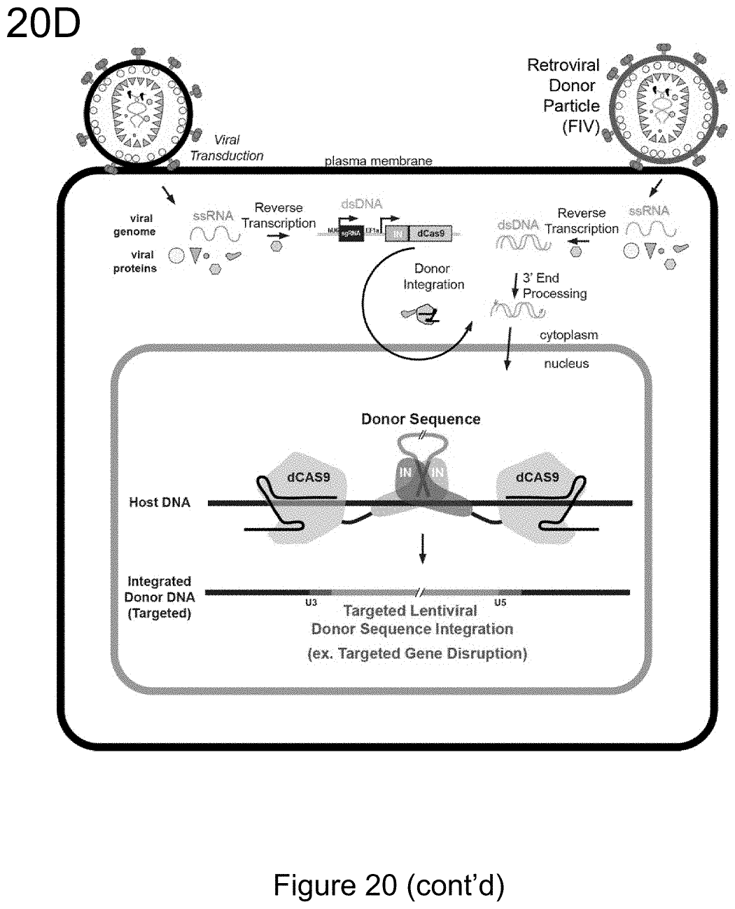

A third method is to incorporate the Integrase-dCas9 fusion protein and sgRNA expression cassettes directly within a lentiviral transfer plasmid, or other viral vector (such as AAV) (FIG. 19A). The transfer plasmid containing the IN-dCas9 fusion protein and sgRNA is co-transfected with packaging and envelope plasmids required to generate lentiviral particles. If using a lentivirus, the packaging plasmid contains a catalytic mutation within Integrase to inhibit non-specific integration (FIG. 19B). Upon transduction of a mammalian cell, expression of the IN-dCas9 fusion protein and sgRNA generate components capable of targeting its own viral donor vector for targeted integration (self-integration) (FIG. 19C). This method is used for targeted gene disruption or as a gene drive. Alternatively, co-transduction with an additional lentiviral particle encoding a donor sequence serves as the integrated donor template (FIG. 19). Prevention of self-integration of its own viral encoding sequence in this approach is achieved by using Integrase enzymes from different retroviral family members and their corresponding transfer plasmids. For example, an HIV lentiviral particle encoding an FIV IN-dCas9 fusion protein is utilized to integrate an FIV donor template encoded within an FIV lentiviral particle (FIG. 20).

Generation of a single locus, constitutively active, ubiquitous ROSA26^{mGFP/+} reporter mouse line

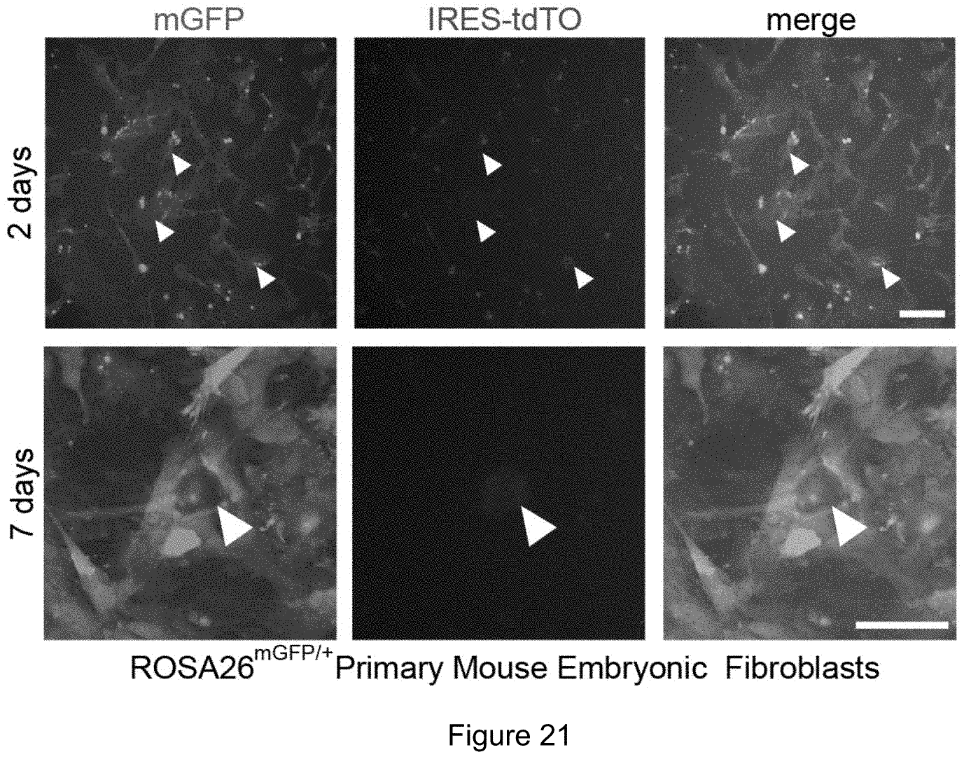

The ROSA26 mT/mG reporter mouse line (Jackson Labs, Stock #007576) contains a foxed, membrane localized tdTO (mT) fluorescent reporter cassette, which when recombined with a CRE recombinase, results in removal of a mT reporter and allows for expression of a membrane localized eGFP (mG) reporter. To generate a single locus, in vivo GFP reporter line, ROSA26 mT/mG mice were crossed with a universal CAG-CRE recombinase mouse to generate a constitutively and ubiquitously expressed ROSA26 mG reporter mouse. Isolation of mouse embryonic fibroblasts (MEFs) from heterozygous ROSA26^{mG/+} mice revealed robust membrane GFP expression in all cells in culture (FIG. 21). A similar strategy is utilized to generate a ubiquitous and constitutively active nuclear GFP reporter by recombining the ROSA26 nT/nG mouse strain (Jackson Labs, Stock #023035).

Packaging of Components into Lentiviral Particles for Targeted Integration into the ROSA-mGFP Locus.

For targeted integration of an IRES-tdTO sequence into the GFP coding sequence in ROSA26^{mG/+} MEFs, lentiviral particles were generated in a packaging cell line (Lenti-X 293T, Clontech). Lentiviral particles were generated by co-transfection of a lentiviral transfer plasmid encoding an IRES-tdTO fluorescent reporter between an 2^{nd} generation SIN lentiviral LTRs (Lenti-IRES-tdTO), an expression vector encoding a pantropic envelope protein (VSV-G), expression plasmid encoding inverted pair of GFP-targeting guide-RNAs, and a packing plasmid encoding an INΔC-dCas9 fusion in the context of the Gag-Pol lentiviral polyprotein in the psPax2 packing plasmid (INΔC-dCas9-psPax2). Lentiviral particles were harvested from supernatant, filtered using 0.45 μm PES filter.

### Targeted Lentiviral Integration in Mammalian Cells

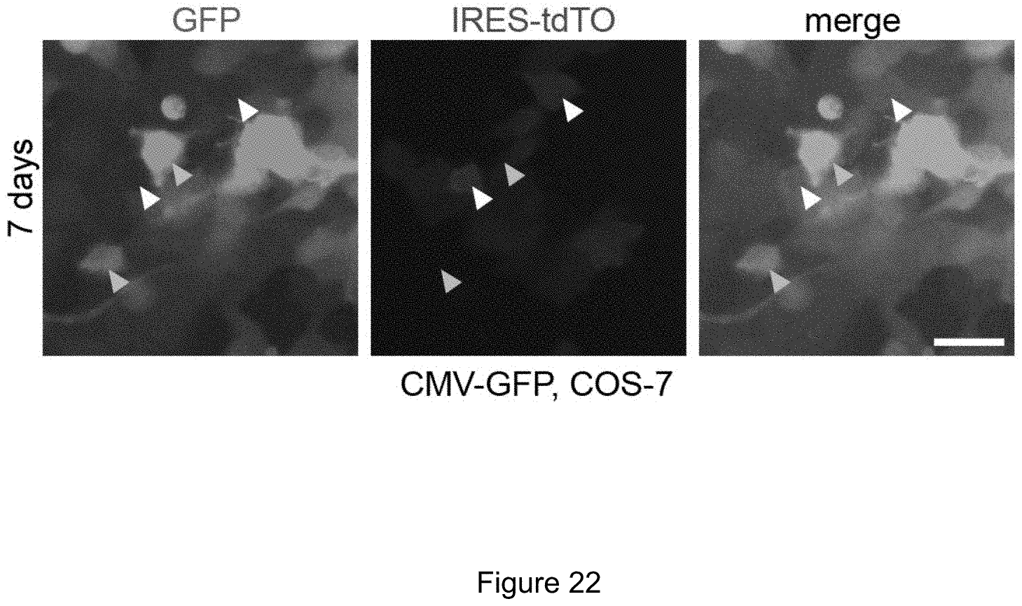

Incriptr-modified lentiviral particles were used to transduce ROSA26^{mG/+} MEFs in culture. After two days, ubiquitous red fluorescent protein expression was detectable in MEFs transduced with lentivirus encoding the IRES-tdTO reporter but retained GFP fluorescence. This initial broad expression is likely due to translation of the lentiviral IRES-tdTO encoded viral RNA and demonstrates that lentiviral packaging was not inhibited by modifications in the packaging plasmid (FIG. 21). For traditional lentiviral transduction, in the absence of viral integration, lentivirus transgene expression is not maintained. Remarkably, seven days post-transduction, tdTO red fluorescent cells were detectable in in culture, which now lacked green fluorescence in ROSA26^{mG/+} primary cells (FIG. 21) or when targeted into our previously described CMV-GFP COS-7 table cell line (FIG. 22). These data demonstrate that fusion of Integrase (lacking a C-terminal DNA binding domain) to catalytically dead Cas9 in the context of the Gag-Pol lentiviral polyprotein allows for lentiviral packaging, delivery and targeting of lentiviral encoded donor sequences in mammalian cells. Further, these data suggest that expression of guide-RNAs in lentiviral packaging cells are sufficient for incorporation into lentiviral particles, which may occur through the strong interaction with dCas9. Alternative approaches to deliver guide-RNAs into lentiviral particles may enhance targeted integration, for example, through constitutive expression of the guide-RNA(s) in the transfer plasmid, etc.

### Alternative DNA Binding Domains for Targeted Integration of Lentiviral Particles.

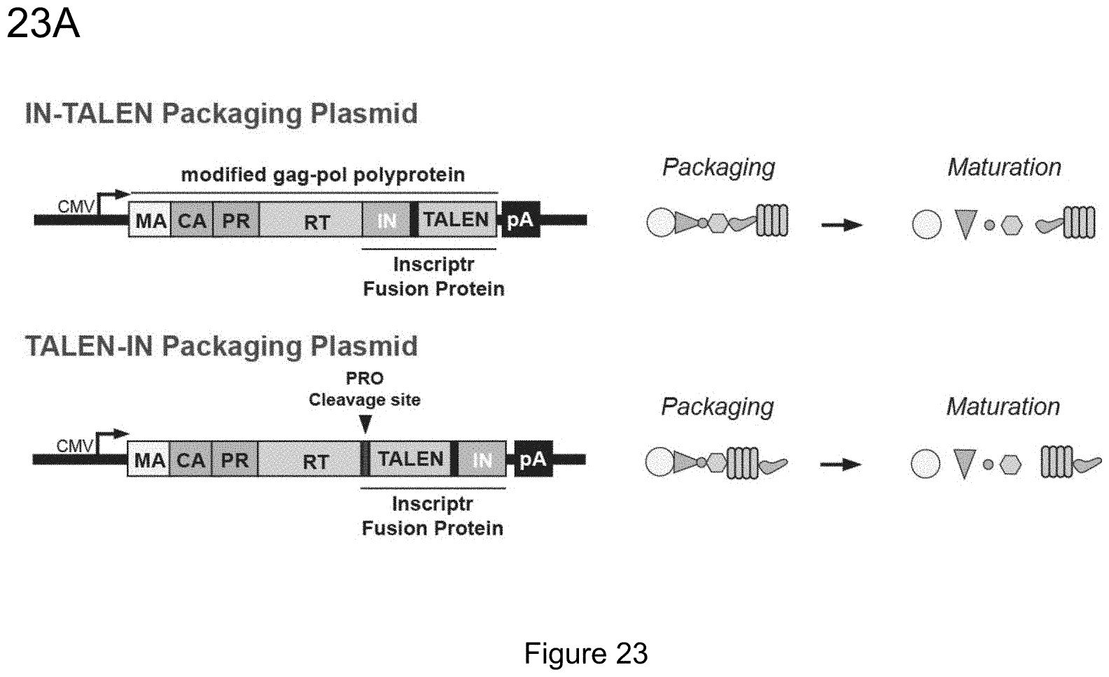

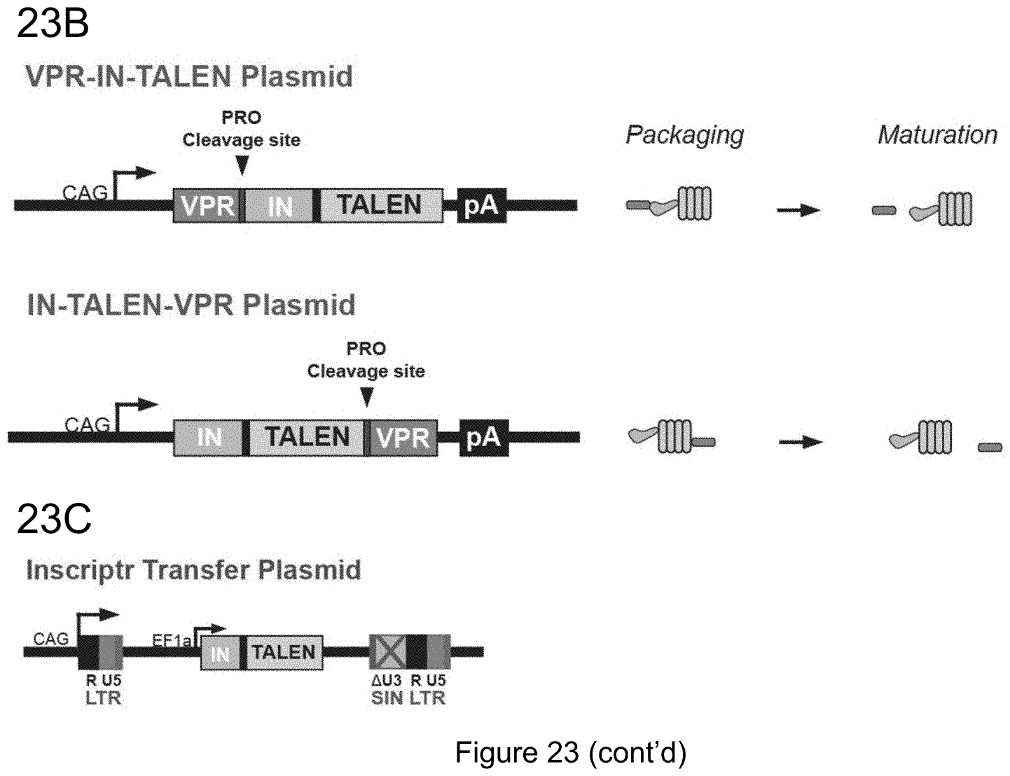

This data has demonstrated that replacement of the non-specific DNA binding domain of Integrase with the programmable DNA binding domain of dCas9, allows for targeted integration of dsDNA donor templates, or via delivery in lentiviral particles, for delivery of lentiviral encoded donor sequences. CRISPR-Cas systems are two-component, relying on both a Cas protein and small guide-RNA for targeting. In some instances, it may beneficial to utilize single-component DNA targeting proteins, such as TALENs, for delivery via lentiviral particles, as these are targeted solely by the encoded protein. Using a similar lentiviral production approach, replacement of dCas9 in previous packaging strategies with TALENs targeting a given sequence (for example, eGFP or a safe harbor locus), allows for lentiviral packaging and targeting without the requirement for delivery of guide-RNAs (FIG. 23). For example, TALENs are packed and delivered as a fusion to Integrase either in the context of the gag-pol polyprotein (FIG. 23A), the IN-TALEN as a fusion to a viral incorporated protein, such as VPR (FIG. 23B), or the IN-TALEN delivered within the transfer plasmid (FIG. 23C).

### Example 9: Enhanced CRISPR-Cas9 DNA Editing with the Ty1 NLS

CRISPR-Cas DNA cleavage systems are derived from bacteria and Cas proteins are both large and lack intrinsic mammalian nuclear localization signals (NLSs), preventing their efficient nuclear localization in mammalian cells.

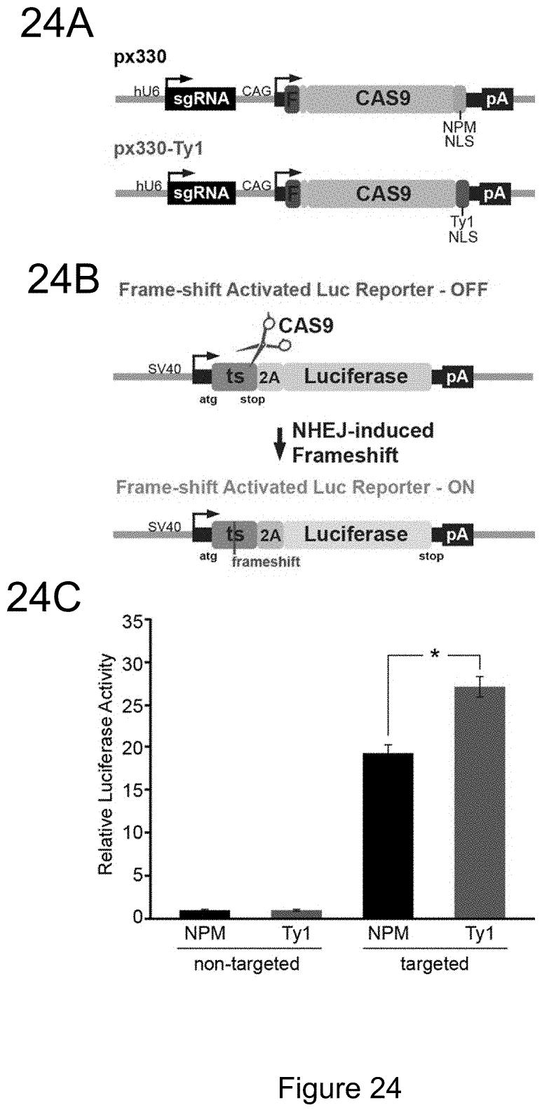

To determine if Ty1 enhances CRISPR-Cas9 editing, CRISPR-Cas9 an existing expression plasmid (px330) was modified by replacing the C-terminal NPM NLS with the non-classical Ty1 NLS (px330-Ty1) (FIG. 24A). Next a frameshift-responsive luciferase reporter was generated, which encodes an out-of-frame luciferase coding sequence downstream of a target sequence (ts)(FIG. 24B). For this reporter assay, cleavage near the target sequence and imperfect repair by the cellular non-homologous end joining (NHEJ) pathway can induce nucleotide insertions or deletions which have the potential to re-frame the luciferase coding sequence and result in luciferase expression.

Co-expression of the Luciferase reporter with a vector encoding Cas9 containing the NPM NLS and a single guide-RNA specific to a 20 nucleotide target sequence resulted in a ˜20-fold increase in luciferase activity over background, relative to a non-targeting guide-RNA (FIG. 24C). Notably, expression of Cas9 containing the Ty1 NLS resulted in a significant (˜44%) enhancement in reporter activity in COS-7 cells, compared to Cas9 containing the NPM NLS (FIG. 24C).

### Example 10: Non-Homologous DNA Integration with Integrase-TALEN Fusion Proteins

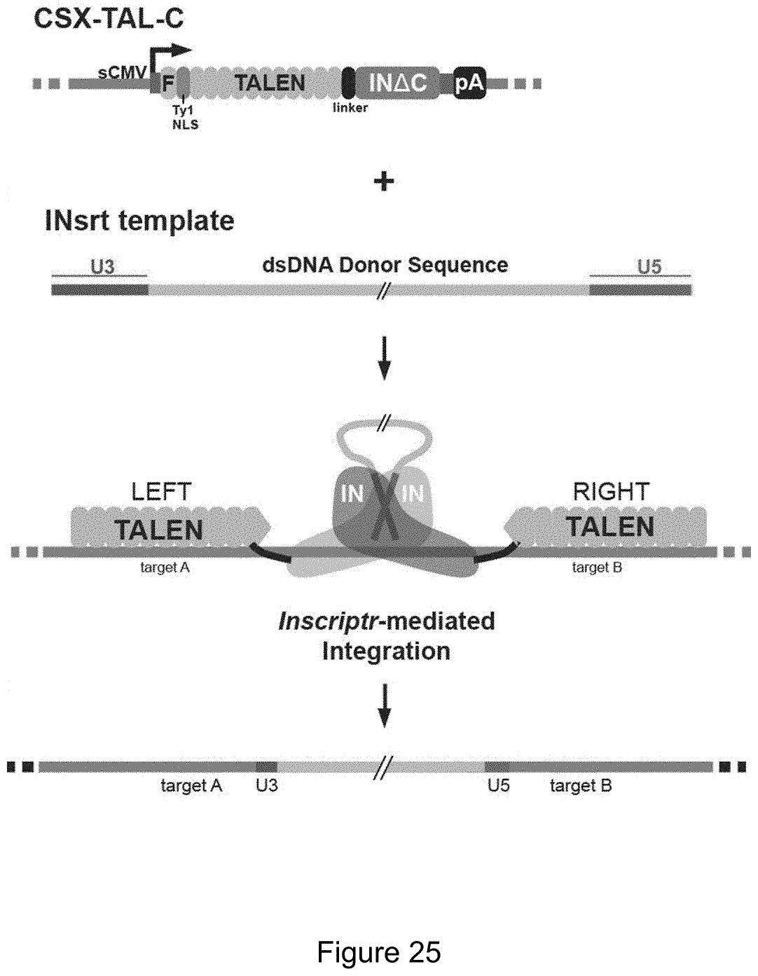

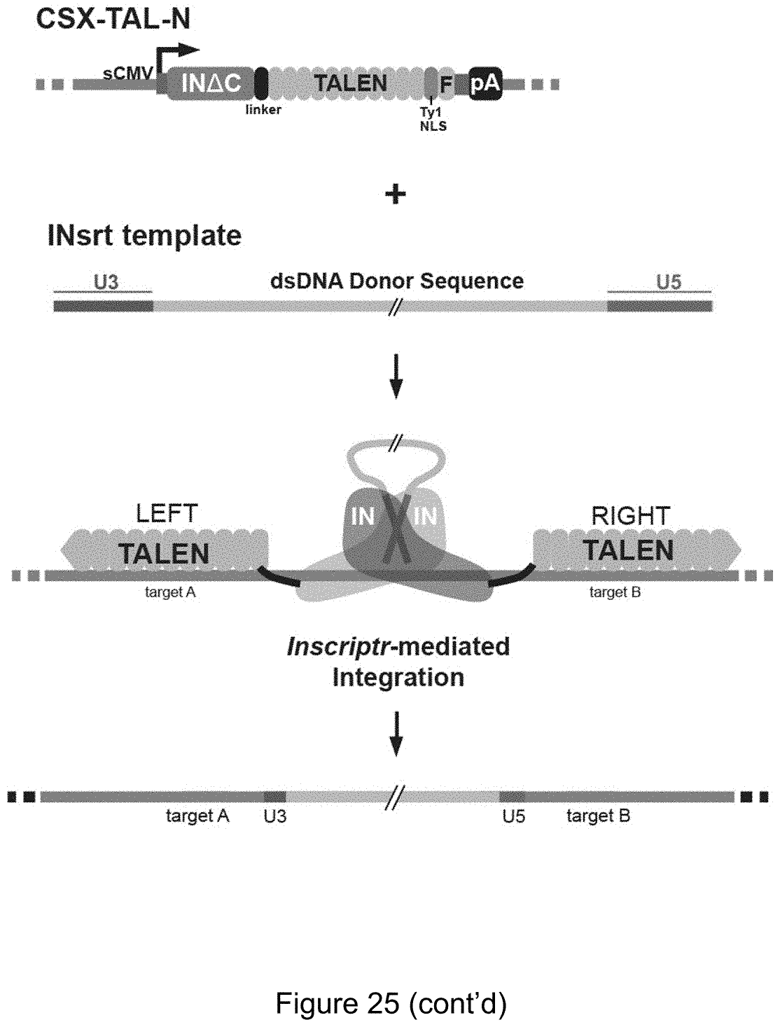

Transcription Activator-like Effector Nucleases (TALENs) are a well-studied programmable DNA binding proteins which are constructed by the tandem assembly of individual nucleotide-targeting domains (Reyon et al., 2012). In a similar approach demonstrated for Inscriptr, TALENs can be utilized to direct retroviral integrase-mediated integration of a donor DNA template (FIG. 25). To generate TALEN-Integrase fusion proteins, mammalian expression vectors were constructed to receive TALEN targeting repeats from TALEN expression vectors previously described, to generate either IN-TALEN or TALEN-IN fusions. Each fusion protein incorporated a 3×FLAG epitope, a Ty1 NLS, and a TALEN repeat separated by a linker sequence between HIV Integrase lacking the C-terminal non-specific DNA binding domain (INΔC). In some instances, IN mutations can be incorporated to alter IN activity, dimerization, interaction with cellular proteins, resistance to dimerization inhibitors or tandem copies of INΔC (tdINΔC). For example, the E85G mutation can be incorporated to inhibit obligate dimer formation.

TALEN pairs targeting eGFP have been previously described and verified for targeting efficiency (Reyon et al., 2012; available from Addgene). TALEN pairs (ClaI/BamHI fragment) were subcloned to generate TALEN-IN fusion proteins directed to eGFP with spacers either of 16 bp or 28 bp in length.

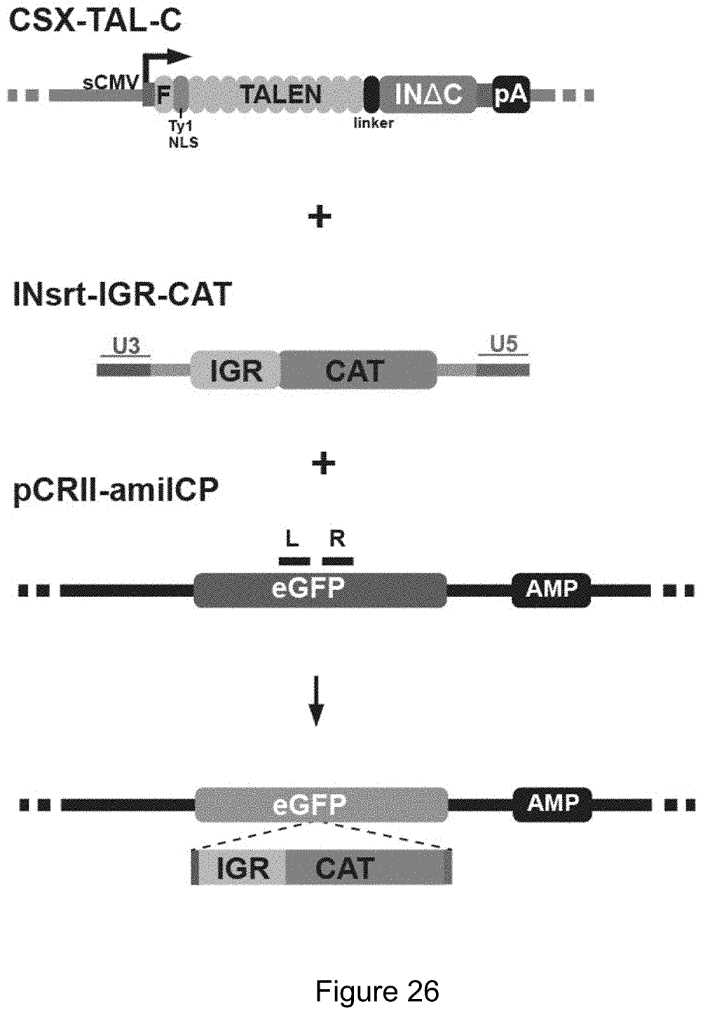

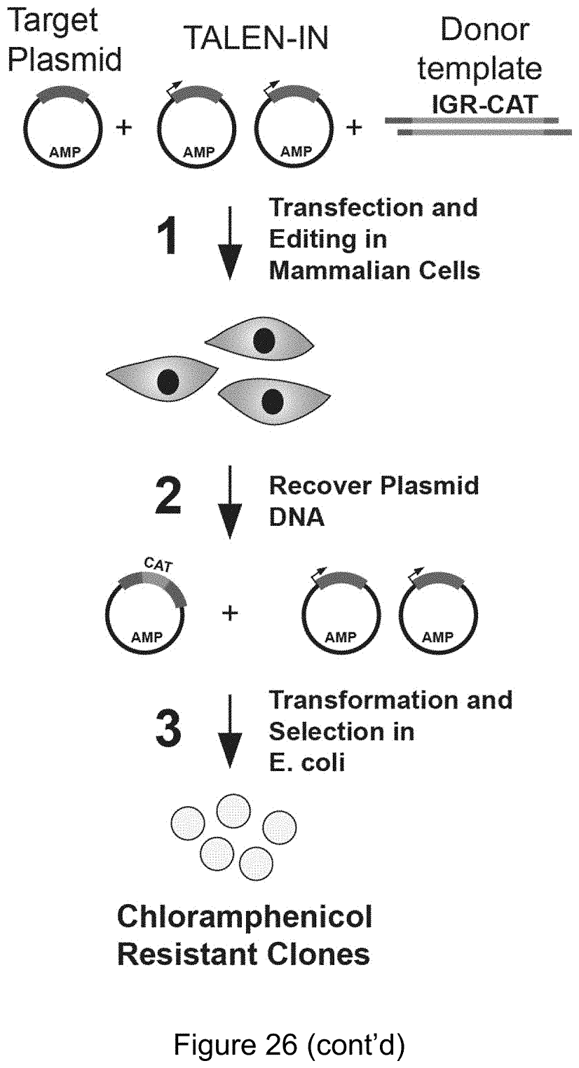

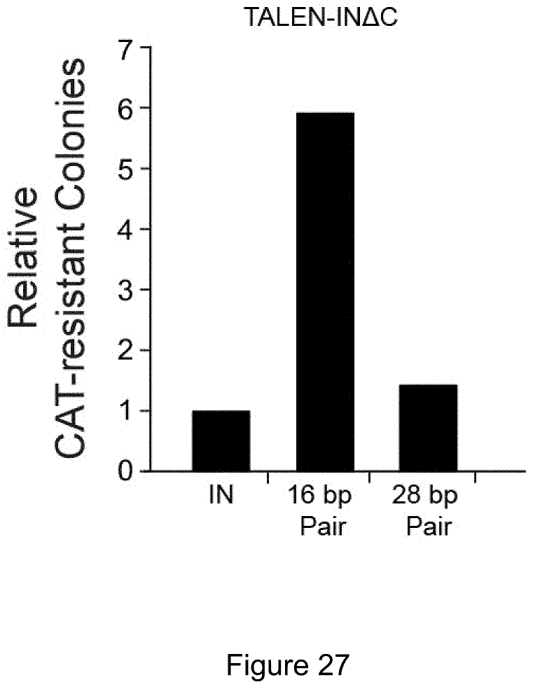

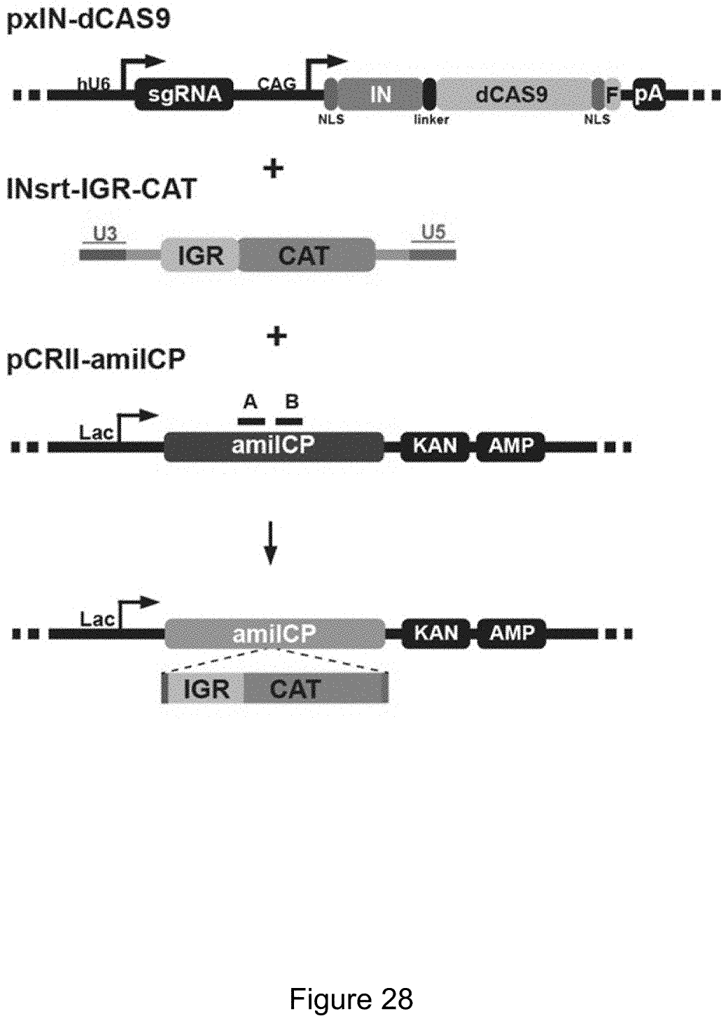

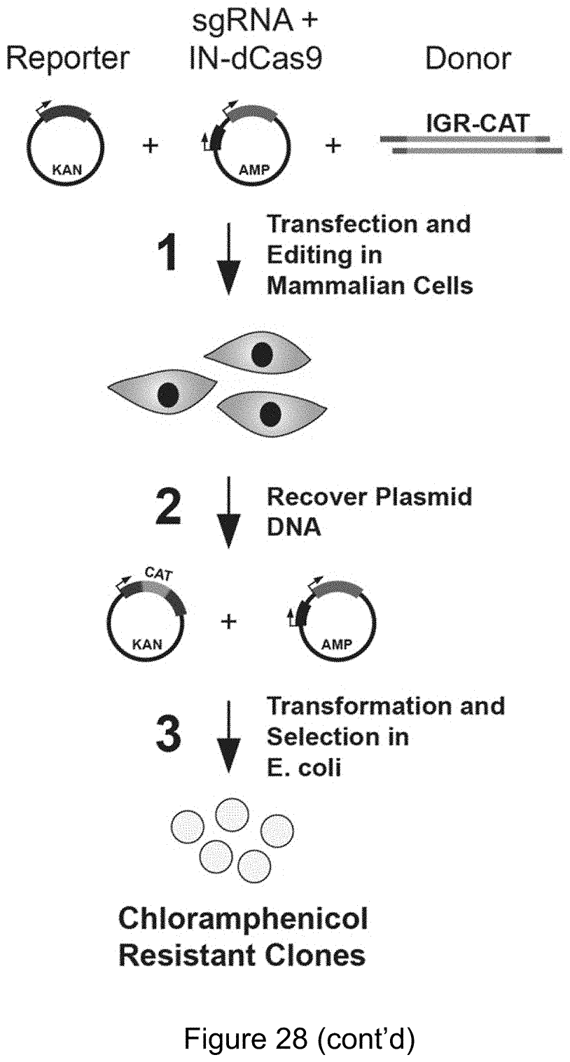

Using a plasmid DNA integration assay (FIG. 26), co-transfection of TALEN-IN pairs targeting eGFP, a linear double stranded DNA donor sequence encoding a IGR-CAT resistance gene and an amilCP bacterial expression reporter were co-transfected into mammalian COS-7 cells. Two days post-transfection, edited plasmids were recovered from mammalian cells and transformed into E. coli and selected for on chloramphenicol plates. Interestingly, a TALEN pair separated by 16 bp resulted in ˜6 fold more Chloramphenicol-resistant colonies, whereas a TALEN pair separated by 28 bp was similar to untargeted integrase (FIG. 27). These data suggest that proximity of TALEN pairs is important for targeting and integration, a feature which has been previously reported for TALEN-FokI mediated dsDNA cleavage.

### Example 11

Table of Sequences

| SEQ | | |
|---|---|---|
| ID NO | Type | Description |
| 1 | Protein | HIV IN |
| 2 | Protein | HIV INΔC |
| 3 | Protein | HIV tdINΔC |
| 4 | Protein | HIV E85G IN |
| 5 | Protein | HIV E85G INΔC |
| 6 | Protein | HIV E85F IN |
| 7 | Protein | HIV E85F INΔC |
| 8 | Protein | HIV D116N IN |
| 9 | Protein | HIV D116N INΔC |
| 10 | Protein | HIV F185K:C280S IN |
| 11 | Protein | HIV C280S IN |
| 12 | Protein | HIV F185K IN |
| 13 | Protein | HIV F185K INΔC |
| 14 | Protein | HIV T97A:Y143R IN |
| 15 | Protein | HIV T97A:Y143R INΔC |
| 16 | Protein | HIV G140S:Q148H IN |
| 17 | Protein | HIV G140S:Q148H INΔC |
| 18 | Protein | RSV IN |
| 19 | Protein | RSV INΔC |
| 20 | Protein | HFV IN |
| 21 | Protein | HFV INΔC |
| 22 | Protein | EIAV IN |
| 23 | Protein | EIAV INΔC |
| 24 | Protein | MoLV IN |
| 25 | Protein | MoLV INΔC |
| 26 | Protein | MMTV IN |
| 27 | Protein | MMTV INΔC |
| 28 | Protein | WDSV IN |
| 29 | Protein | WDSV INΔC |
| 30 | Protein | BLV IN |
| 31 | Protein | BLV INΔC |
| 32 | Protein | SIV IN |
| 33 | Protein | SIV INΔC |
| 34 | Protein | FIV IN |
| 35 | Protein | FIV INΔC |
| 36 | Protein | BIV IN |
| 37 | Protein | BIV INΔC |
| 38 | Protein | Ty1 INΔC |
| 39 | Protein | InsF IN |
| 40 | Protein | InsF INΔN |
| 41 | Protein | Cas9 |
| 42 | Protein | dCas9 |
| 43 | Protein | SaCas9 |
| 44 | Protein | dSaCas9 |
| 45 | Protein | Cpf1 |
| 46 | Protein | dCpf1 |
| 47 | Protein | 1xSV40 |
| 48 | Protein | 3xSV40 |
| 49 | Protein | 3xFLAG |
| 50 | Protein | NPM |
| 51 | Protein | Ty1 |
| 52 | Protein | 1xSV40 + 3xFLAG |
| 53 | Protein | 3xSV40 + 3xFLAG |
| 54 | Protein | NPM + 3xFLAG |
| 55 | Protein | NPM + 3xSV40 + 3xFLAG |
| 56 | Protein | Ty1 + 3xFLAG |
| 57 | Protein | HIV IN-dCas9-Ty1 |
| 58 | Protein | HIV INΔC-dCas9-Ty1 |
| 59 | Protein | HIV tdINΔC-dCas9-Ty1 |
| 60 | Protein | HIV E85G IN-dCas9-Ty1 |
| 61 | Protein | HIV E85G INΔC-dCas9-Ty1 |
| 62 | Protein | HIV E85F IN-dCas9-Ty1 |
| 63 | Protein | HIV E85F INΔC-dCas9-Ty1 |
| 64 | Protein | HIV D116N IN-dCas9-Ty1 |
| 65 | Protein | HIV D116N INΔC-dCas9-Ty1 |
| 66 | Protein | HIV F185K:C280S IN-dCas9-Ty1 |
| 67 | Protein | HIV C280S IN-dCas9-Ty1 |
| 68 | Protein | HIV F185K IN-dCas9-Ty1 |
| 69 | Protein | HIV F185K INΔC-dCas9-Ty1 |
| 70 | Protein | HIV T97A:Y143R IN-dCas9-Ty1 |
| 71 | Protein | HIV T97A:Y143R INΔC-dCas9-Ty1 |
| 72 | Protein | HIV G140S:Q148H IN-dCas9-Ty1 |
| 73 | Protein | HIV G140S:Q148H INΔC-dCas9-Ty1 |
| 74 | Protein | RSV IN-dCas9-Ty1 |
| 75 | Protein | RSV INΔC-dCas9-Ty1 |
| 76 | Protein | HFV IN-dCas9-Ty1 |
| 77 | Protein | HFV INΔC-dCas9-Ty1 |
| 78 | Protein | EIAV IN-dCas9-Ty1 |
| 79 | Protein | EIAV INΔC-dCas9-Ty1 |
| 80 | Protein | MoLV IN-dCas9-Ty1 |
| 81 | Protein | MoLV INΔC-dCas9-Ty1 |
| 82 | Protein | MMTV IN-dCas9-Ty1 |
| 83 | Protein | MMTV INΔC-dCas9-Ty1 |
| 84 | Protein | WDSV IN-dCas9-Ty1 |
| 85 | Protein | WDSV INΔC-dCas9-Ty1 |
| 86 | Protein | BLV IN-dCas9-Ty1 |
| 87 | Protein | BLV INΔC-dCas9-Ty1 |
| 88 | Protein | SIV IN-dCas9-Ty1 |
| 89 | Protein | SIV INΔC-dCas9-Ty1 |
| 90 | Protein | FIV IN-dCas9-Ty1 |
| 91 | Protein | FIV INΔC-dCas9-Ty1 |
| 92 | Protein | BIV IN-dCas9-Ty1 |
| 93 | Protein | BV INΔC-dCas9-Ty1 |
| 94 | Protein | Ty1 INΔC-dCas9-Ty1 |
| 95 | Protein | InsF IN-dCas9-Ty1 |
| 96 | Protein | InsF INΔN-dCas9-Ty1 |
| 97 | Protein | 3xFLAG-Ty1NLS-dCas9-linker-INdC |
| 98 | Protein | NLS - INdC(HIV)-linker-dSaCas9- |
| | | Ty1nls-3xFlag |
| 99 | Nucleic Acid | HIV IN |
| 100 | Nucleic Acid | HIV INΔC |
| 101 | Nucleic Acid | HIV tdINΔC |
| 102 | Nucleic Acid | HIV E85G IN |
| 103 | Nucleic Acid | HIV E85G INΔC |
| 104 | Nucleic Acid | HIV E85F IN |
| 105 | Nucleic Acid | HIV E85F INΔC |
| 106 | Nucleic Acid | HIV D116N IN |
| 107 | Nucleic Acid | HIV D116N INΔC |
| 108 | Nucleic Acid | HIV F185K:C280S IN |
| 109 | Nucleic Acid | HIV C280S IN |
| 110 | Nucleic Acid | HIV F185K IN |
| 111 | Nucleic Acid | HIV F185K INΔC |
| 112 | Nucleic Acid | HIV T97A:Y143R IN |
| 113 | Nucleic Acid | HIV T97A:Y143R INΔC |
| 114 | Nucleic Acid | HIV G140S:Q148H IN |
| 115 | Nucleic Acid | HIV G140S:Q148H INΔC |
| 116 | Nucleic Acid | RSV IN |
| 117 | Nucleic Acid | RSV INΔC |
| 118 | Nucleic Acid | HFV IN |
| 119 | Nucleic Acid | HFV INΔC |
| 120 | Nucleic Acid | EIAV IN |
| 121 | Nucleic Acid | EIAV INΔC |
| 122 | Nucleic Acid | MoLV IN |
| 123 | Nucleic Acid | MoLV INΔC |
| 124 | Nucleic Acid | MMTV IN |
| 125 | Nucleic Acid | MMTV INΔC |
| 126 | Nucleic Acid | WDSV IN |
| 127 | Nucleic Acid | WDSV INΔC |
| 128 | Nucleic Acid | BLV IN |
| 129 | Nucleic Acid | BLV INΔC |
| 130 | Nucleic Acid | SIV IN |
| 131 | Nucleic Acid | SIV INΔC |
| 132 | Nucleic Acid | FIV IN |
| 133 | Nucleic Acid | FIV INΔC |
| 134 | Nucleic Acid | BIV IN |
| 135 | Nucleic Acid | BV INΔC |
| 136 | Nucleic Acid | Ty1 INΔC |
| 137 | Nucleic Acid | InsF IN |
| 138 | Nucleic Acid | InsF INΔN |
| 139 | Nucleic Acid | Cas9 |
| 140 | Nucleic Acid | dCas9 |
| 141 | Nucleic Acid | SaCas9 |
| 142 | Nucleic Acid | dSaCas9 |
| 143 | Nucleic Acid | Cpf1 |
| 144 | Nucleic Acid | dCpf1 |
| 145 | Nucleic Acid | 1xSV40 |
| 146 | Nucleic Acid | 3xSV40 |
| 147 | Nucleic Acid | 3xFLAG |
| 148 | Nucleic Acid | NPM |
| 149 | Nucleic Acid | Ty1 |
| 150 | Nucleic Acid | 1xSV40 + 3xFLAG |
| 151 | Nucleic Acid | 3xSV40 + 3xFLAG |
| 152 | Nucleic Acid | NPM + 3xFLAG |
| 153 | Nucleic Acid | NPM + 3xSV40 + 3xFLAG |
| 154 | Nucleic Acid | Ty1 + 3xFLAG |
| 155 | Nucleic Acid | HIV IN-dCas9-Ty1 |
| 156 | Nucleic Acid | HIV INΔC-dCas9-Ty1 |
| 157 | Nucleic Acid | HIV tdINΔC-dCas9-Ty1 |
| 158 | Nucleic Acid | HIV E85G IN-dCas9-Ty1 |
| 159 | Nucleic Acid | HIV E85G INΔC-dCas9-Ty1 |
| 160 | Nucleic Acid | HIV E85F IN-dCas9-Ty1 |
| 161 | Nucleic Acid | HIV E85F INΔC-dCas9-Ty1 |
| 162 | Nucleic Acid | HIV D116N IN-dCas9-Ty1 |
| 163 | Nucleic Acid | HIV D116N INΔC-dCas9-Ty1 |
| 164 | Nucleic Acid | HIV F185K:C280S IN-dCas9-Ty1 |
| 165 | Nucleic Acid | HIV C280S IN-dCas9-Ty1 |
| 166 | Nucleic Acid | HIV F185K IN-dCas9-Ty1 |
| 167 | Nucleic Acid | HIV F185K INΔC-dCas9-Ty1 |
| 168 | Nucleic Acid | HIV T97A:Y143R IN-dCas9-Ty1 |
| 169 | Nucleic Acid | HIV T97A:Y143R INΔC-dCas9-Ty1 |
| 170 | Nucleic Acid | HIV G140S:Q148H IN-dCas9-Ty1 |
| 171 | Nucleic Acid | HIV G140S:Q148H INΔC-dCas9-Ty1 |
| 172 | Nucleic Acid | RSV IN-dCas9-Ty1 |
| 173 | Nucleic Acid | RSV INΔC-dCas9-Ty1 |
| 174 | Nucleic Acid | HFV IN-dCas9-Ty1 |
| 175 | Nucleic Acid | HFV INΔC-dCas9-Ty1 |
| 176 | Nucleic Acid | EIAV IN-dCas9-Ty1 |
| 177 | Nucleic Acid | EIAV INΔC-dCas9-Ty1 |
| 178 | Nucleic Acid | MoLV IN-dCas9-Ty1 |
| 179 | Nucleic Acid | MoLV INΔC-dCas9-Ty1 |
| 180 | Nucleic Acid | MMTV IN-dCas9-Ty1 |
| 181 | Nucleic Acid | MMTV INΔC-dCas9-Ty1 |
| 182 | Nucleic Acid | WDSV IN-dCas9-Ty1 |
| 183 | Nucleic Acid | WDSV INΔC-dCas9-Ty1 |
| 184 | Nucleic Acid | BLV IN-dCas9-Ty1 |
| 185 | Nucleic Acid | BLV INΔC-dCas9-Ty1 |
| 186 | Nucleic Acid | SIV IN-dCas9-Ty1 |
| 187 | Nucleic Acid | SIV INΔC-dCas9-Ty1 |
| 188 | Nucleic Acid | FIV IN-dCas9-Ty1 |
| 189 | Nucleic Acid | FIV INΔC-dCas9-Ty1 |
| 190 | Nucleic Acid | BIV IN-dCas9-Ty1 |
| 191 | Nucleic Acid | BV INΔC-dCas9-Ty1 |
| 192 | Nucleic Acid | Ty1 INΔC-dCas9-Ty1 |
| 193 | Nucleic Acid | InsF IN-dCas9-Ty1 |
| 194 | Nucleic Acid | InsF INΔN-dCas9-Ty1 |
| 195 | Nucleic Acid | 3xFLAG-Ty1NLS-dCas9-linker-INdC |
| 196 | Nucleic Acid | NLS - INdC(HIV)-linker-dSaCas9- |
| | | Ty1nls-3xFlag |
| 197 | Nucleic Acid | HIV U3 |
| 198 | Nucleic Acid | HIV U5 |
| 199 | Nucleic Acid | RSV U3 |
| 200 | Nucleic Acid | RSV U5 |
| 201 | Nucleic Acid | HFV U3 |
| 202 | Nucleic Acid | HFV U5 |
| 203 | Nucleic Acid | EIAV U3 |
| 204 | Nucleic Acid | EIAV U5 |
| 205 | Nucleic Acid | MoLV U3 |
| 206 | Nucleic Acid | MoLV U5 |
| 207 | Nucleic Acid | MMTV U3 |
| 208 | Nucleic Acid | MMTV U5 |
| 209 | Nucleic Acid | WDSV U3 |
| 210 | Nucleic Acid | WDSV U5 |
| 211 | Nucleic Acid | BLV U3 |
| 212 | Nucleic Acid | BLV U5 |
| 213 | Nucleic Acid | SIV U3 |
| 214 | Nucleic Acid | SIV U5 |
| 215 | Nucleic Acid | FIV U3 |
| 216 | Nucleic Acid | FIV U5 |
| 217 | Nucleic Acid | BIV U3 |
| 218 | Nucleic Acid | BIV U5 |
| 219 | Nucleic Acid | TY1 U3 |
| 220 | Nucleic Acid | TY1 U5 |
| 221 | Nucleic Acid | InsF IS3 IRL |
| 222 | Nucleic Acid | InsF IS3 IRR |
| 223 | Nucleic Acid | INsrt HIV empty vector |
| 224 | Nucleic Acid | INsrt RSV empty vector |
| 225 | Nucleic Acid | INsrt MoLV empty vector: |
| 226 | Nucleic Acid | INsrt MMTV empty vector |
| 227 | Nucleic Acid | INsrt BLV empty vector |
| 228 | Nucleic Acid | INsrt WDSV empty vector |
| 229 | Nucleic Acid | INsrt EIAV empty vector |
| 230 | Nucleic Acid | INsrt SIV empty vector |
| 231 | Nucleic Acid | INsrt FIV empty vector |
| 232 | Nucleic Acid | INsrt BIV empty vector |
| 233 | Nucleic Acid | INsrt HFV empty vector |
| 234 | Nucleic Acid | INsrt Ty1 empty vector |
| 235 | Nucleic Acid | INsrt IS3 empty vector (for InsF) |
| 236 | Nucleic Acid | INsrt(HIV)-IG3-CmR |
| 237 | Nucleic Acid | INsrt(HIV)-IG3-mCherry-2a-Puro-pA |
| 238 | Nucleic Acid | amilCP ORF target sequence |
| 239 | Nucleic Acid | amilCP open reading frame IN pCRII |
| | | backbone |
| 240 | Nucleic Acid | eGFP ORF target sequence |
| 241 | Nucleic Acid | eGFP ORF target sequence |
| 242 | Nucleic Acid | eEF1a1 3′UTR target sequence |
| 243 | Nucleic Acid | amilCP target A |
| 244 | Nucleic Acid | amilCP target B |
| 245 | Nucleic Acid | GFP target A |
| 246 | Nucleic Acid | GFP target B |
| 247 | Nucleic Acid | eEF1A1 3′UTR target A |
| 248 | Nucleic Acid | eEF1A1 3′UTR target B |
| 249 | Protein | CRISPR-Ty1 Fusion: 3XFLAG-SV40 |
| | | NLS-Cas9-NPM NLS |
| 250 | Protein | CRISPR-Ty1 Fusion: 3XFLAG-SV40 |
| | | NLS-Cas9-Ty1 NLS |
| 251 | Protein | VPR- INDC-dCas9 |
| 252 | Protein | INDC-dCas9-VPR |
| 253 | Protein | VPR |
| 254 | Protein | TY2 |
| 255 | Protein | INO4 |
| 256 | Protein | MAK11 |
| 257 | Protein | STH1 |
| 258 | Nucleic Acid | CRISPR-Ty1 Fusion: 3XFLAG-SV40 |
| | | NLS-Cas9-NPM NLS |
| 259 | Nucleic Acid | CRISPR-Ty1 Fusion: 3XFLAG-SV40 |
| | | NLS-Cas9-Ty1 NLS |
| 260 | Nucleic Acid | VPR- INDC-dCas9 |
| 261 | Nucleic Acid | INDC-dCas9-VPR |
| 262 | Nucleic Acid | VPR |
| 263 | Nucleic Acid | ts-2a-Lucifease |
| 264 | Nucleic Acid | Lenti-IRES-tdTO |
| 265 | Nucleic Acid | INDC-dCas9-psPax2 |
| 266 | Nucleic Acid | dCas9- INDC-psPax2 |
| 267 | Nucleic Acid | INDC-TALEN(GFP-L)-psPax2 |
| 268 | Nucleic Acid | INDC-TALEN(GFP-R)-psPax2 |
| 269 | Nucleic Acid | TALEN(GFP-R)- INDC-psPax2 |
| 270 | Nucleic Acid | TALEN(GFP-L)- INDC-psPax2 |
| 271 | Nucleic Acid | Guide-RNA target sequence IN-TALEN |
| | | GFP-L |
| 272 | Nucleic Acid | Guide-RNA target sequenc IN-TALEN |
| | | GFP-R |
| 273 | Nucleic Acid | Guide-RNA target sequence INdC-TALEN |
| | | GFP-L |
| 274 | Nucleic Acid | Guide-RNA target sequenc INdC-TALEN |
| | | GFP-R |
| 275 | Protein | Ty1-like NLS O28090-0 |
| 276 | Protein | Ty1-like NLS O50087-0 |
| 277 | Protein | Ty1-like NLS O58353-0 |
| 278 | Protein | Ty1-like NLS Q57602-0 |
| 279 | Protein | Ty1-like NLS Q6L1X9-0 |
| 280 | Protein | Ty1-like NLS A0K3M1-0 |
| 281 | Protein | Ty1-like NLS A0LYZ1-0 |
| 282 | Protein | Ty1-like NLS A1B022-0 |
| 283 | Protein | Ty1-like NLS A1V8A7-0 |
| 284 | Protein | Ty1-like NLS A1VIP6-0 |
| 285 | Protein | Ty1-like NLS A2RDW6-0 |
| 286 | Protein | Ty1-like NLS A2S7H2-0 |
| 287 | Protein | Ty1-like NLS A3MRV0-0 |
| 288 | Protein | Ty1-like NLS A3NEI3-0 |
| 289 | Protein | Ty1-like NLS A3P0B7-0 |
| 290 | Protein | Ty1-like NLS A4JAN6-0 |
| 291 | Protein | Ty1-like NLS A4SUV7-0 |
| 292 | Protein | Ty1-like NLS A5FP03-0 |
| 293 | Protein | Ty1-like NLS A5ILZ2-0 |
| 294 | Protein | Ty1-like NLS A6GY20-0 |
| 295 | Protein | Ty1-like NLS A6LLI5-0 |
| 296 | Protein | Ty1-like NLS A6LQX4-0 |
| 297 | Protein | Ty1-like NLS A8F6X2-0 |
| 298 | Protein | Ty1-like NLS A8G6B7-0 |
| 299 | Protein | Ty1-like NLS A9ADI9-0 |
| 300 | Protein | Ty1-like NLS A9IJ08-0 |
| 301 | Protein | Ty1-like NLS A9IXA1-0 |
| 302 | Protein | Ty1-like NLS A9NEN2-0 |
| 303 | Protein | Ty1-like NLS B0S140-0 |
| 304 | Protein | Ty1-like NLS B1JU18-0 |
| 305 | Protein | Ty1-like NLS B1LBA1-0 |
| 306 | Protein | Ty1-like NLS B1W354-0 |
| 307 | Protein | Ty1-like NLS B1XSP7-0 |
| 308 | Protein | Ty1-like NLS B1YRC6-0 |
| 309 | Protein | Ty1-like NLS B2JIH0-0 |
| 310 | Protein | Ty1-like NLS B2T755-0 |
| 311 | Protein | Ty1-like NLS B2UEM3-0 |
| 312 | Protein | Ty1-like NLS B3PLU0-0 |
| 313 | Protein | Ty1-like NLS B3R7T2-0 |
| 314 | Protein | Ty1-like NLS B4E5B6-0 |
| 315 | Protein | Ty1-like NLS B4S3C9-0 |
| 316 | Protein | Ty1-like NLS B7IHT4-0 |
| 317 | Protein | Ty1-like NLS B8E0X6-0 |
| 318 | Protein | Ty1-like NLS B9K7W0-0 |
| 319 | Protein | Ty1-like NLS C1A494-0 |
| 320 | Protein | Ty1-like NLS C5CE41-0 |
| 321 | Protein | Ty1-like NLS O88058-0 |
| 322 | Protein | Ty1-like NLS P0DG92-0 |
| 323 | Protein | Ty1-like NLS P0DG93-0 |
| 324 | Protein | Ty1-like NLS P60554-0 |
| 325 | Protein | Ty1-like NLS P67354-0 |
| 326 | Protein | Ty1-like NLS P75311-0 |
| 327 | Protein | Ty1-like NLS P75471-0 |
| 328 | Protein | Ty1-like NLS P94372-0 |
| 329 | Protein | Ty1-like NLS Q056Y0-0 |
| 330 | Protein | Ty1-like NLS Q057D7-0 |
| 331 | Protein | Ty1-like NLS Q0AYB7-0 |
| 332 | Protein | Ty1-like NLS Q0BJ50-0 |
| 333 | Protein | Ty1-like NLS Q0K610-0 |
| 334 | Protein | Ty1-like NLS Q0STA4-0 |
| 335 | Protein | Ty1-like NLS Q0STL9-0 |
| 336 | Protein | Ty1-like NLS Q0TQV7-0 |
| 337 | Protein | Ty1-like NLS Q0TR88-0 |
| 338 | Protein | Ty1-like NLS Q12GX5-0 |
| 339 | Protein | Ty1-like NLS Q13TG6-0 |
| 340 | Protein | Ty1-like NLS Q1AWG1-0 |
| 341 | Protein | Ty1-like NLS Q1BRU4-0 |
| 342 | Protein | Ty1-like NLS Q1J5X5-0 |
| 343 | Protein | Ty1-like NLS Q1JAY8-0 |
| 344 | Protein | Ty1-like NLS Q1JG57-0 |
| 345 | Protein | Ty1-like NLS Q1JL34-0 |
| 346 | Protein | Ty1-like NLS Q1LI28-0 |
| 347 | Protein | Ty1-like NLS Q2L2H3-0 |
| 348 | Protein | Ty1-like NLS Q2NIH1-0 |
| 349 | Protein | Ty1-like NLS Q2SU23-0 |
| 350 | Protein | Ty1-like NLS Q39KH1-0 |
| 351 | Protein | Ty1-like NLS Q3JMQ8-0 |
| 352 | Protein | Ty1-like NLS Q3YRL8-0 |
| 353 | Protein | Ty1-like NLS Q46WD9-0 |
| 354 | Protein | Ty1-like NLS Q48SQ4-0 |
| 355 | Protein | Ty1-like NLS Q49418-0 |
| 356 | Protein | Ty1-like NLS Q56307-0 |
| 357 | Protein | Ty1-like NLS Q5LEQ4-0 |
| 358 | Protein | Ty1-like NLS Q5WEJ7-0 |
| 359 | Protein | Ty1-like NLS Q5XBA0-0 |
| 360 | Protein | Ty1-like NLS Q62GK1-0 |
| 361 | Protein | Ty1-like NLS Q63Q07-0 |
| 362 | Protein | Ty1-like NLS Q64VP0-0 |
| 363 | Protein | Ty1-like NLS Q6G3V1-0 |
| 364 | Protein | Ty1-like NLS Q6G5M0-0 |
| 365 | Protein | Ty1-like NLS Q6LLQ8-0 |
| 366 | Protein | Ty1-like NLS Q6MDC1-0 |
| 367 | Protein | Ty1-like NLS Q6MDH4-0 |
| 368 | Protein | Ty1-like NLS Q6ME08-0 |
| 369 | Protein | Ty1-like NLS Q73PH4-0 |
| 370 | Protein | Ty1-like NLS Q7MAD1-0 |
| 371 | Protein | Ty1-like NLS Q7UP72-0 |
| 372 | Protein | Ty1-like NLS Q7VTD6-0 |
| 373 | Protein | Ty1-like NLS Q7W2F9-0 |
| 374 | Protein | Ty1-like NLS Q7WRC8-0 |
| 375 | Protein | Ty1-like NLS Q828D0-0 |
| 376 | Protein | Ty1-like NLS Q895M9-0 |
| 377 | Protein | Ty1-like NLS Q8AAP0-0 |
| 378 | Protein | Ty1-like NLS Q8D1X2-0 |
| 379 | Protein | Ty1-like NLS Q8K908-0 |
| 380 | Protein | Ty1-like NLS Q8P0C9-0 |
| 381 | Protein | Ty1-like NLS Q8XKR1-0 |
| 382 | Protein | Ty1-like NLS Q8XL46-0 |
| 383 | Protein | Ty1-like NLS Q8XV09-0 |
| 384 | Protein | Ty1-like NLS Q93Q47-0 |
| 385 | Protein | Ty1-like NLS Q9L0Q6-0 |
| 386 | Protein | Ty1-like NLS Q9L0Q6-1 |
| 387 | Protein | Ty1-like NLS Q9L0Q6-2 |
| 388 | Protein | Ty1-like NLS Q9L0Q6-3 |
| 389 | Protein | Ty1-like NLS Q9L0Q6-4 |
| 390 | Protein | Ty1-like NLS Q9L0Q6-5 |
| 391 | Protein | Ty1-like NLS Q9L0Q6-6 |
| 392 | Protein | Ty1-like NLS Q9X1S8-0 |
| 393 | Protein | Ty1-like NLS A1CNV8-0 |
| 394 | Protein | Ty1-like NLS A1D1R8-0 |
| 395 | Protein | Ty1-like NLS A1D731-0 |
| 396 | Protein | Ty1-like NLS A2QAX7-0 |
| 397 | Protein | Ty1-like NLS A3LQ55-0 |
| 398 | Protein | Ty1-like NLS A5DGY0-0 |
| 399 | Protein | Ty1-like NLS A5DKW3-0 |
| 400 | Protein | Ty1-like NLS A5DLG8-0 |
| 401 | Protein | Ty1-like NLS A5DY34-0 |
| 402 | Protein | Ty1-like NLS A6RBB0-0 |
| 403 | Protein | Ty1-like NLS A6RMZ2-0 |
| 404 | Protein | Ty1-like NLS A6ZL85-0 |
| 405 | Protein | Ty1-like NLS A6ZZJ1-0 |
| 406 | Protein | Ty1-like NLS A7E4K0-0 |
| 407 | Protein | Ty1-like NLS G0S8I1-0 |
| 408 | Protein | Ty1-like NLS O13527-0 |
| 409 | Protein | Ty1-like NLS O13535-0 |
| 410 | Protein | Ty1-like NLS O13658-0 |
| 411 | Protein | Ty1-like NLS O14064-0 |
| 412 | Protein | Ty1-like NLS O14076-0 |
| 413 | Protein | Ty1-like NLS O42668-0 |
| 414 | Protein | Ty1-like NLS O43068-0 |
| 415 | Protein | Ty1-like NLS O74777-0 |
| 416 | Protein | Ty1-like NLS O74862-0 |
| 417 | Protein | Ty1-like NLS O94383-0 |
| 418 | Protein | Ty1-like NLS O94487-0 |
| 419 | Protein | Ty1-like NLS O94585-0 |
| 420 | Protein | Ty1-like NLS O94652-0 |
| 421 | Protein | Ty1-like NLS P0C2I2-0 |
| 422 | Protein | Ty1-like NLS P0C2I3-0 |
| 423 | Protein | Ty1-like NLS P0C2I5-0 |
| 424 | Protein | Ty1-like NLS P0C2I6-0 |
| 425 | Protein | Ty1-like NLS P0C2I7-0 |
| 426 | Protein | Ty1-like NLS P0C2I9-0 |
| 427 | Protein | Ty1-like NLS P0C2J0-0 |
| 428 | Protein | Ty1-like NLS P0C2J1-0 |
| 429 | Protein | Ty1-like NLS P0C2J3-0 |
| 430 | Protein | Ty1-like NLS P0C2J5-0 |
| 431 | Protein | Ty1-like NLS P0CM98-0 |
| 432 | Protein | Ty1-like NLS P0CM99-0 |
| 433 | Protein | Ty1-like NLS P0CX63-0 |
| 434 | Protein | Ty1-like NLS P0CX64-0 |
| 435 | Protein | Ty1-like NLS P13902-0 |
| 436 | Protein | Ty1-like NLS P14746-0 |
| 437 | Protein | Ty1-like NLS P20484-0 |
| 438 | Protein | Ty1-like NLS P22936-0 |
| 439 | Protein | Ty1-like NLS P25384-0 |
| 440 | Protein | Ty1-like NLS P32597-0 |
| 441 | Protein | Ty1-like NLS P36006-0 |
| 442 | Protein | Ty1-like NLS P36080-0 |
| 443 | Protein | Ty1-like NLS P38112-0 |
| 444 | Protein | Ty1-like NLS P47098-0 |
| 445 | Protein | Ty1-like NLS P47100-0 |
| 446 | Protein | Ty1-like NLS P51599-0 |
| 447 | Protein | Ty1-like NLS P53119-0 |
| 448 | Protein | Ty1-like NLS P53123-0 |
| 449 | Protein | Ty1-like NLS P53125-0 |
| 450 | Protein | Ty1-like NLS Q01301-0 |
| 451 | Protein | Ty1-like NLS Q03434-0 |
| 452 | Protein | Ty1-like NLS Q03494-0 |
| 453 | Protein | Ty1-like NLS Q03612-0 |
| 454 | Protein | Ty1-like NLS Q03619-0 |
| 455 | Protein | Ty1-like NLS Q03707-0 |
| 456 | Protein | Ty1-like NLS Q03855-0 |
| 457 | Protein | Ty1-like NLS Q04214-0 |
| 458 | Protein | Ty1-like NLS Q04500-0 |
| 459 | Protein | Ty1-like NLS Q04670-0 |
| 460 | Protein | Ty1-like NLS Q04711-0 |
| 461 | Protein | Ty1-like NLS Q06132-0 |
| 462 | Protein | Ty1-like NLS Q07163-0 |
| 463 | Protein | Ty1-like NLS Q07509-0 |
| 464 | Protein | Ty1-like NLS Q07791-0 |
| 465 | Protein | Ty1-like NLS Q07793-0 |
| 466 | Protein | Ty1-like NLS Q09094-0 |
| 467 | Protein | Ty1-like NLS Q09180-0 |
| 468 | Protein | Ty1-like NLS Q09180-1 |
| 469 | Protein | Ty1-like NLS Q09180-2 |
| 470 | Protein | Ty1-like NLS Q09863-0 |
| 471 | Protein | Ty1-like NLS Q0U8V9-0 |
| 472 | Protein | Ty1-like NLS Q12088-0 |
| 473 | Protein | Ty1-like NLS Q12112-0 |
| 474 | Protein | Ty1-like NLS Q12113-0 |
| 475 | Protein | Ty1-like NLS Q12141-0 |
| 476 | Protein | Ty1-like NLS Q12193-0 |
| 477 | Protein | Ty1-like NLS Q12269-0 |
| 478 | Protein | Ty1-like NLS Q12273-0 |
| 479 | Protein | Ty1-like NLS Q12316-0 |
| 480 | Protein | Ty1-like NLS Q12337-0 |
| 481 | Protein | Ty1-like NLS Q12339-0 |
| 482 | Protein | Ty1-like NLS Q12414-0 |
| 483 | Protein | Ty1-like NLS Q12472-0 |
| 484 | Protein | Ty1-like NLS Q12490-0 |
| 485 | Protein | Ty1-like NLS Q12491-0 |
| 486 | Protein | Ty1-like NLS Q12501-0 |
| 487 | Protein | Ty1-like NLS Q1DNW5-0 |
| 488 | Protein | Ty1-like NLS Q1EA54-0 |
| 489 | Protein | Ty1-like NLS Q2HFA6-0 |
| 490 | Protein | Ty1-like NLS Q2HFA6-1 |
| 491 | Protein | Ty1-like NLS Q2UQI6-0 |
| 492 | Protein | Ty1-like NLS Q4HZ42-0 |
| 493 | Protein | Ty1-like NLS Q4P6I3-0 |
| 494 | Protein | Ty1-like NLS Q4WHF8-0 |
| 495 | Protein | Ty1-like NLS Q4WRV2-0 |
| 496 | Protein | Ty1-like NLS Q4WXQ7-0 |
| 497 | Protein | Ty1-like NLS Q5A2K0-0 |
| 498 | Protein | Ty1-like NLS Q5A310-0 |
| 499 | Protein | Ty1-like NLS Q5ACW8-0 |
| 500 | Protein | Ty1-like NLS Q5B6K3-0 |
| 501 | Protein | Ty1-like NLS Q6BXL7-0 |
| 502 | Protein | Ty1-like NLS Q6C1L3-0 |
| 503 | Protein | Ty1-like NLS Q6C233-0 |
| 504 | Protein | Ty1-like NLS Q6C2J1-0 |
| 505 | Protein | Ty1-like NLS Q6C7C0-0 |
| 506 | Protein | Ty1-like NLS Q6CJY0-0 |
| 507 | Protein | Ty1-like NLS Q6CJY0-1 |
| 508 | Protein | Ty1-like NLS Q6FML5-0 |
| 509 | Protein | Ty1-like NLS Q75F02-0 |
| 510 | Protein | Ty1-like NLS Q7S2A9-0 |
| 511 | Protein | Ty1-like NLS Q7S9J4-0 |
| 512 | Protein | Ty1-like NLS Q7SFJ3-0 |
| 513 | Protein | Ty1-like NLS Q875K1-0 |
| 514 | Protein | Ty1-like NLS Q8SUT1-0 |
| 515 | Protein | Ty1-like NLS Q8SVI7-0 |
| 516 | Protein | Ty1-like NLS Q8SVI7-1 |
| 517 | Protein | Ty1-like NLS Q92393-0 |
| 518 | Protein | Ty1-like NLS Q99109-0 |
| 519 | Protein | Ty1-like NLS Q99231-0 |
| 520 | Protein | Ty1-like NLS Q99337-0 |
| 521 | Protein | Ty1-like NLS Q9USK2-0 |
| 522 | Protein | Ty1-like NLS Q9UTQ5-0 |
| 523 | Protein | Ty1-like NLS A7MD48-0 |
| 524 | Protein | Ty1-like NLS O15446-0 |
| 525 | Protein | Ty1-like NLS O15446-1 |
| 526 | Protein | Ty1-like NLS O15446-2 |
| 527 | Protein | Ty1-like NLS O43148-0 |
| 528 | Protein | Ty1-like NLS O60271-0 |
| 529 | Protein | Ty1-like NLS O75128-0 |
| 530 | Protein | Ty1-like NLS O75400-0 |
| 531 | Protein | Ty1-like NLS O75691-0 |
| 532 | Protein | Ty1-like NLS O75937-0 |
| 533 | Protein | Ty1-like NLS O76021-0 |
| 534 | Protein | Ty1-like NLS O94964-0 |
| 535 | Protein | Ty1-like NLS P23497-0 |
| 536 | Protein | Ty1-like NLS P30414-0 |
| 537 | Protein | Ty1-like NLS P42081-0 |
| 538 | Protein | Ty1-like NLS P46100-0 |
| 539 | Protein | Ty1-like NLS P51608-0 |
| 540 | Protein | Ty1-like NLS P59797-0 |
| 541 | Protein | Ty1-like NLS P82979-0 |
| 542 | Protein | Ty1-like NLS Q12830-0 |
| 543 | Protein | Ty1-like NLS Q13409-0 |
| 544 | Protein | Ty1-like NLS Q13427-0 |
| 545 | Protein | Ty1-like NLS Q15361-0 |
| 546 | Protein | Ty1-like NLS Q15361-1 |
| 547 | Protein | Ty1-like NLS Q53SF7-0 |
| 548 | Protein | Ty1-like NLS Q5M9Q1-0 |
| 549 | Protein | Ty1-like NLS Q5T3I0-0 |
| 550 | Protein | Ty1-like NLS Q5T3I0-1 |
| 551 | Protein | Ty1-like NLS Q68D10-0 |
| 552 | Protein | Ty1-like NLS Q6IPR3-0 |
| 553 | Protein | Ty1-like NLS Q6PD62-0 |
| 554 | Protein | Ty1-like NLS Q6PD62-1 |
| 555 | Protein | Ty1-like NLS Q6PD62-2 |
| 556 | Protein | Ty1-like NLS Q6S8J7-0 |
| 557 | Protein | Ty1-like NLS Q6ZU65-0 |
| 558 | Protein | Ty1-like NLS Q7Z7B0-0 |
| 559 | Protein | Ty1-like NLS Q8N9E0-0 |
| 560 | Protein | Ty1-like NLS Q8NCU4-0 |
| 561 | Protein | Ty1-like NLS Q8NFU7-0 |
| 562 | Protein | Ty1-like NLS Q96DY2-0 |
| 563 | Protein | Ty1-like NLS Q96GD3-0 |
| 564 | Protein | Ty1-like NLS Q96P65-0 |
| 565 | Protein | Ty1-like NLS Q96QC0-0 |
| 566 | Protein | Ty1-like NLS Q9BQG0-0 |
| 567 | Protein | Ty1-like NLS Q9BQG0-1 |
| 568 | Protein | Ty1-like NLS Q9BRU9-0 |
| 569 | Protein | Ty1-like NLS Q9H0S4-0 |
| 570 | Protein | Ty1-like NLS Q9H6F5-0 |
| 571 | Protein | Ty1-like NLS Q9HCK1-0 |
| 572 | Protein | Ty1-like NLS Q9HCK8-0 |
| 573 | Protein | Ty1-like NLS Q9NPI1-0 |
| 574 | Protein | Ty1-like NLS Q9NSV4-0 |
| 575 | Protein | Ty1-like NLS Q9NUL3-0 |
| 576 | Protein | Ty1-like NLS Q9NWT1-0 |
| 577 | Protein | Ty1-like NLS Q9NX58-0 |
| 578 | Protein | Ty1-like NLS Q9UGU5-0 |
| 579 | Protein | Ty1-like NLS Q9UNS1-0 |
| 580 | Protein | Ty1-like NLS Q9Y2X3-0 |
| 581 | Protein | Ty1-like NLS Q9Y6X0-0 |
| 582 | Protein | Ty1-like NLS A0A1I8M2I8-0 |
| 583 | Protein | Ty1-like NLS A1XDC0-0 |
| 584 | Protein | Ty1-like NLS A7S6A5-0 |
| 585 | Protein | Ty1-like NLS A8XI07-0 |
| 586 | Protein | Ty1-like NLS A8XI07-1 |
| 587 | Protein | Ty1-like NLS C0HKU9-0 |
| 588 | Protein | Ty1-like NLS C6KTD2-0 |
| 589 | Protein | Ty1-like NLS O16140-0 |
| 590 | Protein | Ty1-like NLS O17828-0 |
| 591 | Protein | Ty1-like NLS O17966-0 |
| 592 | Protein | Ty1-like NLS O44410-0 |
| 593 | Protein | Ty1-like NLS O44410-1 |
| 594 | Protein | Ty1-like NLS O45244-0 |
| 595 | Protein | Ty1-like NLS P0DP78-0 |
| 596 | Protein | Ty1-like NLS P0DP78-1 |
| 597 | Protein | Ty1-like NLS P0DP79-0 |
| 598 | Protein | Ty1-like NLS P0DP79-1 |
| 599 | Protein | Ty1-like NLS P0DP80-0 |
| 600 | Protein | Ty1-like NLS P0DP80-1 |
| 601 | Protein | Ty1-like NLS P0DP81-0 |
| 602 | Protein | Ty1-like NLS P0DP81-1 |
| 603 | Protein | Ty1-like NLS P14196-0 |
| 604 | Protein | Ty1-like NLS P22058-0 |
| 605 | Protein | Ty1-like NLS P26023-0 |
| 606 | Protein | Ty1-like NLS P26991-0 |
| 607 | Protein | Ty1-like NLS P35978-0 |
| 608 | Protein | Ty1-like NLS P46758-0 |
| 609 | Protein | Ty1-like NLS P46758-1 |
| 610 | Protein | Ty1-like NLS P46867-0 |
| 611 | Protein | Ty1-like NLS P54644-0 |
| 612 | Protein | Ty1-like NLS P54812-0 |
| 613 | Protein | Ty1-like NLS P83212-0 |
| 614 | Protein | Ty1-like NLS Q04621-0 |
| 615 | Protein | Ty1-like NLS Q08696-0 |
| 616 | Protein | Ty1-like NLS Q08696-1 |
| 617 | Protein | Ty1-like NLS Q08696-2 |
| 618 | Protein | Ty1-like NLS Q08696-3 |
| 619 | Protein | Ty1-like NLS Q08696-4 |
| 620 | Protein | Ty1-like NLS Q08696-5 |
| 621 | Protein | Ty1-like NLS Q08696-6 |
| 622 | Protein | Ty1-like NLS Q09223-0 |
| 623 | Protein | Ty1-like NLS Q09595-0 |
| 624 | Protein | Ty1-like NLS Q1ELU8-0 |
| 625 | Protein | Ty1-like NLS Q23120-0 |
| 626 | Protein | Ty1-like NLS Q23272-0 |
| 627 | Protein | Ty1-like NLS Q24537-0 |
| 628 | Protein | Ty1-like NLS Q27450-0 |
| 629 | Protein | Ty1-like NLS Q29DY1-0 |
| 630 | Protein | Ty1-like NLS Q4N4T9-0 |
| 631 | Protein | Ty1-like NLS Q54QQ2-0 |
| 632 | Protein | Ty1-like NLS Q54QQ2-1 |
| 633 | Protein | Ty1-like NLS Q54S20-0 |
| 634 | Protein | Ty1-like NLS Q54US6-0 |
| 635 | Protein | Ty1-like NLS Q54VU4-0 |
| 636 | Protein | Ty1-like NLS Q54XP6-0 |
| 637 | Protein | Ty1-like NLS Q551H0-0 |
| 638 | Protein | Ty1-like NLS Q557G1-0 |
| 639 | Protein | Ty1-like NLS Q55CE0-0 |
| 640 | Protein | Ty1-like NLS Q61R02-0 |
| 641 | Protein | Ty1-like NLS Q75JP5-0 |
| 642 | Protein | Ty1-like NLS Q8I5P7-0 |
| 643 | Protein | Ty1-like NLS Q8I5P7-1 |
| 644 | Protein | Ty1-like NLS Q8IBP1-0 |
| 645 | Protein | Ty1-like NLS Q8ILR9-0 |
| 646 | Protein | Ty1-like NLS Q93591-0 |
| 647 | Protein | Ty1-like NLS Q95Y36-0 |
| 648 | Protein | Ty1-like NLS Q9NBL2-0 |
| 649 | Protein | Ty1-like NLS Q9NDE8-0 |
| 650 | Protein | Ty1-like NLS Q9NDE8-1 |
| 651 | Protein | Ty1-like NLS Q9NDE8-2 |
| 652 | Protein | Ty1-like NLS Q9V5P6-0 |
| 653 | Protein | Ty1-like NLS Q9VDS6-0 |
| 654 | Protein | Ty1-like NLS Q9VGW1-0 |
| 655 | Protein | Ty1-like NLS Q9VH89-0 |
| 656 | Protein | Ty1-like NLS Q9VKM6-0 |
| 657 | Protein | Ty1-like NLS Q9VNH1-0 |
| 658 | Protein | Ty1-like NLS Q9W261-0 |
| 659 | Protein | Ty1-like NLS E1B7L7-0 |
| 660 | Protein | Ty1-like NLS Q08DU1-0 |
| 661 | Protein | Ty1-like NLS Q0III3-0 |
| 662 | Protein | Ty1-like NLS Q17QH9-0 |
| 663 | Protein | Ty1-like NLS Q29S22-0 |
| 664 | Protein | Ty1-like NLS Q2KIQ2-0 |
| 665 | Protein | Ty1-like NLS Q2KJE1-0 |
| 666 | Protein | Ty1-like NLS Q2KJE1-1 |
| 667 | Protein | Ty1-like NLS Q2TBX7-0 |
| 668 | Protein | Ty1-like NLS Q4R7K1-0 |
| 669 | Protein | Ty1-like NLS Q4R8Y5-0 |
| 670 | Protein | Ty1-like NLS Q58DE2-0 |
| 671 | Protein | Ty1-like NLS Q58DU0-0 |
| 672 | Protein | Ty1-like NLS Q5E9U4-0 |
| 673 | Protein | Ty1-like NLS Q5NVM2-0 |
| 674 | Protein | Ty1-like NLS Q5R4V4-0 |
| 675 | Protein | Ty1-like NLS Q5R8B0-0 |
| 676 | Protein | Ty1-like NLS Q5RB69-0 |
| 677 | Protein | Ty1-like NLS Q5RCE6-0 |
| 678 | Protein | Ty1-like NLS Q5TM61-0 |
| 679 | Protein | Ty1-like NLS Q767K9-0 |
| 680 | Protein | Ty1-like NLS Q7YQM3-0 |
| 681 | Protein | Ty1-like NLS Q7YQM4-0 |
| 682 | Protein | Ty1-like NLS Q7YR38-0 |
| 683 | Protein | Ty1-like NLS Q95KD7-0 |
| 684 | Protein | Ty1-like NLS Q95LG8-0 |
| 685 | Protein | Ty1-like NLS Q9N1Q7-0 |
| 686 | Protein | Ty1-like NLS A2WSD3-0 |
| 687 | Protein | Ty1-like NLS A2XVF7-0 |
| 688 | Protein | Ty1-like NLS A2XVF7-1 |
| 689 | Protein | Ty1-like NLS A2XVF7-2 |
| 690 | Protein | Ty1-like NLS A2XVF7-3 |
| 691 | Protein | Ty1-like NLS A3AVH5-0 |
| 692 | Protein | Ty1-like NLS A3AVH5-1 |
| 693 | Protein | Ty1-like NLS A3AVH5-2 |
| 694 | Protein | Ty1-like NLS A3AVH5-3 |
| 695 | Protein | Ty1-like NLS A4QJZ0-0 |
| 696 | Protein | Ty1-like NLS A4QK78-0 |
| 697 | Protein | Ty1-like NLS A4QKG5-0 |
| 698 | Protein | Ty1-like NLS A4QKQ3-0 |
| 699 | Protein | Ty1-like NLS A6MN03-0 |
| 700 | Protein | Ty1-like NLS A8MS85-0 |
| 701 | Protein | Ty1-like NLS A9XMT3-0 |
| 702 | Protein | Ty1-like NLS B8YIE8-0 |
| 703 | Protein | Ty1-like NLS F4HVZ5-0 |
| 704 | Protein | Ty1-like NLS F4IQK5-0 |
| 705 | Protein | Ty1-like NLS F4IQK5-1 |
| 706 | Protein | Ty1-like NLS O22812-0 |
| 707 | Protein | Ty1-like NLS O49323-0 |
| 708 | Protein | Ty1-like NLS O64571-0 |
| 709 | Protein | Ty1-like NLS O64639-0 |
| 710 | Protein | Ty1-like NLS O64639-1 |
| 711 | Protein | Ty1-like NLS O64639-2 |
| 712 | Protein | Ty1-like NLS O65743-0 |
| 713 | Protein | Ty1-like NLS O81072-0 |
| 714 | Protein | Ty1-like NLS P09975-0 |
| 715 | Protein | Ty1-like NLS P0C262-0 |
| 716 | Protein | Ty1-like NLS P29345-0 |
| 717 | Protein | Ty1-like NLS P50888-0 |
| 718 | Protein | Ty1-like NLS P51269-0 |
| 719 | Protein | Ty1-like NLS P51430-0 |
| 720 | Protein | Ty1-like NLS Q06FP6-0 |
| 721 | Protein | Ty1-like NLS Q06FP6-1 |
| 722 | Protein | Ty1-like NLS Q06FP6-2 |
| 723 | Protein | Ty1-like NLS Q06R72-0 |
| 724 | Protein | Ty1-like NLS Q06R98-0 |
| 725 | Protein | Ty1-like NLS Q1KVQ9-0 |
| 726 | Protein | Ty1-like NLS Q1XDL7-0 |
| 727 | Protein | Ty1-like NLS Q38873-0 |
| 728 | Protein | Ty1-like NLS Q3E8X3-0 |
| 729 | Protein | Ty1-like NLS Q3ZJ77-0 |
| 730 | Protein | Ty1-like NLS Q42438-0 |
| 731 | Protein | Ty1-like NLS Q4V3E0-0 |
| 732 | Protein | Ty1-like NLS Q66GN2-0 |
| 733 | Protein | Ty1-like NLS Q6K5K2-0 |
| 734 | Protein | Ty1-like NLS Q6YS30-0 |
| 735 | Protein | Ty1-like NLS Q84WK0-0 |
| 736 | Protein | Ty1-like NLS Q84Y18-0 |
| 737 | Protein | Ty1-like NLS Q8H991-0 |
| 738 | Protein | Ty1-like NLS Q8RWY7-0 |
| 739 | Protein | Ty1-like NLS Q8RWY7-1 |
| 740 | Protein | Ty1-like NLS Q8VZ67-0 |
| 741 | Protein | Ty1-like NLS Q8VZN4-0 |
| 742 | Protein | Ty1-like NLS Q8W0K2-0 |
| 743 | Protein | Ty1-like NLS Q8W490-0 |
| 744 | Protein | Ty1-like NLS Q9CAE4-0 |
| 745 | Protein | Ty1-like NLS Q9FMZ4-0 |
| 746 | Protein | Ty1-like NLS Q9FMZ4-1 |
| 747 | Protein | Ty1-like NLS Q9FRI0-0 |
| 748 | Protein | Ty1-like NLS Q9LKI5-0 |
| 749 | Protein | Ty1-like NLS Q9LUJ5-0 |
| 750 | Protein | Ty1-like NLS Q9LUR0-0 |
| 751 | Protein | Ty1-like NLS Q9LVU8-0 |
| 752 | Protein | Ty1-like NLS Q9LVU8-1 |
| 753 | Protein | Ty1-like NLS Q9LYK7-0 |
| 754 | Protein | Ty1-like NLS Q9M020-0 |
| 755 | Protein | Ty1-like NLS Q9M1L7-0 |
| 756 | Protein | Ty1-like NLS Q9M3V8-0 |
| 757 | Protein | Ty1-like NLS Q9SRQ3-0 |
| 758 | Protein | Ty1-like NLS Q9ZPV5-0 |
| 759 | Protein | Ty1-like NLS B1AQJ2-0 |
| 760 | Protein | Ty1-like NLS D3ZUI5-0 |
| 761 | Protein | Ty1-like NLS D4A666-0 |
| 762 | Protein | Ty1-like NLS E1U8D0-0 |
| 763 | Protein | Ty1-like NLS G3V8T1-0 |
| 764 | Protein | Ty1-like NLS O35821-0 |
| 765 | Protein | Ty1-like NLS O88487-0 |
| 766 | Protein | Ty1-like NLS O88665-0 |
| 767 | Protein | Ty1-like NLS P61364-0 |
| 768 | Protein | Ty1-like NLS P61365-0 |
| 769 | Protein | Ty1-like NLS P83858-0 |
| 770 | Protein | Ty1-like NLS P83861-0 |
| 771 | Protein | Ty1-like NLS Q00566-0 |
| 772 | Protein | Ty1-like NLS Q05CL8-0 |
| 773 | Protein | Ty1-like NLS Q09XV5-0 |
| 774 | Protein | Ty1-like NLS Q3TFK5-0 |
| 775 | Protein | Ty1-like NLS Q3TFK5-1 |
| 776 | Protein | Ty1-like NLS Q3TFK5-2 |
| 777 | Protein | Ty1-like NLS Q3TYA6-0 |
| 778 | Protein | Ty1-like NLS Q3UMF0-0 |
| 779 | Protein | Ty1-like NLS Q498U4-0 |
| 780 | Protein | Ty1-like NLS Q4V7C4-0 |
| 781 | Protein | Ty1-like NLS Q4V8G7-0 |
| 782 | Protein | Ty1-like NLS Q505I5-0 |
| 783 | Protein | Ty1-like NLS Q562C7-0 |
| 784 | Protein | Ty1-like NLS Q566R3-0 |
| 785 | Protein | Ty1-like NLS Q566R3-1 |
| 786 | Protein | Ty1-like NLS Q566R3-2 |
| 787 | Protein | Ty1-like NLS Q58A65-0 |
| 788 | Protein | Ty1-like NLS Q5NBX1-0 |
| 789 | Protein | Ty1-like NLS Q5XG71-0 |
| 790 | Protein | Ty1-like NLS Q5XI01-0 |
| 791 | Protein | Ty1-like NLS Q5XIB5-0 |
| 792 | Protein | Ty1-like NLS Q5XIR6-0 |
| 793 | Protein | Ty1-like NLS Q60848-0 |
| 794 | Protein | Ty1-like NLS Q62018-0 |
| 795 | Protein | Ty1-like NLS Q62018-1 |
| 796 | Protein | Ty1-like NLS Q62187-0 |
| 797 | Protein | Ty1-like NLS Q62871-0 |
| 798 | Protein | Ty1-like NLS Q63520-0 |
| 799 | Protein | Ty1-like NLS Q642C0-0 |
| 800 | Protein | Ty1-like NLS Q68SB1-0 |
| 801 | Protein | Ty1-like NLS Q6AYK5-0 |
| 802 | Protein | Ty1-like NLS Q6NZB0-0 |
| 803 | Protein | Ty1-like NLS Q76KJ5-0 |
| 804 | Protein | Ty1-like NLS Q76KJ5-1 |
| 805 | Protein | Ty1-like NLS Q76KJ5-2 |
| 806 | Protein | Ty1-like NLS Q78WZ7-0 |
| 807 | Protein | Ty1-like NLS Q78WZ7-1 |
| 808 | Protein | Ty1-like NLS Q7TNB4-0 |
| 809 | Protein | Ty1-like NLS Q7TPV4-0 |
| 810 | Protein | Ty1-like NLS Q80WC1-0 |
| 811 | Protein | Ty1-like NLS Q80Z37-0 |
| 812 | Protein | Ty1-like NLS Q811R2-0 |
| 813 | Protein | Ty1-like NLS Q8BKA3-0 |
| 814 | Protein | Ty1-like NLS Q8CJ67-0 |
| 815 | Protein | Ty1-like NLS Q8K214-0 |
| 816 | Protein | Ty1-like NLS Q8K4T4-0 |
| 817 | Protein | Ty1-like NLS Q8R5F3-0 |
| 818 | Protein | Ty1-like NLS Q91X13-0 |
| 819 | Protein | Ty1-like NLS Q9CS72-0 |
| 820 | Protein | Ty1-like NLS Q9CVI2-0 |
| 821 | Protein | Ty1-like NLS Q9CWX9-0 |
| 822 | Protein | Ty1-like NLS Q9CZX5-0 |
| 823 | Protein | Ty1-like NLS Q9D1J3-0 |
| 824 | Protein | Ty1-like NLS Q9D3V1-0 |
| 825 | Protein | Ty1-like NLS Q9DBQ9-0 |
| 826 | Protein | Ty1-like NLS Q9JIX5-0 |
| 827 | Protein | Ty1-like NLS Q9JJ80-0 |
| 828 | Protein | Ty1-like NLS Q9JJ89-0 |
| 829 | Protein | Ty1-like NLS Q9R1C7-0 |
| 830 | Protein | Ty1-like NLS Q9R1X4-0 |
| 831 | Protein | Ty1-like NLS Q9Z180-0 |
| 832 | Protein | Ty1-like NLS Q9Z207-0 |
| 833 | Protein | Ty1-like NLS Q9Z2D6-0 |
| 834 | Protein | Ty1-like NLS A0A1L8GSA2-0 |
| 835 | Protein | Ty1-like NLS A0JP82-0 |
| 836 | Protein | Ty1-like NLS A1A5I1-0 |
| 837 | Protein | Ty1-like NLS A1L2T6-0 |
| 838 | Protein | Ty1-like NLS A2RUV0-0 |
| 839 | Protein | Ty1-like NLS A9JRD8-0 |
| 840 | Protein | Ty1-like NLS E7F568-0 |
| 841 | Protein | Ty1-like NLS F1QFU0-0 |
| 842 | Protein | Ty1-like NLS F1QWK4-0 |
| 843 | Protein | Ty1-like NLS K9JHZ4-0 |
| 844 | Protein | Ty1-like NLS P07193-0 |
| 845 | Protein | Ty1-like NLS P0CB65-0 |
| 846 | Protein | Ty1-like NLS P12957-0 |
| 847 | Protein | Ty1-like NLS P13505-0 |
| 848 | Protein | Ty1-like NLS P21783-0 |
| 849 | Protein | Ty1-like NLS Q28BS0-0 |
| 850 | Protein | Ty1-like NLS Q28BS0-1 |
| 851 | Protein | Ty1-like NLS Q28G05-0 |
| 852 | Protein | Ty1-like NLS Q32N87-0 |
| 853 | Protein | Ty1-like NLS Q3KPW4-0 |
| 854 | Protein | Ty1-like NLS Q4QR29-0 |
| 855 | Protein | Ty1-like NLS Q4QR29-1 |
| 856 | Protein | Ty1-like NLS Q5BL56-0 |
| 857 | Protein | Ty1-like NLS Q5XJK9-0 |
| 858 | Protein | Ty1-like NLS Q5ZIJ0-0 |
| 859 | Protein | Ty1-like NLS Q640I9-0 |
| 860 | Protein | Ty1-like NLS Q6DEU9-0 |
| 861 | Protein | Ty1-like NLS Q6DEU9-1 |
| 862 | Protein | Ty1-like NLS Q6DEU9-2 |
| 863 | Protein | Ty1-like NLS Q6DK85-0 |
| 864 | Protein | Ty1-like NLS Q6DRI7-0 |
| 865 | Protein | Ty1-like NLS Q6DRL5-0 |
| 866 | Protein | Ty1-like NLS Q6NV26-0 |
| 867 | Protein | Ty1-like NLS Q6NWI1-0 |
| 868 | Protein | Ty1-like NLS Q6NYJ3-0 |
| 869 | Protein | Ty1-like NLS Q6P4K1-0 |
| 870 | Protein | Ty1-like NLS Q6WKW9-0 |
| 871 | Protein | Ty1-like NLS Q7ZUF2-0 |
| 872 | Protein | Ty1-like NLS Q7ZW47-0 |
| 873 | Protein | Ty1-like NLS Q7ZXZ0-0 |
| 874 | Protein | Ty1-like NLS Q7ZXZ0-1 |
| 875 | Protein | Ty1-like NLS Q7ZYR8-0 |
| 876 | Protein | Ty1-like NLS Q8AVQ6-0 |
| 877 | Protein | Ty1-like NLS Q9DE07-0 |
| 878 | Protein | Ty1-like NLS P03086-0 |
| 879 | Protein | Ty1-like NLS P09814-0 |
| 880 | Protein | Ty1-like NLS P0CK10-0 |
| 881 | Protein | Ty1-like NLS P15075-0 |
| 882 | Protein | Ty1-like NLS P51724-0 |
| 883 | Protein | Ty1-like NLS P52344-0 |
| 884 | Protein | Ty1-like NLS P52531-0 |
| 885 | Protein | Ty1-like NLS Q5UP41-0 |
| 886 | Protein | Ty1-like NLS Q9DUC0-0 |
| 887 | Protein | Ty1-like NLS Q9XJS3-0 |
| 888 | Nucleic Acid | 3xFLAG-Ty1 NLS-TALEN-INDC -40L |
| 889 | Nucleic Acid | 3xFLAG-Ty1 NLS-TALEN-INDC -40R |
| 890 | Nucleic Acid | 3xFLAG-Ty1 NLS-TALEN-INDC -44R |
| 891 | Nucleic Acid | INDC-TALEN-Ty1 NLS-3xFLAG -41R |
| 892 | Nucleic Acid | INDC-TALEN-Ty1 NLS-3xFLAG-45L |
| 893 | Nucleic Acid | INDC-TALEN-Ty1 NLS-3xFLAG-45R |
| 894 | Nucleic Acid | INDC-TALEN-Ty1 NLS-3xFLAG-48L |
| 895 | Nucleic Acid | pCRII-amilCP |

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A fusion protein comprising:
a) a retroviral integrase (IN), or a fragment thereof having a first amino acid sequence;
b) a CRISPR-associated (Cas) protein having a second amino acid sequence; and
c) a nuclear localization signal (NLS) having a third amino acid sequence.

2. The fusion protein of claim 1, wherein the retroviral IN is selected from the group consisting of human immunodeficiency virus (HIV) IN, Rous sarcoma virus (RSV) IN, Mouse mammary tumor virus (MMTV) IN, Moloney murine leukemia virus (MoLV) IN, bovine leukemia virus (BLV) IN, Human T-lymphotropic virus (HTLV) IN, avian sarcoma leukosis virus (ASLV) IN, feline leukemia virus (FLV) IN, xenotropic murine leukemia virus-related virus (XMLV) IN, simian immunodeficiency virus (SIV) IN, feline immunodeficiency virus (FIV) IN, equine infectious anemia virus (EIAV) IN, Prototype foamy virus (PFV) IN, simian foamy virus (SFV) IN, human foamy virus (HFV) IN, walleye dermal sarcoma virus (WDSV) IN, and bovine immunodeficiency virus (BIV) IN.

3. The fusion protein of claim 1, wherein the retroviral IN fragment comprises the IN N-terminal domain (NTD), and the IN catalytic core domain (CCD).

4. The fusion protein of claim 1, wherein the Cas protein is selected from the group consisting of Cas9, Cas13, and Cpf1.

5. The fusion protein of claim 1, wherein the Cas protein is catalytically deficient (dCas).

6. The fusion protein of claim 1, wherein the NLS is a retrotransposon NLS.

7. The fusion protein of claim 6, wherein the retrotransposon NLS is Ty1 NLS.

8. The fusion protein of claim 1, wherein the retroviral IN comprises a sequence at least 70% identical to one of SEQ ID NOs:1-40.

9. (canceled)

10. The fusion protein of claim 1, wherein the Cas protein comprises a sequence at least 95% identical to one of SEQ ID NOs:41-46.

11. (canceled)

12. The fusion protein of claim 1, wherein the NLS comprises a sequence at least 70% identical to one of SEQ ID NOs:47-56.

13. The fusion protein of claim 1, wherein the NLS comprises a sequence selected from SEQ ID NOs:47-56.

14. The fusion protein of claim 1, wherein the fusion protein comprises a sequence at least 70% identical to one of SEQ ID NOs:57-98.

15. (canceled)

16. A nucleic acid molecule encoding a fusion protein of claim 1.

17. The nucleic acid molecule of claim 16, wherein the nucleic acid comprises a sequence at least 70% identical to one of SEQ ID NOs:155-196.

18. (canceled)

19. A method of editing genetic material, the method comprising administering to the genetic material:
a) the fusion protein of claim 1 or a nucleic acid molecule encoding the fusion protein of claim 1;
b) a guide nucleic acid comprising a targeting nucleotide sequence complimentary to a target region in the genetic material; and
c) a donor template nucleic acid comprising a U3 sequence, a U5 sequence and a donor template sequence.

20. The method of claim 19 being either an in vitro or in vivo method.

21. A system for editing genetic material, comprising in one or more vectors:
a) a nucleic acid sequence encoding a fusion protein, wherein the fusion protein comprises a retroviral integrase (IN), or a fragment thereof; a CRISPR-associated (Cas) protein, and a nuclear localization signal (NLS);
b) a nucleic acid sequence coding a CRISPR-Cas system guide RNA; and
c) a nucleic acid sequence coding a donor template nucleic acid, wherein the donor template nucleic acid comprises a U3 sequence, a U5 sequence and a donor template sequence.

22. The system of claim 21, wherein the nucleic acids of a), b) and c) are on the same or different vectors.

23. The system of claim 21, wherein the fusion protein comprises a sequence at least 95% identical to one of SEQ ID NOs:57-98.

24. (canceled)

25. The system of claim 21, wherein the CRISPR-Cas system guide RNA substantially hybridizes to a target DNA sequence in the gene.

26. The system of claim 21, wherein the U3 sequence and U5 sequence are specific to the retroviral IN.

27. A system for delivering genome editing components, the system comprising a packaging plasmid, a transfer plasmid, and an envelope plasmid, wherein:
a) the packaging plasmid comprises a sequence encoding a gag-pol polyprotein comprising integrase fused to a catalytically dead Cas (dCas) protein;
the transfer plasmid comprises a sequence encoding a donor sequence, a 5′LTR and a 3′LTR; and
the envelope plasmid comprises a nucleic acid sequence encoding an envelope protein;
b) the packaging plasmid comprises a nucleic acid sequence encoding a gag-pol polyprotein; the transfer plasmid comprises a nucleic acid sequence encoding an guide RNA, a fusion protein comprising integrase and a catalytically dead Cas, a 5′LTR and a 3′LTR; and the envelope plasmid comprises a nucleic acid sequence encoding an envelope protein; or
c) the packaging plasmid comprises a sequence encoding a gag-pol polyprotein; the transfer plasmid comprises a sequence encoding a donor sequence, a 5′LTR and a 3′LTR; the envelope plasmid comprises a nucleic acid sequence encoding an envelope protein; and the system further comprise a VPR-IN-dCas plasmid comprising a nucleic acid sequence encoding a fusion protein comprising VPR, integrase, and catalytically dead Cas (dCas).

28. The system of claim 27, wherein the system comprises the packaging plasmid comprising a sequence encoding a gag-pol polyprotein comprising integrase fused to a catalytically dead Cas (dCas) protein the transfer plasmid comprising a sequence encoding a donor sequence, a 5′LTR and a 3′LTR; and the envelope plasmid comprising a nucleic acid sequence encoding an envelope protein; and
the packaging plasmid further comprises a sequence encoding a guide RNA sequence.

29. (canceled)

30. The system of claim 27, wherein the system comprises the packaging plasmid comprising a sequence encoding a gag-pol polyprotein; the transfer plasmid comprising a sequence encoding a donor sequence, a 5′LTR and a 3′LTR; the envelope plasmid comprising a nucleic acid sequence encoding an envelope protein; and the VPR-IN-dCas plasmid comprising a nucleic acid sequence encoding a fusion protein comprising VPR, integrase, and catalytically dead Cas (dCas), wherein the VPR-IN-dCas plasmid further comprises a sequence encoding a guide RNA sequence.

31. (canceled)
